SEQUENCE LISTING (127 sequences)

SEQ ID NO: 1 - RNA, 2097 nt, Homo sapiens
  source 1..2097: mol_type = genomic RNA
gctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaa

SEQ ID NO: 2 - RNA, 5620 nt, Homo sapiens
  source 1..5620: mol_type = genomic RNA
gcagtttcagaacccagccagcctctctcttgctgcctagcctcctgccggcctcatcttcgcccagccaaccccgcctggagccctatggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgagccagtcgctgtggttgttttagctccttgactccttgtggtttatgtcatcatacatgactcagcatacctgctggtgcagagctgaagattttggagggtcctccacaataaggtcaatgccagagacggaagcctttttccccaaagtcttaaaataacttatatcatcagcatacctttattgtgatctatcaatagtcaagaaaaattattgtataagattagaatgaaaattgtatgttaagttacttcactttaattctcatgtgatccttttatgttatttatatattggtaacatcctttctattgaaaaatcaccacaccaaacctctcttattagaacaggcaagtgaagaaaagtgaatgctcaagtttttcagaaagcattacatttccaaatgaatgaccttgttgcatgatgtatttttgtacccttcctacagatagtcaaaccataaacttcatggtcatgggtcatgttggtgaaaattattctgtaggatataagctacccacgtacttggtgctttaccccaacccttccaacagtgctgtgaggttggtattatttcattttttagatgagaaaatgggagctcagagaggttatatatttaagttggtgcaaaagtaattgcaagttttgccaccgaaaggaatggcaaaaccacaattatttttgaaccaacctaataatttaccgtaagtcctacatttagtatcaagctagagactgaatttgaactcaactctgtccaactccaaaattcatgtgctttttccttctaggcctttcataccaaactaatagtagtttatattctcttccaacaaatgcatattggattaaattgactagaatggaatctggaatatagttcttctggatggctccaaaacacatgtttttcttcccccgtcttcctcctcctcttcatgctcagtgttttatatatgtagtatacagttaaaatatacttgttgctggtactggcagcttatattttctctcttttttcatggattaaccttgcttgagggctttaacaattgtattactttttcaaagaactaagctttagcttcattgatttttttctatttaattgggttttgctcttctctttagcattggaaacatagaaatgctttctgatttctttgggtagatttacgtattcagcttcttgagatggaagtttagatcactgatccttcagcttgttttcttttttgtatacatagattttaggacgatatattttcccttgagttctgctttagctgcagctcttatgttttgatatgcctctctttattatccttcagttaaaaatatctttcaattcattgttatataaaaatatgtgcctagtttttaacatctggagattttctagttttgaaaaaaacataagccaggcatggtggctcacacctgtatccccagcactttgggaggccgagacgggaggatcgcctgagctcaggagtttttacaccagcctgggaataacagtgagacattatctccaaaaaaattacctgggtatggtgttgtgcacctgtagtcccagctactctggagactgaggtgggaggattgtttgagcttgggaggttgaggctgcagggagctgtgatcacaccactgcactctggcctgagtgacagattgagaccctgtctcaataaaagcaaaaataaagaaaataaaccatatgtgttgaacaaaggattaataaattaatttgagactccttcagggaatgaccacaatttattgaaaatagcctaaatgttggagtcaggcatttctggattcatattttgacatcatgctgtcatcttgaacaaaatgcctaacctttctgaacttcaacttccttgccactcaaataaggattacaaaacttaaaatgtggtaagtactaaagacgacagcaaaaattgagtccagcacagagcttcctaaataagcaagcactcaacagagttggttcctttcttcctcccctgcttgacaatccagtttcccacaggagcctttgtagctgtagccaccatggtcagtccagggattcttcactagccccttctcccctggcagacatccttgtgggagtttagtcttggctcgacatgaggatgggggtttgggaccagttctgagtgagaatcagacttgccccaagttgccattagctccccctgcagaatgtcttcagaatcggggcccggtcagtctcctgggtgacctgctgttttcctcttaagatcctttccactttggttgctgctttcgggactcatcgagtccttgctcaacaggataccccttgaagtggctgcctgggccacatccccttccaaacaagaaatcaaaatattagaaatcaatttttgaaatttcccctaggaagactcatttgagtgttcaagttcagagccagtggagaccttaggggagggtggtcacaaggattttgcacagtgctttagagggtcccagggagccacagaggtggtgaggggctgggtgctcttttctccgtgcatgaccttgtgtgtctatcttcattaccacaatgcctcatctctacctcctttccccctgtagttccaacgtgggtatctttgccatctctggcccgaaggactttctgacctacatgtataaataccccctcacaatatatattacttttcctataagtgacttctctactggattactggttgctcatacacctcatattttactcgtaaatctactactccctgtctgcctactccattctcatttgctgtagaaaattctcttaccatcccaactttcacccaccatcatgcttacccaaaggctgtgggaatgacctgggccctaatgccccttttctaaattcctaaggctcaccattttcctattgtaatggttcttgaccttataatgtttgaggcaccttttcaaatatagtcctttgatttcagactgaatacttgaaaggacacacacacacatacgtaagtgcatatgactgcatacacccacacacacacacgtgcctgtatacagtcatatgatacatacacaaacacacgcacacaagcctgcatacatcatatgccaacagtggggatatgttctgagaaatgcatcattagatgattttgtcattgtgtgaacatcatagagtgtacttacactaacctagatggtctaacctactacacacccaggctacatggtatcacctattcctcctaggctacaagcctgtacagcgtgtgtctgtactaaatgctgtgggcaattttaacctgatggtaaatgtttgtgtatctaaacatatctaaacatagaaaaggtacagtaaacatgcagtattataatcttatgagaccgtcatcatatatgtggtccactgtttgggccatcattggctgaaaagtggttatgcgacacatgactgtatatatactttcctgttacaacaacagtgtctctcaatccacagtaattgcagcatccagtaggtcttactttagccctgagtcaccatttgtgtcaacgtgtttagtgccatgtccacgtctctcatgtaactggcagagctatcaaatattttggcaaaacacattgtttctttggctttgccttggtaactttctgtgccttttgtagctcttgtttggaagaagctcaacccatgtctgcacactgtgatacaagggggacagcatcgacatcgacttacttcttggtgccttattcctccttagaacaattcctaaatctgtaacttaagtttctcaggaagattccatactgcacagaaaactgcttttgtgggtttttaaaaggcaagttgttatatgtgctggatagtttttaagtatgacataaaaattgtataaagtaaaatattaaaatacacctagaatactgtataactttaagtcattttatcaacacattgctaatccagatattttcccgcagtttttctttgaataacagagcaattaatttacttttactatgaagagtcatcattttagtatgtattttaagcaatccaccaagaactcagtaggcagctgagaggtgctgcccagagaagtggtgattagcttggccttagctcacccacacaaagcacaacaggctttgaactattccctaacggggcatttattcttttttttttttttttttgggagacggagtctcgctgtcgcccaggctagagtgcagtggcgcgatctcggctcactgcaggctccaccccctggggttcacgccattctcctgcctcagcctcccaagtagctgggactgcaggcgcccgccatctcgcccggctaattttttgtatttttagtagagacggggtttcaccgtgttagccaggatagggcatttattcttgaacttgattcagagaggcacacattaccattctctaatcagaatgcaagtagcgcaaggcggtggaaactatggaattcggaggcaggtgatgcattgggcgagtttattaacatctgtgactctctagtttgaaatttatttgtaacagacaaaaatgaattaaacaaacaataaaagtataataaagaa

SEQ ID NO: 3 - RNA, 4557 nt, Homo sapiens
  source 1..4557: mol_type = genomic RNA
cccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaaca

SEQ ID NO: 4 - RNA, 3634 nt, Homo sapiens
  source 1..3634: mol_type = genomic RNA
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 5 - RNA, 868 nt, Homo sapiens
  source 1..868: mol_type = genomic RNA
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccaggtaatattctgaatgtgtccattaaaatatgtctaacactgtcccctagcacctagcatgatgtctgcctatcatagtcattcagtgattgttgaataaatgaatgaatgaataaca

SEQ ID NO: 6 - RNA, 3624 nt, Homo sapiens
  source 1..3624: mol_type = genomic RNA
acttcccgcttgggcgcccggacggcgaatggagcaggggcgcgcagataattaaagatttacacacagctggaagaaatcatagagaagccgggcgtggtggctcatgcctataatcccagcacttttggaggctgaggcgggcagatcacttgagatcaggagttcgagaccagcctggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctcttta

SEQ ID NO: 7 - RNA, 3735 nt, Homo sapiens
  source 1..3735: mol_type = genomic RNA
acttcccgcttgggcgcccggacggcgaatggagcaggggcgcgcaggtgccttggcatctcccaatggggtggctttgctctgggctcctgttccctgtgagctgcctggtcctgctgcaggtggcaagctctggactcttcaggatgccgtgtggagaaaggaagagggtggaagccaggaggtctggagggaggtctggagtggaggagatgagaggctccggatccctctgggaggtagatttgaggacagattggaattgaggtgaaagacagagaaagagaagtggccaggatgactccaagatttctgacctaaactactgggaaggacgcggttgtcatttctgaaatgcagaaggatgccagaagagaagggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctcttta

SEQ ID NO: 8 - RNA, 5764 nt, Homo sapiens
  source 1..5764: mol_type = genomic RNA
aaatgttttcctgcgttgccaggaccgtccgccgctctgagtcatgtgcgagtgggaagtcgcactgacactgagccgggccagagggagaggagccgagcgcggcgcggggccgagggactcgcagtgtgtgtagagagccgggctcctgcggatgggggctgcccccggggcctgagcccgcctgcccgcccaccgccccgccccgcccctgccacccctgccgcccggttcccattagcctgtccgcctctgcgggaccatggagtggtagccgaggaggaagcatgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagaatccaggagtcctccagctgagaacgaggtgtccacccccatgcaggcacttactactaataaagacgatgataatattctcttcagagattctgcaaatgcgacaagcctcccagtgcaagattcttcttcagtaccactgcccacattcctggttgctggagggagcctggccttcggaacgctcctctgcattgccattgttctgaggttcaagaagacgtggaagctgcgggctctgaaggaaggcaagacaagcatgcatccgccgtactctttggggcagctggtcccggagaggcctcgacccaccccagtgcttgttcctctcatctccccaccggtgtcccccagcagcctggggtctgacaatacctcgagccacaaccgaccagatgccagggacccacggagcccttatgacatcagcaatacagactacttcttccccagatagctggctgggtggcaccagcagcctggaccctgtggatgataaaacacaaacgggctcagcaaaagatgcttctcactgccatgccagcttatctcaggggtgtgcggcctttggcttcacggaagagccttgcggaaggttctacgccaggggaaaatcagcctgctccagctgttcagctggttgaggtttcaaacctccctttccaaatgcccagcttaaaggggctagagtgaacttgggccactgtgaagagaaccatatcaagactctttggacactcacacggacactcaaaagctgggcaggttggtgggggcctcggtgtggagaagcggctggcagcccacccctcaacacctctgcacaagctgcaccctcaggcaggtgggatggatttccagccaaagcctcctccagccgccatgctcctggcccactgcatcgtttcatcttccaactcaaactcttaaaacccaagtgccttagcaaattctgtttttctaggcctggggacggcttttacttaaaccgccaaggctgggggaagaagctctctcctccctttcttccctacagttgaaaaacagctgagggtgagtgggtgaataatacagtatctcagggcctggtcgttttcaacagaattataattagttcctcattagcattttgctaaatgtgaatgatgatcctaggcatttgctgaatacagaggcaactgcattggctttgggttgcaggacctcaggtgagaagcagaggaaggagaggagaggggcacagggtctctaccatcccctgtagagtgggagctgagtgggggatcacagcctctgaaaaccaatgttctctcttctccacctcccacaaaggagagctagcagcagggagggcttctgccatttctgagatcaaaacggttttactgcagctttgtttgttgtcagctgaacctgggtaactagggaagataatattaaggaagacaatgtgaaaagaaaaatgagcctggcaagaatgtgtttaaacttggtttttaaaaaactgctgactgttttctcttgagagggtggaatatccaatattcgctgtgtcagcatagaagtaacttacttaggtgtgggggaagcaccataactttgtttagcccaaaaccaagtcaagtgaaaaaggaggaagagaaaaaatattttcctgccaggcatggtggcccacgcacttcgggaggtcgaggcaggaggatcacttgagtccagaagtttgagatcagcctgggcaatgtgataaaaccccatctctacaaaaagcataaaaattagccaagtgtggtagagtgtgcctgaagtcccagatacttggggggctgaggtgggaggatctcttgagcctgggaggtcaaggctgcagtgagccgagattgcaccactgcactccagcctgggtgacagagcaagtgagaccctgtctcaaaaaaagaaaaagaaaaagaaaaaatattttccctattagagaagagattgtggtttcattctgtattttgtttttgtcttaaaaagtggaaaaatagcctgcctcttctctactctagggaaaaaccagcgtgtgactactcccccaggtggttatggagagggtgtccggtccctgtcccagtgccgagaaggaagcctcccacgactgcccggcagggtcctagaaattccccaccctgaaagccctgagctttctgctatcaaagaggttttaaaaaaatcccatttaaaaaaaatcccttacctcggtgccttcctctttttatttagttccttgagttgattcagctctgcaagaattgaagcaggactaaatgtctagttgtaacaccatgattaaccacttcagctgacttttctgtccgagctttgaaaattcagtggtgttagtggttacccagttagctctcaagttatcagggtattccagagtggggatatgatttaaatcagccgtgtaaccatggacccaatatttaccagaccacaaaacttttctaatactctaccctcttagaaaaaccaccaccatcaccagacaggtgcgaaaggatgaaagtgaccatgttttgtttacggttttccaggtttaagctgttactgtcttcagtaagccgtgattttcattgctgggcttgtctgtagattttagaccctattgctgcttgaggcaactcatcttaggttggcaaaaaggcaggatggccgggcgcggtggctcacgcctgtaatcctagcactttgggaggccaaggtgggaggattgcttgagctcaggagtttgagaccaacctgggtaacatagtgagacaccatctctattatgaacaataacagttaagaaaaaaaaaggcaggcaggcggttatggtggttccctcccatcccaccacataaagtttctgagacttgagaacagcaaaatgctgttaaagggaaatattaagaatgagaatctgcagtaagggtgattctgtgcccacagttcttcaattctttataccgttttacccacatgtggtgttaccaaagccgggcagaaccatgctagcggaagatgtgaaatccagatagctcattattgccaagagctaggcagctttgatctccaaattgttattgctttcatttttattgtaatggaattgctttgttttgtttttttgtttttgtattgaagagggttgttttccctttatttttcataagctaatgtaaatgaagaaaaaatgtcttctctgggctgtaggcctggctcagcgtacacaggtatacatcctaagctctctatgttctctaatctgtggtgactgaacatgtgtctcaatgcacggggcatttctacctgtgtttctgcagcacccccactgccttgagtccccagcagtgctgttatttgcctaacacctgtagccatctgccacgcagccagacgtgaaacgctgagacagagaccatttaggttaaatacgacagcttatcctgctgggtggggaaagtaaaaaatatgctggttcaaggcctaaagtaaaatgatcaataatgtttgtagcattaatgaaatattttcaagaaatgtgtccaggggtagcactggctatgttgacgaggcctttggtaactcagagagctcttggccctgatggggacttgcccttacgctttctttatcaggctctgagttcacacggagcctctggcacttccctgctgtcttgggagaaaggaaactggttgccgcggcaggttgtggaatctgttgctggaaccaggctggaagcccacctggtagtgaacagggcccagtggggcaggctgggcatgttgtggtctatgggtttgtttcctggagaatgttcaggaatgtcttcccagctgctttggtgctgagctctattatctcacagcacgtccagaaggctaacccaggtggggaggatgctgacaccagctccaggtggagttggtggtcttaatttggagatgcaggggcaacctgtgaccctttgaggcaagagccctgcacccagctgtcccgtgcagccgtgggcaggggctgcacacggaggggcaggcgggccagttcagggtccgtgccaggccctcctcagtgccctgtgaaggcctcctgtcctccgtgcggctgggcaccagcaccagggagtttctatggcaaccttagtgattattaaggaacactgtcagttttatgaacatatgctcaaatgaaattctactttaggaggaaaggattggaacagcatgtcacaaggctgttaattaacagagagaccttattggatggagatcacatctgttaaatagaatacctcaactctacgttgttttcttggagataaataatagtttcaagtttttgtttgtttgttttacctaattacctgaaagcaaataccaaaggctgatgtctgtatatggggcaaagggtcagtatatttttcagtgtttttttttctaccagctattttgcatttaaagtgaacattgtgtttggaataaatactcttaaaaaata

SEQ ID NO: 9 - RNA, 5670 nt, Homo sapiens
  source 1..5670: mol_type = genomic RNA
aaatgttttcctgcgttgccaggaccgtccgccgctctgagtcatgtgcgagtgggaagtcgcactgacactgagccgggccagagggagaggagccgagcgcggcgcggggccgagggactcgcagtgtgtgtagagagccgggctcctgcggatgggggctgcccccggggcctgagcccgcctgcccgcccaccgccccgccccgcccctgccacccctgccgcccggttcccattagcctgtccgcctctgcgggaccatggagtggtagccgaggaggaagcatgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagaatccaggagtcctccagctgagaacgaggtgtccacccccatgcaggcacttactactaataaagacgatgataatattctcttcagagattctgcaaatgcgacaagcctcccaggttcaagaagacgtggaagctgcgggctctgaaggaaggcaagacaagcatgcatccgccgtactctttggggcagctggtcccggagaggcctcgacccaccccagtgcttgttcctctcatctccccaccggtgtcccccagcagcctggggtctgacaatacctcgagccacaaccgaccagatgccagggacccacggagcccttatgacatcagcaatacagactacttcttccccagatagctggctgggtggcaccagcagcctggaccctgtggatgataaaacacaaacgggctcagcaaaagatgcttctcactgccatgccagcttatctcaggggtgtgcggcctttggcttcacggaagagccttgcggaaggttctacgccaggggaaaatcagcctgctccagctgttcagctggttgaggtttcaaacctccctttccaaatgcccagcttaaaggggctagagtgaacttgggccactgtgaagagaaccatatcaagactctttggacactcacacggacactcaaaagctgggcaggttggtgggggcctcggtgtggagaagcggctggcagcccacccctcaacacctctgcacaagctgcaccctcaggcaggtgggatggatttccagccaaagcctcctccagccgccatgctcctggcccactgcatcgtttcatcttccaactcaaactcttaaaacccaagtgccttagcaaattctgtttttctaggcctggggacggcttttacttaaaccgccaaggctgggggaagaagctctctcctccctttcttccctacagttgaaaaacagctgagggtgagtgggtgaataatacagtatctcagggcctggtcgttttcaacagaattataattagttcctcattagcattttgctaaatgtgaatgatgatcctaggcatttgctgaatacagaggcaactgcattggctttgggttgcaggacctcaggtgagaagcagaggaaggagaggagaggggcacagggtctctaccatcccctgtagagtgggagctgagtgggggatcacagcctctgaaaaccaatgttctctcttctccacctcccacaaaggagagctagcagcagggagggcttctgccatttctgagatcaaaacggttttactgcagctttgtttgttgtcagctgaacctgggtaactagggaagataatattaaggaagacaatgtgaaaagaaaaatgagcctggcaagaatgtgtttaaacttggtttttaaaaaactgctgactgttttctcttgagagggtggaatatccaatattcgctgtgtcagcatagaagtaacttacttaggtgtgggggaagcaccataactttgtttagcccaaaaccaagtcaagtgaaaaaggaggaagagaaaaaatattttcctgccaggcatggtggcccacgcacttcgggaggtcgaggcaggaggatcacttgagtccagaagtttgagatcagcctgggcaatgtgataaaaccccatctctacaaaaagcataaaaattagccaagtgtggtagagtgtgcctgaagtcccagatacttggggggctgaggtgggaggatctcttgagcctgggaggtcaaggctgcagtgagccgagattgcaccactgcactccagcctgggtgacagagcaagtgagaccctgtctcaaaaaaagaaaaagaaaaagaaaaaatattttccctattagagaagagattgtggtttcattctgtattttgtttttgtcttaaaaagtggaaaaatagcctgcctcttctctactctagggaaaaaccagcgtgtgactactcccccaggtggttatggagagggtgtccggtccctgtcccagtgccgagaaggaagcctcccacgactgcccggcagggtcctagaaattccccaccctgaaagccctgagctttctgctatcaaagaggttttaaaaaaatcccatttaaaaaaaatcccttacctcggtgccttcctctttttatttagttccttgagttgattcagctctgcaagaattgaagcaggactaaatgtctagttgtaacaccatgattaaccacttcagctgacttttctgtccgagctttgaaaattcagtggtgttagtggttacccagttagctctcaagttatcagggtattccagagtggggatatgatttaaatcagccgtgtaaccatggacccaatatttaccagaccacaaaacttttctaatactctaccctcttagaaaaaccaccaccatcaccagacaggtgcgaaaggatgaaagtgaccatgttttgtttacggttttccaggtttaagctgttactgtcttcagtaagccgtgattttcattgctgggcttgtctgtagattttagaccctattgctgcttgaggcaactcatcttaggttggcaaaaaggcaggatggccgggcgcggtggctcacgcctgtaatcctagcactttgggaggccaaggtgggaggattgcttgagctcaggagtttgagaccaacctgggtaacatagtgagacaccatctctattatgaacaataacagttaagaaaaaaaaaggcaggcaggcggttatggtggttccctcccatcccaccacataaagtttctgagacttgagaacagcaaaatgctgttaaagggaaatattaagaatgagaatctgcagtaagggtgattctgtgcccacagttcttcaattctttataccgttttacccacatgtggtgttaccaaagccgggcagaaccatgctagcggaagatgtgaaatccagatagctcattattgccaagagctaggcagctttgatctccaaattgttattgctttcatttttattgtaatggaattgctttgttttgtttttttgtttttgtattgaagagggttgttttccctttatttttcataagctaatgtaaatgaagaaaaaatgtcttctctgggctgtaggcctggctcagcgtacacaggtatacatcctaagctctctatgttctctaatctgtggtgactgaacatgtgtctcaatgcacggggcatttctacctgtgtttctgcagcacccccactgccttgagtccccagcagtgctgttatttgcctaacacctgtagccatctgccacgcagccagacgtgaaacgctgagacagagaccatttaggttaaatacgacagcttatcctgctgggtggggaaagtaaaaaatatgctggttcaaggcctaaagtaaaatgatcaataatgtttgtagcattaatgaaatattttcaagaaatgtgtccaggggtagcactggctatgttgacgaggcctttggtaactcagagagctcttggccctgatggggacttgcccttacgctttctttatcaggctctgagttcacacggagcctctggcacttccctgctgtcttgggagaaaggaaactggttgccgcggcaggttgtggaatctgttgctggaaccaggctggaagcccacctggtagtgaacagggcccagtggggcaggctgggcatgttgtggtctatgggtttgtttcctggagaatgttcaggaatgtcttcccagctgctttggtgctgagctctattatctcacagcacgtccagaaggctaacccaggtggggaggatgctgacaccagctccaggtggagttggtggtcttaatttggagatgcaggggcaacctgtgaccctttgaggcaagagccctgcacccagctgtcccgtgcagccgtgggcaggggctgcacacggaggggcaggcgggccagttcagggtccgtgccaggccctcctcagtgccctgtgaaggcctcctgtcctccgtgcggctgggcaccagcaccagggagtttctatggcaaccttagtgattattaaggaacactgtcagttttatgaacatatgctcaaatgaaattctactttaggaggaaaggattggaacagcatgtcacaaggctgttaattaacagagagaccttattggatggagatcacatctgttaaatagaatacctcaactctacgttgttttcttggagataaataatagtttcaagtttttgtttgtttgttttacctaattacctgaaagcaaataccaaaggctgatgtctgtatatggggcaaagggtcagtatatttttcagtgtttttttttctaccagctattttgcatttaaagtgaacattgtgtttggaataaatactcttaaaaaata

SEQ ID NO: 10 - RNA, 1887 nt, Homo sapiens
  source 1..1887: mol_type = genomic RNA
aaatgttttcctgcgttgccaggaccgtccgccgctctgagtcatgtgcgagtgggaagtcgcactgacactgagccgggccagagggagaggagccgagcgcggcgcggggccgagggactcgcagtgtgtgtagagagccgggctcctgcggatgggggctgcccccggggcctgagcccgcctgcccgcccaccgccccgccccgcccctgccacccctgccgcccggttcccattagcctgtccgcctctgcgggaccatggagtggtagccgaggaggaagcatgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagacaggctttctcctcgttgcccaggatggagtacagcagtgcaatcacagctcacggcaacttctgcctcctgggttcaagcaatcctcccgcctcagcctcctaagtagctgggaccacaggcgtgtgccacaatgctaattttttaaaaatgttttgtagagacagggtttcaccatgctgcccaggctggtctcgaactcctggcctcaagtgatccaccagcctcagactcccaaagtgctgggattactggtgtgagccactgcacctgactaaactttaaatttttttttttagacggaatctcgctctgttgcccaggctggagtgcagtggcatgatattggctcactgcaagctctgcctcttgggttcacgctattctcctgcctcagcctcctgagtagctgggactacaggtgcacaccaccacgcccggctaatttttttttttttttagtagagacggggtttcactgtgttggccaggctggtcttgaactcctgacctcgtgatccacccgcctcgccctcccaaaatgctgggattacaggtgtgagccaccgcgcctggcctaaacttttaaaattttaatcaaattaatacatgcacatggcaaagaagtaataaacagcttataacactga

SEQ ID NO: 11 - RNA, 2171 nt, Homo sapiens
  source 1..2171: mol_type = genomic RNA
actcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaa

SEQ ID NO: 12 - DNA, 21 bp, synthetic construct
  source 1..21: mol_type = other DNA
  modified_base 21: mod_base = OTHER; note = Dibenzocyclooctyne modified nucleotide
  misc_feature 1..19: note = RNA
  misc_feature 20..21: note = DNA
gactcaagatggaacctgatt

SEQ ID NO: 13 - DNA, 21 bp, synthetic construct
  source 1..21: mol_type = other DNA
  modified_base 21: mod_base = OTHER; note = Cy5.5 modified nucleotide
  misc_feature 1..2: note = DNA
  misc_feature 3..21: note = RNA
ttctgagttctaccttggact

SEQ ID NO: 14 - RNA, 3687 nt, Homo sapiens
  source 1..3687: mol_type = genomic RNA
agtcgagggctagcgagcgcagcggagcctggagagaaggcgctgggctgcgagggcgcgagggcgcgagggcagggggcaaccggaccccgcccgcacccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaaccaggccggtgtgggctgtgtcgtagccaaggtgggctgagccctggcaggatgaccctgcgaaggggccctggtccttccaggcccccaccactaggactctgaggctctttctgggccaagttcctctagtgccctccacagccgcagcctccctctgacctgcaggccaagagcagaggcagcgagttgtggaaagcctctgctgccatggcgtgtccctctcggaaggctggctgggcatggacgttcggggcatgctggggcaagtccctgactctctgtgacctgccccgcccagctgcacctgccagcctggcttctggagcccttgggttttttgtttgtttgtttgtttgtttgtttgtttctccccctgggctctgccccagctctggcttccagaaaaccccagcatccttttctgcagaggggctttctggagaggagggatgctgcctgagtcacccatgaagacaggacagtgcttcagcctgaggctgagactgcgggatggtcctggggctctgtgcagggaggaggtggcagccctgtagggaacggggtccttcaagttagctcaggaggcttggaaagcatcacctcaggccaggtgcagtggctcacgcctatgatcccagcactttgggaggctgaggcgggtggatcacctgaggttaggagttcgagaccagcctggccaacatggtaaaaccccatctctactaaaaatacagaaattagccgggcgtggtggcgggcacctatagtcccagctactcagaagcctgaggctgggaaatcgtttgaacccgggaagcggaggttgcagggagccgagatcacgccactgcactccagcctgggcgacagagcgagagtctgtctcaaaagaaaaaaaaaagcaccgcctccaaatgccaacttgtccttttgtaccatggtgtgaaagtcagatgcccagagggcccaggcaggccaccatattcagtgctgtggcctgggcaagataacgcacttctaactagaaatctgccaattttttaaaaaagtaagtaccactcaggccaacaagccaacgacaaagccaaactctgccagccacatccaaccccccacctgccatttgcaccctccgccttcactccggtgtgcctgcagccccgcgcctccttccttgctgtcctaggccacaccatctcctttcagggaatttcaggaactagagatgactgagtcctcgtagccatctctctactcctacctcagcctagaccctcctcctcccccagaggggtgggttcctcttccccactccccaccttcaattcctgggccccaaacgggctgccctgccactttggtacatggccagtgtgatcccaagtgccagtcttgtgtctgcgtctgtgttgcgtgtcgtgggtgtgtgtagccaaggtcggtaagttgaatggcctgccttgaagccactgaagctgggattcctccccattagagtcagccttccccctcccagggccagggccctgcagaggggaaaccagtgtagccttgcccggattctgggaggaagcaggttgaggggctcctggaaaggctcagtctcaggagcatggggataaaggagaaggcatgaaattgtctagcagagcaggggcagggtgataaattgttgataaattccactggacttgagcttggcagctgaactattggagggtgggagagcccagccattaccatggagacaagaagggttttccaccctggaatcaagatgtcagactggctggctgcagtgacgtgcacctgtactcaggaggctgaggggaggatcactggagcccaggagtttgaggctgcagcgagctatgatcgcgccactacactccagcctgagcaacagagtgagaccctgtctcttaaagaaaaaaaaagtcagactgctgggactggccaggtttctgcccacattggacccacatgaggacatgatggagcgcacctgccccctggtggacagtcctgggagaacctcaggcttccttggcatcacagggcagagccgggaagcgatgaatttggagactctgtggggccttggttcccttgtgtgtgtgtgttgatcccaagacaatgaaagtttgcactgtatgctggacggcattcctgcttatcaataaacctgtttgtttta

SEQ ID NO: 15 - RNA, 4832 nt, Homo sapiens
  source 1..4832: mol_type = genomic RNA
gaaggacgcaacgctgccaccatggacagtagcacctggagccccaagaccaccgcggtcacccggcctgttgagacccacgagctcattcgcaatgcagccgatatctccatcatcgttatctacttcgtggtagtgatggccgtcggactgtgggctatgttttccaccaatcgtgggactgttggaggcttcttcctggcaggccgaagtatggtgtggtggccgattggagcctccctctttgctagtaacattggaagtggccactttgtggggctggccgggactggggcagcttcaggcatcgccattggaggctttgaatggaatgccctggttttggtggttgtgctgggctggctgtttgtccccatctatattaaggctggggtggtgacaatgccagagtacctgaggaagcggtttggaggccagcggatccaggtctacctttcccttctgtccctgctgctctacattttcaccaagatctcggcagacatcttctcgggggccatattcatcaatctggccttaggcctgaatctgtatttagccatctttctcttattggcaatcactgccctttacacaattacagggggcctggcggcggtgatttacacggacaccttgcagacggtgatcatgctggtggggtctttaatcctgactgggtttgcttttcacgaagtgggaggctatgacgccttcatggaaaagtacatgaaagccattccaaccatagtgtctgatggcaacaccacctttcaggaaaaatgctacactccaagggccgactccttccacatcttccgagatcccctcacgggagacctcccatggcctgggttcatctttgggatgtccatccttaccttgtggtactggtgcacagatcaggtcattgtgcagcgctgcctctcagccaagaatatgtctcacgtgaagggtggctgcatcctgtgtgggtatctaaagctgatgcccatgttcatcatggtgatgccaggaatgatcagccgcattctgtacacagaaaaaattgcctgtgtcgtcccttcagaatgtgagaaatattgcggtaccaaggttggctgtaccaacatcgcctatccaaccttagtggtggagctcatgcccaatggactgcgaggcctgatgctatcagtcatgctggcctccctcatgagctccctgacctccatcttcaacagcgccagcaccctcttcaccatggacatctacgccaaggtccgcaagagagcatctgagaaagagctcatgattgccggaaggttgtttatcctggtgctgattggcatcagcatcgcctgggtgcccattgtgcagtcagcacaaagtgggcaactcttcgattacatccagtccatcaccagttacttgggaccacccattgcggctgtcttcctgcttgctattttctggaagagagtcaatgagccaggagccttttggggactgatcctaggacttctgattgggatttcacgtatgattactgagtttgcttatggaaccgggagctgcatggagcccagcaactgtcccacgattatctgtggggtgcactacttgtactttgccattatcctcttcgccatttctttcatcaccatcgtggtcatctccctcctcaccaaacccattccggatgtgcatctctaccgtctgtgttggagcctgcgcaacagcaaagaggagcgtattgacctggatgcggaagaggagaacatccaagaaggccctaaggagaccattgaaatagaaacacaagttcctgagaagaaaaaaggaatcttcaggagagcctatgacctattttgtgggctagagcagcacggtgcacccaagatgactgaggaagaggagaaagccatgaagatgaagatgacggacacctctgagaagcctttgtggaggacagtgttgaacgtcaatggcatcatcctggtgaccgtggctgtcttttgccatgcatattttgcctgagtcctaccttttgctgtagatttaccatggctggactcttactcaccttcctttagtctcgtcctgtggtgttgaagggaaatcagccagttgtaaattttgcccaggtggataaatgtgtacatgtgtaattataggctagctggaagaaaaccattagtttgctgttaatttatgcatttgaagccagtgtgatacagccatctgtacctactggagctgcagaagggaagtccactcagtcacatccagaaaaaggcagactaagaatcagaagccatgtgattgatgtctgacgtgagtctgtctcaggtagattccgggtgtcagtgtggtttataatccttgaatattgttttagaaactttggtctccctggttcctgccacttttcctgtccgtcctcctccccattttttttttaaaagaaagctgttttcccctcatcatatccctcttgagttttgcctggactttccctctcaagtgtgtcaatcaggtaaactgaggaatgcatggaagctgaggatggagcttgatgggctccctgtcctgggtgtttgctctctgaagtggaggcctgaggaaggtagtacttccacaaaagggagggacccgggccccagcctcaagctagtgggggaggcagatagcctgaatccaggggattttctgggcttcttaaaatgtccattgtgagttccccgtgtttgggattccactcattttggcattcacagtgcctggaatgtcttagattttcagcaatgcgtgttgaataaatgaatgacataggcatttatttttaaatctttgcttgctttttacatgagcctggcccttagttaaccttttcttgtggctacacaaagtatgctcactggttactaatgacttgggatgcatttgtcaaactgattatattagttttctagggatgccataacaaagtagcacagaccagatggctcaagcagcagacatttattttctcacagttctagaggctagaagttggaggccaagatgtcagcagggttggtttcttctgaggcctctctccttggttgcagatggtcatatctcactctgtcttccgtggccttccttttgtctgtgtcctaaatctactcttctgataaggacatcagtcatattggaataggacccaccctaatgtcttcattttaatcacctctttaaagcccctacctccaaatacagtcacactgtgagaaactgagggttaggaagtcagcaagtgagtcttgaagagatactaaacaaacccacaacacagataaagtatgcattttggagatttccaagccagagtctcccgtgaaaaaggtaaacggaagcagttattgtgcagcaaaaggaaaaagaattacaaactgaacgtatgtaggtgaggcaaggcagggtagggcagggcctttgggtaggctgatcagagggtttttcaacaataaatcaatgggaatgcatttgttgctcccaggaccctggcaccttgactctggtactatagcatgtcagcaaatacaagcaaagcccaacactctgatttgcatttatgccaatctaaactatccggtgtttagtttgattttttgagtgcaggttcattcaaggaccaggttcccttgtgctcagggtgaagtagaaccagaaaacatcgttatccattcccagaagttttggaagagccttggtagaaaagcagaagctgctttgaccgtgaaaatatttgactcctatcagtttttggtcaggagaagatatccacctagaccaacctgaggagaaggctcagagtacagatataccccgagcaacgtgatcaatgtccttgaaccttcatttttcatctgaaaacagagacataaatgcctggctcacagatttaaatgttatacattgacagcatttatcagtataacatttatttaaataagtaggtgctcaataggtgttggtcttctaacttgtctacatcccatccccattccagggtcttcagaattgaaggagagatgttgtatcactgttagaaggctgctttgggacattctgcagcagggaggagggactgtcaacccctacaccatgaccaccaagttcctcaccttggctgagtccctaaaactctctgaacctcaggttcctccaagcataatgcagacttcacagagctgttgtaaagattaggtgaggtcaattgatactgcttaaaaggcccggtccgtagaaaatgcccaataaacattactgctttccccctcaccctactgcctgaaaaaatattacacctgtgagactgactttgagaaccagtgtgggtggggagttgtgcatataaactatttaatgagtaccaaacacaaaagtcaagcttgtaaaatatcaggccttgccccagaaagacaaataccacatgatctcactgatatgtagaatcttaaaaagtcaaactcagaagcagagagtagaatgatggttatcaagggctgggggagggagggactggggagatgttggtcaaatgatacaaaggtttagttaggtggaataagttcagaaaatcaattgtacaatgtatcaattatagttaatagcaatataacatatacttgaaaattgctgagagtagtgtgagtgttctaccacaaaaaaatatgtgcagtaatagatgttaattaccttaatttagtcatttcacaatatgtacatatataaaaatatgttgtatgccatgagtatatataattattatttgtgaatttaaaaaataaaaataatttccaaaaaaa

SEQ ID NO: 16 - RNA, 4811 nt, Homo sapiens
  source 1..4811: mol_type = genomic RNA
gaatttcatgccacagctgagaggcagaagttacagtgagccgagatcatgccactgcactcctgcctgggcgacagaaaaagactctgtcccaaaagtgggctctttgaaggctagaaggtatttggggcagcagacttgtgtggaagcagttatcaaagtggggatgtgacacgcaccaggagaggggaacagacaacacatgagattggagcctccctctttgctagtaacattggaagtggccactttgtggggctggccgggactggggcagcttcaggcatcgccattggaggctttgaatggaatgccctggttttggtggttgtgctgggctggctgtttgtccccatctatattaaggctggggtggtgacaatgccagagtacctgaggaagcggtttggaggccagcggatccaggtctacctttcccttctgtccctgctgctctacattttcaccaagatctcggcagacatcttctcgggggccatattcatcaatctggccttaggcctgaatctgtatttagccatctttctcttattggcaatcactgccctttacacaattacagggggcctggcggcggtgatttacacggacaccttgcagacggtgatcatgctggtggggtctttaatcctgactgggtttgcttttcacgaagtgggaggctatgacgccttcatggaaaagtacatgaaagccattccaaccatagtgtctgatggcaacaccacctttcaggaaaaatgctacactccaagggccgactccttccacatcttccgagatcccctcacgggagacctcccatggcctgggttcatctttgggatgtccatccttaccttgtggtactggtgcacagatcaggtcattgtgcagcgctgcctctcagccaagaatatgtctcacgtgaagggtggctgcatcctgtgtgggtatctaaagctgatgcccatgttcatcatggtgatgccaggaatgatcagccgcattctgtacacagaaaaaattgcctgtgtcgtcccttcagaatgtgagaaatattgcggtaccaaggttggctgtaccaacatcgcctatccaaccttagtggtggagctcatgcccaatggactgcgaggcctgatgctatcagtcatgctggcctccctcatgagctccctgacctccatcttcaacagcgccagcaccctcttcaccatggacatctacgccaaggtccgcaagagagcatctgagaaagagctcatgattgccggaaggttgtttatcctggtgctgattggcatcagcatcgcctgggtgcccattgtgcagtcagcacaaagtgggcaactcttcgattacatccagtccatcaccagttacttgggaccacccattgcggctgtcttcctgcttgctattttctggaagagagtcaatgagccaggagccttttggggactgatcctaggacttctgattgggatttcacgtatgattactgagtttgcttatggaaccgggagctgcatggagcccagcaactgtcccacgattatctgtggggtgcactacttgtactttgccattatcctcttcgccatttctttcatcaccatcgtggtcatctccctcctcaccaaacccattccggatgtgcatctctaccgtctgtgttggagcctgcgcaacagcaaagaggagcgtattgacctggatgcggaagaggagaacatccaagaaggccctaaggagaccattgaaatagaaacacaagttcctgagaagaaaaaaggaatcttcaggagagcctatgacctattttgtgggctagagcagcacggtgcacccaagatgactgaggaagaggagaaagccatgaagatgaagatgacggacacctctgagaagcctttgtggaggacagtgttgaacgtcaatggcatcatcctggtgaccgtggctgtcttttgccatgcatattttgcctgagtcctaccttttgctgtagatttaccatggctggactcttactcaccttcctttagtctcgtcctgtggtgttgaagggaaatcagccagttgtaaattttgcccaggtggataaatgtgtacatgtgtaattataggctagctggaagaaaaccattagtttgctgttaatttatgcatttgaagccagtgtgatacagccatctgtacctactggagctgcagaagggaagtccactcagtcacatccagaaaaaggcagactaagaatcagaagccatgtgattgatgtctgacgtgagtctgtctcaggtagattccgggtgtcagtgtggtttataatccttgaatattgttttagaaactttggtctccctggttcctgccacttttcctgtccgtcctcctccccattttttttttaaaagaaagctgttttcccctcatcatatccctcttgagttttgcctggactttccctctcaagtgtgtcaatcaggtaaactgaggaatgcatggaagctgaggatggagcttgatgggctccctgtcctgggtgtttgctctctgaagtggaggcctgaggaaggtagtacttccacaaaagggagggacccgggccccagcctcaagctagtgggggaggcagatagcctgaatccaggggattttctgggcttcttaaaatgtccattgtgagttccccgtgtttgggattccactcattttggcattcacagtgcctggaatgtcttagattttcagcaatgcgtgttgaataaatgaatgacataggcatttatttttaaatctttgcttgctttttacatgagcctggcccttagttaaccttttcttgtggctacacaaagtatgctcactggttactaatgacttgggatgcatttgtcaaactgattatattagttttctagggatgccataacaaagtagcacagaccagatggctcaagcagcagacatttattttctcacagttctagaggctagaagttggaggccaagatgtcagcagggttggtttcttctgaggcctctctccttggttgcagatggtcatatctcactctgtcttccgtggccttccttttgtctgtgtcctaaatctactcttctgataaggacatcagtcatattggaataggacccaccctaatgtcttcattttaatcacctctttaaagcccctacctccaaatacagtcacactgtgagaaactgagggttaggaagtcagcaagtgagtcttgaagagatactaaacaaacccacaacacagataaagtatgcattttggagatttccaagccagagtctcccgtgaaaaaggtaaacggaagcagttattgtgcagcaaaaggaaaaagaattacaaactgaacgtatgtaggtgaggcaaggcagggtagggcagggcctttgggtaggctgatcagagggtttttcaacaataaatcaatgggaatgcatttgttgctcccaggaccctggcaccttgactctggtactatagcatgtcagcaaatacaagcaaagcccaacactctgatttgcatttatgccaatctaaactatccggtgtttagtttgattttttgagtgcaggttcattcaaggaccaggttcccttgtgctcagggtgaagtagaaccagaaaacatcgttatccattcccagaagttttggaagagccttggtagaaaagcagaagctgctttgaccgtgaaaatatttgactcctatcagtttttggtcaggagaagatatccacctagaccaacctgaggagaaggctcagagtacagatataccccgagcaacgtgatcaatgtccttgaaccttcatttttcatctgaaaacagagacataaatgcctggctcacagatttaaatgttatacattgacagcatttatcagtataacatttatttaaataagtaggtgctcaataggtgttggtcttctaacttgtctacatcccatccccattccagggtcttcagaattgaaggagagatgttgtatcactgttagaaggctgctttgggacattctgcagcagggaggagggactgtcaacccctacaccatgaccaccaagttcctcaccttggctgagtccctaaaactctctgaacctcaggttcctccaagcataatgcagacttcacagagctgttgtaaagattaggtgaggtcaattgatactgcttaaaaggcccggtccgtagaaaatgcccaataaacattactgctttccccctcaccctactgcctgaaaaaatattacacctgtgagactgactttgagaaccagtgtgggtggggagttgtgcatataaactatttaatgagtaccaaacacaaaagtcaagcttgtaaaatatcaggccttgccccagaaagacaaataccacatgatctcactgatatgtagaatcttaaaaagtcaaactcagaagcagagagtagaatgatggttatcaagggctgggggagggagggactggggagatgttggtcaaatgatacaaaggtttagttaggtggaataagttcagaaaatcaattgtacaatgtatcaattatagttaatagcaatataacatatacttgaaaattgctgagagtagtgtgagtgttctaccacaaaaaaatatgtgcagtaatagatgttaattaccttaatttagtcatttcacaatatgtacatatataaaaatatgttgtatgccatgagtatatataattattatttgtgaatttaaaaaataaaaataatttccaaaaaaa

SEQ ID NO: 17 - RNA, 2267 nt, Homo sapiens
  source 1..2267: mol_type = genomic RNA
agatcctggggagaatggaggagcacacagaggcaggctcggcaccagagatgggggcccagaaggccctgattgacaatcctgctgacatcctagtcattgctgcatatttcctgctggtcattggcgttggcttgtggtccatgtgcagaaccaacagaggcactgtgggcggctacttcctggcaggacgcagcatggtgtggtggccggttggggcctctctcttcgccagcaacatcggcagtggccactttgtgggcctggcagggactggcgctgcaagtggcttggctgttgctggattcgagtggaatgcgctcttcgtggtgctgctactgggctggctgtttgcacccgtgtacctgacagcgggggtcatcacgatgccacagtacctgcgcaagcgcttcggcggccgccgcatccgcctctacctgtctgtgctctcccttttcctgtacatcttcaccaagatctcagtggacatgttctccggagctgtattcatccagcaggctctgggctggaacatctatgcctccgtcatcgcgcttctgggcatcaccatgatttacacggtgacaggagggctggccgcgctgatgtacacggacacggtacagaccttcgtcattctggggggcgcctgcatcctcatgggttacgccttccacgaggtgggcgggtattcgggtctcttcgacaaatacctgggagcagcgacttcgctgacggtgtccgaggatccagccgtgggaaacatctccagcttctgctatcgaccccggcccgactcctaccacctgctccggcaccccgtgaccggggatctgccgtggcccgcgctgctcctcggactcacaatcgtctcgggctggtactggtgcagcgaccaggtcatcgtgcagcgctgcctggccgggaagagcctgacccacatcaaggcgggctgcatcctgtgtgggtacctgaagctgacgcccatgtttctcatggtcatgccaggcatgatcagccgcattctgtacccagacgaggtggcgtgcgtggtgcctgaggtgtgcaggcgcgtgtgcggcacggaggtgggctgctccaacatcgcctacccgcggctcgtcgtgaagctcatgcccaacggtctgcgcggactcatgctggcggtcatgctggccgcgctcatgtcctcgctggcctccatcttcaacagcagcagcacgctcttcaccatggacatctacacgcgcctgcggccacgcgccggcgaccgcgagctgctgctggtgggacggctctgggtggtgttcatcgtggtagtgtcggtggcctggcttcccgtggtgcaggcggcacagggcgggcagctcttcgattacatccaggcagtctctagctacctggcaccgcccgtgtccgccgtcttcgtgctggcgctcttcgtgccgcgcgttaatgagcagggcgccttctggggactcatcgggggcctgctgatgggcctggcacgcctgattcccgagttctccttcggctcgggcagctgtgtgcagccctcggcgtgcccagctttcctctgcggcgtgcactacctctacttcgccattgtgctgttcttctgctctggcctcctcaccctcacggtctccctgtgcaccgcgcccatccccagaaagcacctccaccgcctggtcttcagtctccggcatagcaaggaggaacgggaggacctggatgctgatgagcagcaaggctcctcactccctgtacagaatgggtgcccagagagtgccatggagatgaatgagccccaggccccggcaccaagcctcttccgccagtgcctgctctggttttgtggaatgagcagaggtggggtgggcagtcctccgccccttacccaggaggaggcagcggcagcagccaggcggctggaggacatcagcgaggacccgagctgggcccgtgtggtcaacctcaatgccctgctcatgatggcagtggccgtgttcctctggggcttctatgcctaagaccaactgcgttggacaccataagccacagcctcacaggaagtgggggtgaggagcctgcggtgctccccagaaaaggggaaggggcagtggggtgagaaggtcctggctccccttctcccggccttcctctgcctggggcccactgcatctgattggcagtcacttcccatgagggcctggcccacccgctgcagttgccctaaggaaaaataaagctgcctttcccctgtc

SEQ ID NO: 18 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 19 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 20 - protein, 1233 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 21 - protein, 272 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 22 - protein, 227 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 23 - protein, 800 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 24 - protein, 810 aa, Homo sapiens
MQKDARREGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 25 - protein, 449 aa, Homo sapiens
LAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 26 - protein, 336 aa, Homo sapiens
LAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLP

SEQ ID NO: 27 - protein, 333 aa, Homo sapiens
LAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTDRLSPRCPGWSTAVQSQLTATSASWVQAILPPQPPK

SEQ ID NO: 28 - RNA, 5863 nt, Homo sapiens
  source 1..5863: mol_type = genomic RNA
aaatgttttcctgcgttgccaggaccgtccgccgctctgagtcatgtgcgagtgggaagtcgcactgacactgagccgggccagagggagaggagccgagcgcggcgcggggccgagggactcgcagtgtgtgtagagagccgggctcctgcggatgggggctgcccccggggcctgagcccgcctgcccgcccaccgccccgccccgcccctgccacccctgccgcccggttcccattagcctgtccgcctctgcgggaccatggagtggtagccgaggaggaagcatgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagaatccaggagtcctccagctgagaacgaggtgtccacccccatgcaggcacttactactaataaagacgatgataatattctcttcagagattctgcaaatgcgacaagcctcccaggaactgccatcaaggaggtccccaaagccttccgtttgttgtaccctctttgtgccttgtgggagcaggtggaatgtcaccagaaaagccactctgaaatgcaagattcttcttcagtaccactgcccacattcctggttgctggagggagcctggccttcggaacgctcctctgcattgccattgttctgaggttcaagaagacgtggaagctgcgggctctgaaggaaggcaagacaagcatgcatccgccgtactctttggggcagctggtcccggagaggcctcgacccaccccagtgcttgttcctctcatctccccaccggtgtcccccagcagcctggggtctgacaatacctcgagccacaaccgaccagatgccagggacccacggagcccttatgacatcagcaatacagactacttcttccccagatagctggctgggtggcaccagcagcctggaccctgtggatgataaaacacaaacgggctcagcaaaagatgcttctcactgccatgccagcttatctcaggggtgtgcggcctttggcttcacggaagagccttgcggaaggttctacgccaggggaaaatcagcctgctccagctgttcagctggttgaggtttcaaacctccctttccaaatgcccagcttaaaggggctagagtgaacttgggccactgtgaagagaaccatatcaagactctttggacactcacacggacactcaaaagctgggcaggttggtgggggcctcggtgtggagaagcggctggcagcccacccctcaacacctctgcacaagctgcaccctcaggcaggtgggatggatttccagccaaagcctcctccagccgccatgctcctggcccactgcatcgtttcatcttccaactcaaactcttaaaacccaagtgccttagcaaattctgtttttctaggcctggggacggcttttacttaaaccgccaaggctgggggaagaagctctctcctccctttcttccctacagttgaaaaacagctgagggtgagtgggtgaataatacagtatctcagggcctggtcgttttcaacagaattataattagttcctcattagcattttgctaaatgtgaatgatgatcctaggcatttgctgaatacagaggcaactgcattggctttgggttgcaggacctcaggtgagaagcagaggaaggagaggagaggggcacagggtctctaccatcccctgtagagtgggagctgagtgggggatcacagcctctgaaaaccaatgttctctcttctccacctcccacaaaggagagctagcagcagggagggcttctgccatttctgagatcaaaacggttttactgcagctttgtttgttgtcagctgaacctgggtaactagggaagataatattaaggaagacaatgtgaaaagaaaaatgagcctggcaagaatgtgtttaaacttggtttttaaaaaactgctgactgttttctcttgagagggtggaatatccaatattcgctgtgtcagcatagaagtaacttacttaggtgtgggggaagcaccataactttgtttagcccaaaaccaagtcaagtgaaaaaggaggaagagaaaaaatattttcctgccaggcatggtggcccacgcacttcgggaggtcgaggcaggaggatcacttgagtccagaagtttgagatcagcctgggcaatgtgataaaaccccatctctacaaaaagcataaaaattagccaagtgtggtagagtgtgcctgaagtcccagatacttggggggctgaggtgggaggatctcttgagcctgggaggtcaaggctgcagtgagccgagattgcaccactgcactccagcctgggtgacagagcaagtgagaccctgtctcaaaaaaagaaaaagaaaaagaaaaaatattttccctattagagaagagattgtggtttcattctgtattttgtttttgtcttaaaaagtggaaaaatagcctgcctcttctctactctagggaaaaaccagcgtgtgactactcccccaggtggttatggagagggtgtccggtccctgtcccagtgccgagaaggaagcctcccacgactgcccggcagggtcctagaaattccccaccctgaaagccctgagctttctgctatcaaagaggttttaaaaaaatcccatttaaaaaaaatcccttacctcggtgccttcctctttttatttagttccttgagttgattcagctctgcaagaattgaagcaggactaaatgtctagttgtaacaccatgattaaccacttcagctgacttttctgtccgagctttgaaaattcagtggtgttagtggttacccagttagctctcaagttatcagggtattccagagtggggatatgatttaaatcagccgtgtaaccatggacccaatatttaccagaccacaaaacttttctaatactctaccctcttagaaaaaccaccaccatcaccagacaggtgcgaaaggatgaaagtgaccatgttttgtttacggttttccaggtttaagctgttactgtcttcagtaagccgtgattttcattgctgggcttgtctgtagattttagaccctattgctgcttgaggcaactcatcttaggttggcaaaaaggcaggatggccgggcgcggtggctcacgcctgtaatcctagcactttgggaggccaaggtgggaggattgcttgagctcaggagtttgagaccaacctgggtaacatagtgagacaccatctctattatgaacaataacagttaagaaaaaaaaaggcaggcaggcggttatggtggttccctcccatcccaccacataaagtttctgagacttgagaacagcaaaatgctgttaaagggaaatattaagaatgagaatctgcagtaagggtgattctgtgcccacagttcttcaattctttataccgttttacccacatgtggtgttaccaaagccgggcagaaccatgctagcggaagatgtgaaatccagatagctcattattgccaagagctaggcagctttgatctccaaattgttattgctttcatttttattgtaatggaattgctttgttttgtttttttgtttttgtattgaagagggttgttttccctttatttttcataagctaatgtaaatgaagaaaaaatgtcttctctgggctgtaggcctggctcagcgtacacaggtatacatcctaagctctctatgttctctaatctgtggtgactgaacatgtgtctcaatgcacggggcatttctacctgtgtttctgcagcacccccactgccttgagtccccagcagtgctgttatttgcctaacacctgtagccatctgccacgcagccagacgtgaaacgctgagacagagaccatttaggttaaatacgacagcttatcctgctgggtggggaaagtaaaaaatatgctggttcaaggcctaaagtaaaatgatcaataatgtttgtagcattaatgaaatattttcaagaaatgtgtccaggggtagcactggctatgttgacgaggcctttggtaactcagagagctcttggccctgatggggacttgcccttacgctttctttatcaggctctgagttcacacggagcctctggcacttccctgctgtcttgggagaaaggaaactggttgccgcggcaggttgtggaatctgttgctggaaccaggctggaagcccacctggtagtgaacagggcccagtggggcaggctgggcatgttgtggtctatgggtttgtttcctggagaatgttcaggaatgtcttcccagctgctttggtgctgagctctattatctcacagcacgtccagaaggctaacccaggtggggaggatgctgacaccagctccaggtggagttggtggtcttaatttggagatgcaggggcaacctgtgaccctttgaggcaagagccctgcacccagctgtcccgtgcagccgtgggcaggggctgcacacggaggggcaggcgggccagttcagggtccgtgccaggccctcctcagtgccctgtgaaggcctcctgtcctccgtgcggctgggcaccagcaccagggagtttctatggcaaccttagtgattattaaggaacactgtcagttttatgaacatatgctcaaatgaaattctactttaggaggaaaggattggaacagcatgtcacaaggctgttaattaacagagagaccttattggatggagatcacatctgttaaatagaatacctcaactctacgttgttttcttggagataaataatagtttcaagtttttgtttgtttgttttacctaattacctgaaagcaaataccaaaggctgatgtctgtatatggggcaaagggtcagtatatttttcagtgtttttttttctaccagctattttgcatttaaagtgaacattgtgtttggaataaatactcttaaaaaata

SEQ ID NO: 29 - protein, 490 aa, Homo sapiens
LLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPGTAIKEVPKAFRLLYPLCALWEQVECHQKSHSEMQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 30 - RNA, 5857 nt, Homo sapiens
  source 1..5857: mol_type = genomic RNA
aaatgttttcctgcgttgccaggaccgtccgccgctctgagtcatgtgcgagtgggaagtcgcactgacactgagccgggccagagggagaggagccgagcgcggcgcggggccgagggactcgcagtgtgtgtagagagccgggctcctgcggatgggggctgcccccggggcctgagcccgcctgcccgcccaccgccccgccccgcccctgccacccctgccgcccggttcccattagcctgtccgcctctgcgggaccatggagtggtagccgaggaggaagcatgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagagatgaggtctcactatgttacccaggctggtttcaaactcctggcctcatgggattctccagcctcagtctcccaaagtgctgggattacagaatccaggagtcctccagctgagaacgaggtgtccacccccatgcaggcacttactactaataaagacgatgataatattctcttcagagattctgcaaatgcgacaagcctcccagtgcaagattcttcttcagtaccactgcccacattcctggttgctggagggagcctggccttcggaacgctcctctgcattgccattgttctgaggttcaagaagacgtggaagctgcgggctctgaaggaaggcaagacaagcatgcatccgccgtactctttggggcagctggtcccggagaggcctcgacccaccccagtgcttgttcctctcatctccccaccggtgtcccccagcagcctggggtctgacaatacctcgagccacaaccgaccagatgccagggacccacggagcccttatgacatcagcaatacagactacttcttccccagatagctggctgggtggcaccagcagcctggaccctgtggatgataaaacacaaacgggctcagcaaaagatgcttctcactgccatgccagcttatctcaggggtgtgcggcctttggcttcacggaagagccttgcggaaggttctacgccaggggaaaatcagcctgctccagctgttcagctggttgaggtttcaaacctccctttccaaatgcccagcttaaaggggctagagtgaacttgggccactgtgaagagaaccatatcaagactctttggacactcacacggacactcaaaagctgggcaggttggtgggggcctcggtgtggagaagcggctggcagcccacccctcaacacctctgcacaagctgcaccctcaggcaggtgggatggatttccagccaaagcctcctccagccgccatgctcctggcccactgcatcgtttcatcttccaactcaaactcttaaaacccaagtgccttagcaaattctgtttttctaggcctggggacggcttttacttaaaccgccaaggctgggggaagaagctctctcctccctttcttccctacagttgaaaaacagctgagggtgagtgggtgaataatacagtatctcagggcctggtcgttttcaacagaattataattagttcctcattagcattttgctaaatgtgaatgatgatcctaggcatttgctgaatacagaggcaactgcattggctttgggttgcaggacctcaggtgagaagcagaggaaggagaggagaggggcacagggtctctaccatcccctgtagagtgggagctgagtgggggatcacagcctctgaaaaccaatgttctctcttctccacctcccacaaaggagagctagcagcagggagggcttctgccatttctgagatcaaaacggttttactgcagctttgtttgttgtcagctgaacctgggtaactagggaagataatattaaggaagacaatgtgaaaagaaaaatgagcctggcaagaatgtgtttaaacttggtttttaaaaaactgctgactgttttctcttgagagggtggaatatccaatattcgctgtgtcagcatagaagtaacttacttaggtgtgggggaagcaccataactttgtttagcccaaaaccaagtcaagtgaaaaaggaggaagagaaaaaatattttcctgccaggcatggtggcccacgcacttcgggaggtcgaggcaggaggatcacttgagtccagaagtttgagatcagcctgggcaatgtgataaaaccccatctctacaaaaagcataaaaattagccaagtgtggtagagtgtgcctgaagtcccagatacttggggggctgaggtgggaggatctcttgagcctgggaggtcaaggctgcagtgagccgagattgcaccactgcactccagcctgggtgacagagcaagtgagaccctgtctcaaaaaaagaaaaagaaaaagaaaaaatattttccctattagagaagagattgtggtttcattctgtattttgtttttgtcttaaaaagtggaaaaatagcctgcctcttctctactctagggaaaaaccagcgtgtgactactcccccaggtggttatggagagggtgtccggtccctgtcccagtgccgagaaggaagcctcccacgactgcccggcagggtcctagaaattccccaccctgaaagccctgagctttctgctatcaaagaggttttaaaaaaatcccatttaaaaaaaatcccttacctcggtgccttcctctttttatttagttccttgagttgattcagctctgcaagaattgaagcaggactaaatgtctagttgtaacaccatgattaaccacttcagctgacttttctgtccgagctttgaaaattcagtggtgttagtggttacccagttagctctcaagttatcagggtattccagagtggggatatgatttaaatcagccgtgtaaccatggacccaatatttaccagaccacaaaacttttctaatactctaccctcttagaaaaaccaccaccatcaccagacaggtgcgaaaggatgaaagtgaccatgttttgtttacggttttccaggtttaagctgttactgtcttcagtaagccgtgattttcattgctgggcttgtctgtagattttagaccctattgctgcttgaggcaactcatcttaggttggcaaaaaggcaggatggccgggcgcggtggctcacgcctgtaatcctagcactttgggaggccaaggtgggaggattgcttgagctcaggagtttgagaccaacctgggtaacatagtgagacaccatctctattatgaacaataacagttaagaaaaaaaaaggcaggcaggcggttatggtggttccctcccatcccaccacataaagtttctgagacttgagaacagcaaaatgctgttaaagggaaatattaagaatgagaatctgcagtaagggtgattctgtgcccacagttcttcaattctttataccgttttacccacatgtggtgttaccaaagccgggcagaaccatgctagcggaagatgtgaaatccagatagctcattattgccaagagctaggcagctttgatctccaaattgttattgctttcatttttattgtaatggaattgctttgttttgtttttttgtttttgtattgaagagggttgttttccctttatttttcataagctaatgtaaatgaagaaaaaatgtcttctctgggctgtaggcctggctcagcgtacacaggtatacatcctaagctctctatgttctctaatctgtggtgactgaacatgtgtctcaatgcacggggcatttctacctgtgtttctgcagcacccccactgccttgagtccccagcagtgctgttatttgcctaacacctgtagccatctgccacgcagccagacgtgaaacgctgagacagagaccatttaggttaaatacgacagcttatcctgctgggtggggaaagtaaaaaatatgctggttcaaggcctaaagtaaaatgatcaataatgtttgtagcattaatgaaatattttcaagaaatgtgtccaggggtagcactggctatgttgacgaggcctttggtaactcagagagctcttggccctgatggggacttgcccttacgctttctttatcaggctctgagttcacacggagcctctggcacttccctgctgtcttgggagaaaggaaactggttgccgcggcaggttgtggaatctgttgctggaaccaggctggaagcccacctggtagtgaacagggcccagtggggcaggctgggcatgttgtggtctatgggtttgtttcctggagaatgttcaggaatgtcttcccagctgctttggtgctgagctctattatctcacagcacgtccagaaggctaacccaggtggggaggatgctgacaccagctccaggtggagttggtggtcttaatttggagatgcaggggcaacctgtgaccctttgaggcaagagccctgcacccagctgtcccgtgcagccgtgggcaggggctgcacacggaggggcaggcgggccagttcagggtccgtgccaggccctcctcagtgccctgtgaaggcctcctgtcctccgtgcggctgggcaccagcaccagggagtttctatggcaaccttagtgattattaaggaacactgtcagttttatgaacatatgctcaaatgaaattctactttaggaggaaaggattggaacagcatgtcacaaggctgttaattaacagagagaccttattggatggagatcacatctgttaaatagaatacctcaactctacgttgttttcttggagataaataatagtttcaagtttttgtttgtttgttttacctaattacctgaaagcaaataccaaaggctgatgtctgtatatggggcaaagggtcagtatatttttcagtgtttttttttctaccagctattttgcatttaaagtgaacattgtgtttggaataaatactcttaaaaaata

SEQ ID NO: 31 - protein, 488 aa, Homo sapiens
LLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTEMRSHYVTQAGFKLLASWDSPASVSQSAGITESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 32 - protein, 426 aa, Homo sapiens
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 33 - DNA, 20 bp, synthetic construct
  source 1..20: mol_type = other DNA
ccacccagaagactgtggat

SEQ ID NO: 34 - DNA, 20 bp, synthetic construct
  source 1..20: mol_type = other DNA
cacattgggggtaggaacac

SEQ ID NO: 35 - protein, 439 aa, Homo sapiens
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 36 - protein, 664 aa, Homo sapiens
MDSSTWSPKTTAVTRPVETHELIRNAADISIIVIYFVVVMAVGLWAMFSTNRGTVGGFFLAGRSMVWWPIGASLFASNIGSGHFVGLAGTGAASGIAIGGFEWNALVLVVVLGWLFVPIYIKAGVVTMPEYLRKRFGGQRIQVYLSLLSLLLYIFTKISADIFSGAIFINLALGLNLYLAIFLLLAITALYTITGGLAAVIYTDTLQTVIMLVGSLILTGFAFHEVGGYDAFMEKYMKAIPTIVSDGNTTFQEKCYTPRADSFHIFRDPLTGDLPWPGFIFGMSILTLWYWCTDQVIVQRCLSAKNMSHVKGGCILCGYLKLMPMFIMVMPGMISRILYTEKIACVVPSECEKYCGTKVGCTNIAYPTLVVELMPNGLRGLMLSVMLASLMSSLTSIFNSASTLFTMDIYAKVRKRASEKELMIAGRLFILVLIGISIAWVPIVQSAQSGQLFDYIQSITSYLGPPIAAVFLLAIFWKRVNEPGAFWGLILGLLIGISRMITEFAYGTGSCMEPSNCPTIICGVHYLYFAIILFAISFITIVVISLLTKPIPDVHLYRLCWSLRNSKEERIDLDAEEENIQEGPKETIEIETQVPEKKKGIFRRAYDLFCGLEQHGAPKMTEEEEKAMKMKMTDTSEKPLWRTVLNVNGIILVTVAVFCHAYFA

SEQ ID NO: 37 - protein, 537 aa, Homo sapiens
MPEYLRKRFGGQRIQVYLSLLSLLLYIFTKISADIFSGAIFINLALGLNLYLAIFLLLAITALYTITGGLAAVIYTDTLQTVIMLVGSLILTGFAFHEVGGYDAFMEKYMKAIPTIVSDGNTTFQEKCYTPRADSFHIFRDPLTGDLPWPGFIFGMSILTLWYWCTDQVIVQRCLSAKNMSHVKGGCILCGYLKLMPMFIMVMPGMISRILYTEKIACVVPSECEKYCGTKVGCTNIAYPTLVVELMPNGLRGLMLSVMLASLMSSLTSIFNSASTLFTMDIYAKVRKRASEKELMIAGRLFILVLIGISIAWVPIVQSAQSGQLFDYIQSITSYLGPPIAAVFLLAIFWKRVNEPGAFWGLILGLLIGISRMITEFAYGTGSCMEPSNCPTIICGVHYLYFAIILFAISFITIVVISLLTKPIPDVHLYRLCWSLRNSKEERIDLDAEEENIQEGPKETIEIETQVPEKKKGIFRRAYDLFCGLEQHGAPKMTEEEEKAMKMKMTDTSEKPLWRTVLNVNGIILVTVAVFCHAYFA

SEQ ID NO: 38 - protein, 672 aa, Homo sapiens
MEEHTEAGSAPEMGAQKALIDNPADILVIAAYFLLVIGVGLWSMCRTNRGTVGGYFLAGRSMVWWPVGASLFASNIGSGHFVGLAGTGAASGLAVAGFEWNALFVVLLLGWLFAPVYLTAGVITMPQYLRKRFGGRRIRLYLSVLSLFLYIFTKISVDMFSGAVFIQQALGWNIYASVIALLGITMIYTVTGGLAALMYTDTVQTFVILGGACILMGYAFHEVGGYSGLFDKYLGAATSLTVSEDPAVGNISSFCYRPRPDSYHLLRHPVTGDLPWPALLLGLTIVSGWYWCSDQVIVQRCLAGKSLTHIKAGCILCGYLKLTPMFLMVMPGMISRILYPDEVACVVPEVCRRVCGTEVGCSNIAYPRLVVKLMPNGLRGLMLAVMLAALMSSLASIFNSSSTLFTMDIYTRLRPRAGDRELLLVGRLWVVFIVVVSVAWLPVVQAAQGGQLFDYIQAVSSYLAPPVSAVFVLALFVPRVNEQGAFWGLIGGLLMGLARLIPEFSFGSGSCVQPSACPAFLCGVHYLYFAIVLFFCSGLLTLTVSLCTAPIPRKHLHRLVFSLRHSKEEREDLDADEQQGSSLPVQNGCPESAMEMNEPQAPAPSLFRQCLLWFCGMSRGGVGSPPPLTQEEAAAAARRLEDISEDPSWARVVNLNALLMMAVAVFLWGFYA

SEQ ID NO: 39 - RNA, 69 nt, Homo sapiens
  source 1..69: mol_type = genomic RNA
ctgtggtgcattgtagttgcattgcatgttctggtggtacccatgcaatgtttccacagtgcatcacag

SEQ ID NO: 40 - protein, 16 aa, synthetic construct
YRCMISYGGADYKCIT

SEQ ID NO: 41 - protein, 47 aa, synthetic construct
IQIREYKRCGQDEERVRRECKERGERQNCHYVIHKEGNCYVCGIICL

SEQ ID NO: 42 - RNA, 88 nt, Homo sapiens
  source 1..88: mol_type = genomic RNA
aactgccctcaaggagcttacaatctagctgggggtaaatgacttgcacatgaacacaactagactgtgagcttctagagggcaggga

SEQ ID NO: 43 - RNA, 77 nt, Homo sapiens
  source 1..77: mol_type = genomic RNA
gtggcctcgttcaagtaatccaggataggctgtgcaggtcccaatgggcctattcttggttacttgcacggggacgc

SEQ ID NO: 44 - RNA, 84 nt, Homo sapiens
  source 1..84: mol_type = genomic RNA
ggctgtggctggattcaagtaatccaggataggctgtttccatctgtgaggcctattcttgattacttgtttctggaggcagct

SEQ ID NO: 45 - protein, 14 aa, synthetic construct
ARGDYYGSNSLDYW

SEQ ID NO: 46 - protein, 6 aa, synthetic construct
KCCYSL

SEQ ID NO: 47 - protein, 16 aa, synthetic construct
MEGPSKCCYSLALASH

SEQ ID NO: 48 - protein, 14 aa, synthetic construct
GTKSKCCYSLRRSS

SEQ ID NO: 49 - protein, 7 aa, synthetic construct
LTVSPWY

SEQ ID NO: 50 - protein, 11 aa, synthetic construct
CGGGLTVSPWY

SEQ ID NO: 51 - protein, 11 aa, synthetic construct
CSSSLTVSPWY

SEQ ID NO: 52 - protein, 12 aa, synthetic construct
FCGDFYACYMDV

SEQ ID NO: 53 - protein, 8 aa, synthetic construct
YLFFVFER

SEQ ID NO: 54 - protein, 9 aa, synthetic construct
QDVNTAVAW

SEQ ID NO: 55 - protein, 17 aa, synthetic construct
CDTFPYLGWWNPNEYRY

SEQ ID NO: 56 - protein, 17 aa, synthetic construct
CKTIYYLGYYNPNEYRY

SEQ ID NO: 57 - protein, 19 aa, synthetic construct
GGCAKIFGSLAFLKCCYSL

SEQ ID NO: 58 - protein, 12 aa, synthetic construct
YHWYGYTPQNVI

SEQ ID NO: 59 - protein, 27 aa, synthetic construct
NKFNKGMRGYWGALGGGNGKRGIRGYD

SEQ ID NO: 60 - protein, 12 aa, synthetic construct
  SITE 1: note = Azidoacetic acid modified residue
NYSKPTDRQYHF

SEQ ID NO: 61 - protein, 9 aa, synthetic construct
CLQKTPKQC

SEQ ID NO: 62 - protein, 7 aa, synthetic construct
CVRARTR

SEQ ID NO: 63 - protein, 25 aa, synthetic construct
GSGSGSTYLCGAISLAPKAQIKESL

SEQ ID NO: 64 - protein, 12 aa, synthetic construct
WHRSYYTWNLNT

SEQ ID NO: 65 - protein, 12 aa, synthetic construct
WHFSYNWRWLPP

SEQ ID NO: 66 - protein, 9 aa, synthetic construct
CRKRLDRNC

SEQ ID NO: 67 - protein, 7 aa, synthetic construct
LSLITRL

SEQ ID NO: 68 - protein, 41 aa, synthetic construct
NSLVLRGRMRDVKVRDDGRKSPSHHSKFSGGTRNWQKLVKL

SEQ ID NO: 69 - protein, 20 aa, synthetic construct
GLQRWCRWAESACGDSGPLR

SEQ ID NO: 70 - protein, 20 aa, synthetic construct
ATWQEYFRRGGSCSLAESCG

SEQ ID NO: 71 - protein, 20 aa, synthetic construct
CWRWIEYWSRGGQVGRRAGA

SEQ ID NO: 72 - protein, 40 aa, synthetic construct
SVVAWTGALRPSWVELRCLREVSDSGKMHPAWLDYCGELS

SEQ ID NO: 73 - RNA, 96 nt, Homo sapiens
  source 1..96: mol_type = genomic RNA
aaccattcaaatataccacagtttgtttaaccttttgcctgttggttgaagatgcctttcaacaggtgactggttagacaaactgtggtatataca

SEQ ID NO: 74 - RNA, 84 nt, Homo sapiens
  source 1..84: mol_type = genomic RNA
acgtcagggaaaggattctgctgtcggtcccactccaaagttcacagaatgggtggtgggcacagaatctggactctgcttgtg

SEQ ID NO: 75 - RNA, 22 nt, Homo sapiens
  source 1..22: mol_type = genomic RNA
tgggtctttgcgggcgagatga

SEQ ID NO: 76 - RNA, 73 nt, Homo sapiens
  source 1..73: mol_type = genomic RNA
cagggtgtgtgactggttgaccagaggggcatgcactgtgttcaccctgtgggccacctagtcaccaaccctc

SEQ ID NO: 77 - RNA, 124 nt, Homo sapiens
  source 1..124: mol_type = genomic RNA
aaccctccttgggaagtgaagctcaggctgtgatttcaagccagggggcgtttttctataactggatgaaaagcacctccagagcttgaagctcacagtttgagagcaatcgtctaaggaagtt

SEQ ID NO: 78 - RNA, 21 nt, Homo sapiens
  source 1..21: mol_type = genomic RNA
gcccctgggcctatcctagaa

SEQ ID NO: 79 - RNA, 129 nt, Homo sapiens
  source 1..129: mol_type = genomic RNA
gggatgccacattcagccattcagcgtacagtgcctttcacagggaggtgtcatttatgtgaactaaaatataaatttcacctttctgagaagggtaatgtacagcatgcactgcatatgtggtgtccc

SEQ ID NO: 80 - RNA, 22 nt, Homo sapiens
  source 1..22: mol_type = genomic RNA
aacaatatcctggtgctgagtg

SEQ ID NO: 81 - RNA, 22 nt, Homo sapiens
  source 1..22: mol_type = genomic RNA
cgcatcccctagggcattggtg

SEQ ID NO: 82 - RNA, 87 nt, Homo sapiens
  source 1..87: mol_type = genomic RNA
tgtcccccccggcccaggttctgtgatacactccgactcgggctctggagcagtcagtgcatgacagaacttgggcccggaaggacc

SEQ ID NO: 83 - RNA, 95 nt, Homo sapiens
  source 1..95: mol_type = genomic RNA
ccagctcgggcagccgtggccatcttactgggcagcattggatggagtcaggtctctaatactgcctggtaatgatgacggcggagccctgcacg

SEQ ID NO: 84 - RNA, 23 nt, Homo sapiens
  source 1..23: mol_type = genomic RNA
agctggtgttgtgaatcaggccg

SEQ ID NO: 85 - RNA, 87 nt, Homo sapiens
  source 1..87: mol_type = genomic RNA
agcttccctggctctagcagcacagaaatattggcacagggaagcgagtctgccaatattggctgtgctgctccaggcagggtggtg

SEQ ID NO: 86 - RNA, 89 nt, Homo sapiens
  source 1..89: mol_type = genomic RNA
gtcagcagtgccttagcagcacgtaaatattggcgttaagattctaaaattatctccagtattaactgtgctgctgaagtaaggttgac

SEQ ID NO: 87 - RNA, 83 nt, Homo sapiens
  source 1..83: mol_type = genomic RNA
ccttggagtaaagtagcagcacataatggtttgtggattttgaaaaggtgcaggccatattgtgctgcctcaaaaatacaagg

SEQ ID NO: 88 - RNA, 22 nt, Homo sapiens
  source 1..22: mol_type = genomic RNA
aactggcctacaaagtcccagt

SEQ ID NO: 89 - RNA, 110 nt, Homo sapiens
  source 1..110: mol_type = genomic RNA
ggccagctgtgagtgtttctttggcagtgtcttagctggttgttgtgagcaatagtaaggaagcaatcagcaagtatactgccctagaagtgctgcacgttgtggggccc

SEQ ID NO: 90 - RNA, 87 nt, Homo sapiens
  source 1..87: mol_type = genomic RNA
gacagtgcagtcacccataaagtagaaagcactactaacagcactggagggtgtagtgtttcctactttatggatgagtgtactgtg

SEQ ID NO: 91 - RNA, 98 nt, Homo sapiens
  source 1..98: mol_type = genomic RNA
cgaggggatacagcagcaattcatgttttgaagtgttctaaatggttcaaaacgtgaggcgctgctataccccctcgtggggaaggtagaaggtgggg

SEQ ID NO: 92 - RNA, 21 nt, Homo sapiens
  source 1..21: mol_type = genomic RNA
cagcagcacactgtggtttgt

SEQ ID NO: 93 - RNA, 100 nt, Homo sapiens
  source 1..100: mol_type = genomic RNA
tgtgtctctctctgtgtcctgccagtggttttaccctatggtaggttacgtcatgctgttctaccacagggtagaaccacggacaggataccggggcacc

SEQ ID NO: 94 - RNA, 95 nt, Homo sapiens
  source 1..95: mol_type = genomic RNA
ttgtacctggtgtgattataaagcaatgagactgattgtcatatgtcgtttgtgggatccgtctcagttactttatagccatacctggtatctta

SEQ ID NO: 95 - RNA, 73 nt, Homo sapiens
  source 1..73: mol_type = genomic RNA
ctcctcagatcagaaggtgattgtggctttgggtggatattaatcagccacagcactgcctggtcagaaagag

SEQ ID NO: 96 - RNA, 23 nt, Homo sapiens
  source 1..23: mol_type = genomic RNA
caaagtgcttacagtgcaggtag

SEQ ID NO: 97 - RNA, 65 nt, Homo sapiens
  source 1..65: mol_type = genomic RNA
ctgttaatgctaatcgtgataggggtttttgcctccaactgactcctacatattagcattaacag

SEQ ID NO: 98 - RNA, 83 nt, Homo sapiens
  source 1..83: mol_type = genomic RNA
cgccggccgatgggcgtcttaccagacatggttagacctggccctctgtctaatactgtctggtaaaaccgtccatccgctgc

SEQ ID NO: 99 - RNA, 87 nt, Homo sapiens
  source 1..87: mol_type = genomic RNA
caatagacacccatcgtgtcttttgctctgcagtcagtaaatatttttttgtgaatgtgtagcaaaagacagaatggtggtccattg

SEQ ID NO: 100 - RNA, 22 nt, Homo sapiens
  source 1..22: mol_type = genomic RNA
tagcaccatctgaaatcggtta

SEQ ID NO: 101 - RNA, 110 nt, Homo sapiens
  source 1..110: mol_type = genomic RNA
cggaaaatttgccaagggtttgggggaacattcaacctgtcggtgagtttgggcagctcaggcaaaccatcgaccgttgagtggaccctgaggcctggaattgccatcct

SEQ ID NO: 102 - RNA, 109 nt, Homo sapiens
  source 1..109: mol_type = genomic RNA
ggtcgggctcaccatgacacagtgtgagacctcgggctacaacacaggacccgggcgctgctctgacccctcgtgtcttgtgttgcagccggagggacgcaggtccgca

SEQ ID NO: 103 - DNA, 21 bp, synthetic construct
  source 1..21: mol_type = other DNA
gctccaaaggacttgtacgtg

SEQ ID NO: 104 - DNA, 21 bp, synthetic construct
  source 1..21: mol_type = other DNA
tgatctgaagggcagcatttc

SEQ ID NO: 105 - protein, 4 aa, synthetic construct
GFLG

SEQ ID NO: 106 - protein, 4 aa, synthetic construct
ALAL

SEQ ID NO: 107 - protein, 4 aa, synthetic construct
KAGG

SEQ ID NO: 108 - protein, 4 aa, synthetic construct
LRGG

SEQ ID NO: 109 - protein, 4 aa, synthetic construct
RKRR

SEQ ID NO: 110 - protein, 5 aa, synthetic construct
  VARIANT 5: note = Any amino acid
GLFGX

SEQ ID NO: 111 - protein, 5 aa, synthetic construct
  VARIANT 5: note = Any amino acid
GFLGX

SEQ ID NO: 112 - protein, 5 aa, synthetic construct
  VARIANT 5: note = Any amino acid
ALALX

SEQ ID NO: 113 - protein, 9 aa, synthetic construct
  SITE 4: note = Residue modified with a nitro group (NO2) located in the para position (4) of the benzene ring of the sidechain
  SITE 8: note = 4-hydroxymethylpyridine
  VARIANT 9: note = Any amino acid
FAAFFVLXX

SEQ ID NO: 114 - protein, 5 aa, synthetic construct
  SITE 1: note = Benzene modified residue
  SITE 5: note = 4-methoxy-beta-naphthylamine modified residue
RGFFP

SEQ ID NO: 115 - protein, 8 aa, synthetic construct
  VARIANT 8: note = Any amino acid
GGGRRRVX

SEQ ID NO: 116 - protein, 8 aa, synthetic construct
  SITE 1: note = Morpholinocarbonyl modified residue
  VARIANT 8: note = Any amino acid
HSSKLQLX

SEQ ID NO: 117 - protein, 5 aa, synthetic construct
  SITE 1: note = Acetylated residue
  VARIANT 5: note = Any amino acid
PLQLX

SEQ ID NO: 118 - protein, 9 aa, synthetic construct
  VARIANT 9: note = Any amino acid
GPLGIAGQX

SEQ ID NO: 119 - protein, 12 aa, synthetic construct
  SITE 1: note = Azidoacetic acid modified residue
YHWYGYTPQNVI

SEQ ID NO: 120 - DNA, 25 bp, synthetic construct
  source 1..25: mol_type = other DNA
  modified_base 25: mod_base = OTHER; note = Dibenzocyclooctyne modified nucleotide
  misc_feature 1..23: note = RNA
  misc_feature 24..25: note = DNA
ataggcattggtgaatttaaagaca

SEQ ID NO: 121 - RNA, 27 nt, synthetic construct
  source 1..27: mol_type = other RNA
tgtctttaaattcaccaatgcctatgc

SEQ ID NO: 122 - DNA, 22 bp, synthetic construct
  source 1..22: mol_type = other DNA
tccctcagccacccatatgtac

SEQ ID NO: 123 - DNA, 24 bp, synthetic construct
  source 1..24: mol_type = other DNA
gtctcgggccattttggagaatcc

SEQ ID NO: 124 - DNA, 21 bp, synthetic construct
  source 1..21: mol_type = other DNA
  misc_feature 1..19: note = RNA
  misc_feature 20..21: note = DNA
gactcaagatggaacctgatt

SEQ ID NO: 125 - DNA, 21 bp, synthetic construct
  source 1..21: mol_type = other DNA
  misc_feature 1..2: note = DNA
  misc_feature 3..21: note = RNA
ttctgagttctaccttggact

SEQ ID NO: 126 - protein, 12 aa, synthetic construct
NYSKPTDRQYHF

SEQ ID NO: 127 - DNA, 20 bp, synthetic construct
  source 1..20: mol_type = other DNA
tttttttttttttttttttt

(19) United States
(12) Patent Application Publication
(10) Pub. No.: US 2024/0325550 A1
(43) Pub. Date: Oct. 3, 2024

(54) Therapeutic Compounds for Inhibiting and Reducing the Expression of Cell Surface Proteins

(71) Applicants: KIST (Korea Institute of Science and Technology), Seongbuk-gu (KR); K2B Therapeutics, Inc., Cambridge, MA (US)

(72) Inventors: HoWon J. Kim, Lexington, MA (US); In-San Kim, Seoul (KR); Jay S. Kim, Bedford, MA (US); Sun Hwa Kim, Seoul (KR); Ick Chan Kwon, Seoul (KR); Jong Won Lee, Seoul (KR); Yoo Soo Yang, Seoul (KR); Hong Yeol Yoon, Gyeonggi-do (KR)

(21) Appl. No.: 18/618,258

(22) Filed: Mar. 27, 2024

Related U.S. Application Data
(63) Continuation of application No. 63/492,612, filed on Mar. 28, 2023.

(51) Int. Cl. A61K 47/64; A61P 35/00; C12N 15/113

(52) U.S. Cl. CPC A61K 47/64; A61P 35/00; C12N 15/113; C12N 2310/14; C12N 2310/141

(57) ABSTRACT

Therapeutic compounds for inhibiting and reducing the expression of cell surface proteins and methods for treating cancer, inflammation, and diabetes using the therapeutic compounds.

### CROSS-REFERENCE TO RELATED APPLICATIONS

This application claims priority from U.S. Provisional Application No. 63/492,612, filed Mar. 28, 2023, the disclosure of which is incorporated herein by reference in its entirety.

### SEQUENCE LISTING

The instant application contains a Sequence Listing which has been submitted electronically in XML file format and is hereby incorporated by reference in its entirety. Said XML copy, created on Mar. 15, 2024, is named 4813_1007_SL.xml and is 200,093 bytes in size.

### TECHNICAL FIELD

The present invention relates to compounds useful for treating cancer and diabetes, and more particularly to compounds configured to bind and inhibit a cell surface protein and, after internalization of the compound by a cell, reducing the expression of the cell surface protein.

### SUMMARY OF THE EMBODIMENTS

One embodiment of the invention provides a therapeutic compound comprising a conjugate configured to bind to a second protein expressed on a surface of a target cell, the conjugate comprising a first protein coupled to an API, wherein the conjugate is configured to bind to the second protein expressed on the surface of the target cell in a manner so as to inhibit an activity of the second protein, the conjugate is further configured to be internalized by the target cell upon binding to the second protein expressed on the surface of the target cell, the API is configured to be released from the conjugate after the conjugate has been internalized by the target cell, and the API is further configured to reduce the expression of the second protein expressed on the surface of the target cell after the API has been released from the conjugate, thereby further inhibiting the activity of the second protein, so that the conjugate and the API synergistically inhibit the activity of the second protein.

The API may be coupled to the first protein by a linker and the linker may be a cleavable linker.

In some embodiments, the second protein consists of a sequence selected from the group consisting of SEQ ID NOs: 18, 19, 20, 21, 22, 23, 24, 25, 26, 27, 29, 31, 32, 35, 36, 37, and 38. The API may be selected from the group consisting of a siRNA, an antisense oligonucleotide, and a microRNA.

In some embodiments, the second protein is PD-1 (SEQ ID NO:18) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 1, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is a microRNA, the microRNA consisting of a nucleic acid sequence, the nucleic acid sequence being at least 95% identical to SEQ ID NO: 39. In some embodiments, the microRNA consists of SEQ ID NO: 39. In still other embodiments, the API is an antisense oligonucleotide, the antisense oligonucleotide being 15-25 nucleotides in length and complementary to at least 15 contiguous nucleotides of SEQ ID NO:1. In some embodiments, the first protein is selected from the group consisting of an anti-PD-1 antibody and an antigen-binding fragment thereof. In other embodiments, the first protein is a PD-1 binding peptide consisting of an amino acid sequence, the amino acid sequence being at least 90% identical to a peptide sequence selected from the group consisting of SEQ ID NO:40 and SEQ ID NO:41. The PD-1 binding peptide may consist of an amino acid sequence selected from the group consisting of SEQ ID NO:40 and SEQ ID NO:41.

In some embodiments, the second protein is CD38 (SEQ ID NO:19) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 2, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is a microRNA, the microRNA consisting of a nucleic acid sequence, the nucleic acid sequence being at least 95% identical to a microRNA sequence selected from the group consisting of SEQ ID NO:42-44. In some embodiments, the microRNA consists of a nucleic acid sequence selected from the group consisting of SEQ ID NO:42-44. In other embodiments, the API is an antisense oligonucleotide, the antisense oligonucleotide being 15-25 nucleotides in length and complementary to at least 15 contiguous nucleotides of SEQ ID NO:2. In some embodiments, the first protein is selected from the group consisting of an anti-CD38 antibody and an antigen-binding fragment thereof. In other embodiments, the first protein is a CD38 binding peptide consisting of an amino acid sequence, the amino acid sequence being at least 90% identical to SEQ ID NO:45. The CD38 binding peptide may consist of SEQ ID NO:45.

In some embodiments, the second protein is HER2 (SEQ ID NO:20) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 3, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. The API may be a microRNA, the microRNA consisting of a nucleic acid sequence, the nucleic acid sequence being at least 95% identical to a microRNA sequence selected from the group consisting of SEQ ID NO:73-78. In some embodiments, the microRNA consists of a nucleic acid sequence selected from the group consisting of SEQ ID NO:73-78. In other embodiments, the API is an antisense oligonucleotide, the antisense oligonucleotide being 15-25 nucleotides in length and complementary to at least 15 contiguous nucleotides of SEQ ID NO:3. In some embodiments, the first protein is selected from the group consisting of an anti-HER2 antibody and an antigen-binding fragment thereof. In other embodiments, the first protein is a HER2 binding peptide consisting of an amino acid sequence, the amino acid sequence being at least 90% identical to a peptide sequence selected from the group consisting of SEQ ID NO:46-59. The HER2 binding peptide may consist of an amino acid sequence selected from the group consisting of SEQ ID NO:46-59.

In still other embodiments, the second protein is PD-L1 isoform A (SEQ ID NO:21) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 4, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is an antisense oligonucleotide, the antisense oligonucleotide being 15-25 nucleotides in length and complementary to at least 15 contiguous nucleotides of SEQ ID NO:4. In some embodiments, the first protein is selected from the group consisting of an anti-PD-L1 isoform A antibody and an antigen-binding fragment thereof.

In other embodiments, the second protein is PD-L1 isoform C (SEQ ID NO:22) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 5, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In some embodiments, the API is an antisense oligonucleotide, the antisense oligonucleotide being 15-25 nucleotides in length and complementary to at least 15 contiguous nucleotides of SEQ ID NO:5. In some embodiments, the first protein is selected from the group consisting of an anti-PD-L1 isoform C antibody and an antigen-binding fragment thereof.

The API may be a microRNA, the microRNA consisting of a nucleic acid sequence, the nucleic acid sequence being at least 95% identical to a microRNA sequence selected from the group consisting of SEQ ID NO:39, 79-96, and 97. In some embodiments, the microRNA consists of a nucleic acid sequence selected from the group consisting of SEQ ID NO: 39, 79-96, and 97. In other embodiments, the first protein is a PD-L1 binding peptide consisting of an amino acid sequence, the amino acid sequence being at least 90% identical to a peptide sequence selected from the group consisting of SEQ ID NO: 60-65. The PD-L1 binding peptide may consist of an amino acid sequence selected from the group consisting of SEQ ID NO:60-65.

One embodiment provides a method of treating cancer in a mammalian subject in need thereof, the method comprising administering a therapeutically effective amount of the therapeutic compound to the mammalian subject. The mammalian subject may be human.

In yet other embodiments, the second protein is IL4R isoform A (SEQ ID NO:23) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 6, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is an antisense oligonucleotide, the antisense oligonucleotide being 15-25 nucleotides in length and complementary to at least 15 contiguous nucleotides of SEQ ID NO:6. In some embodiments, the first protein is selected from the group consisting of an anti-IL4R isoform A antibody and an antigen-binding fragment thereof.

In some embodiments, the second protein is IL4R isoform C (SEQ ID NO:24) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 7, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is an antisense oligonucleotide, the antisense oligonucleotide being 15-25 nucleotides in length and complementary to at least 15 contiguous nucleotides of SEQ ID NO:7. In some embodiments, the first protein is selected from the group consisting of an anti-IL4R isoform C antibody and an antigen-binding fragment thereof.

The API may be a microRNA, the microRNA consisting of a nucleic acid sequence, the nucleic acid sequence being at least 95% identical to a microRNA sequence selected from the group consisting of SEQ ID NO:94 and 98. In some embodiments, the microRNA consists of a nucleic acid sequence selected from the group consisting of SEQ ID NO:94 and 98. In other embodiments, the first protein is a IL4R binding peptide consisting of an amino acid at least 90% identical to SEQ ID NO:66. The IL4R binding peptide may consist of SEQ ID NO:66.

In some embodiments, second protein is IL6R isoform 1 (SEQ ID NO:25) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 8, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is an antisense oligonucleotide, the antisense oligonucleotide being 15-25 nucleotides in length and complementary to at least 15 contiguous nucleotides of SEQ ID NO:8. In some embodiments, the first protein is selected from the group consisting of an anti-IL6R isoform 1 antibody and an antigen-binding fragment thereof.

In other embodiments, the second protein is IL6R isoform 2 (SEQ ID NO:26) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 9, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is an antisense oligonucleotide, the antisense oligonucleotide being 15-25 nucleotides in length and complementary to at least 15 contiguous nucleotides of SEQ ID NO:9. In some embodiments, the first protein is selected from the group consisting of an anti-IL6R isoform 2 antibody and an antigen-binding fragment thereof.

In still other embodiments, the second protein is IL6R isoform 3 (SEQ ID NO:27) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 10, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is an antisense oligonucleotide, the antisense oligonucleotide being 15-25 nucleotides in length and complementary to at least 15 contiguous nucleotides of SEQ ID NO:10. In some embodiments, the first protein is selected from the group consisting of an anti-IL6R isoform 3 antibody and an antigen-binding fragment thereof.

In some embodiments, the second protein is IL6R isoform 4 (SEQ ID NO:29) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 28, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is an antisense oligonucleotide, the antisense oligonucleotide being 15-25 nucleotides in length and complementary to at least 15 contiguous nucleotides of SEQ ID NO:28. In some embodiments, the first protein is selected from the group consisting of an anti-IL6R isoform 4 antibody and an antigen-binding fragment thereof.

In other embodiments, the second protein is IL6R isoform 5 (SEQ ID NO:31) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 30, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is an antisense oligonucleotide, the antisense oligonucleotide being 15-25 nucleotides in length and complementary to at least 15 contiguous nucleotides of SEQ ID NO:30. In some embodiments, the first protein is selected from the group consisting of an anti-IL6R isoform 5 antibody and an antigen-binding fragment thereof.

The first protein may be a IL6R binding peptide consisting of an amino acid at least 90% identical to SEQ ID NO:67. The IL6R binding peptide may consist of SEQ ID NO:67.

In some embodiments, the second protein is TNFR1 (SEQ ID NO:32) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 11, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is a microRNA, the microRNA consisting of a nucleic acid sequence, the nucleic acid sequence being at least 95% identical to a microRNA sequence selected from the group consisting of SEQ ID NO:99-102. In some embodiments, the microRNA consists of a nucleic acid sequence selected from the group consisting of SEQ ID NO:99-102. In still other embodiments, the API is an antisense oligonucleotide, the antisense oligonucleotide being 15-25 nucleotides in length and complementary to at least 15 contiguous nucleotides of SEQ ID NO:11. In some embodiments, the first protein is selected from the group consisting of an anti-TNFR1 antibody and an antigen-binding fragment thereof. In other embodiments, the first protein is a TNFR1 binding peptide consisting of an amino acid sequence, the amino acid sequence being at least 90% identical to SEQ ID NO:68. The TNFR1 binding peptide may consist of SEQ ID NO:68.

In some embodiments, the second protein is TNFR2 (SEQ ID NO:35) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 14, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is an antisense oligonucleotide, the antisense oligonucleotide being 15-25 nucleotides in length and complementary to at least 15 contiguous nucleotides of SEQ ID NO:14. In some embodiments, the first protein is selected from the group consisting of an anti-TNFR2 antibody and an antigen-binding fragment thereof. In other embodiments, the first protein is a TNFR1 binding peptide consisting of an amino acid sequence, the amino acid sequence being at least 90% identical to a peptide sequence selected from the group consisting of SEQ ID NO:69-72. The TNFR2 binding peptide may consist of an amino acid sequence selected from the group consisting of SEQ ID NO:69-72.

One embodiment provides a method of reducing inflammation in a mammalian subject in need thereof, the method comprising administering a therapeutically effective amount of the therapeutic compound to the mammalian subject. The mammalian subject may be human.

Another embodiment of the invention provides a therapeutic compound comprising a conjugate configured to bind to a sodium-dependent glucose cotransporter expressed on a surface of a target cell, the conjugate comprising a glucose coupled to an API, wherein the conjugate is configured to bind to the sodium-dependent glucose cotransporter so as to be transported across a membrane of the target cell and into the target cell, the API is configured to be released from the conjugate after the conjugate has been transported across the membrane of the target cell and into the target cell, and the API is further configured to reduce the expression of the sodium-dependent glucose cotransporter expressed on the surface of the target cell after the API has been released from the conjugate. The API may be coupled to the glucose by a linker and the linker may be a cleavable linker. In some embodiments, the API is selected from the group consisting of a siRNA and an antisense oligonucleotide.

In some embodiments, the sodium-dependent glucose cotransporter is SGLT1 isoform 1 (SEQ ID NO:36) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 15, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is an antisense oligonucleotide, the antisense oligonucleotide being 15-25 nucleotides in length and complementary to at least 15 contiguous nucleotides of SEQ ID NO:15.

In some embodiments, the sodium-dependent glucose cotransporter is SGLT1 isoform 2 (SEQ ID NO:37) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 16, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is an antisense oligonucleotide, the antisense oligonucleotide being 15-25 nucleotides in length and complementary to at least 15 contiguous nucleotides of SEQ ID NO:16.

In some embodiments, the sodium-dependent glucose cotransporter is SGLT2 (SEQ ID NO:38) and the API is an siRNA, the siRNA being a double-stranded RNA molecule including an antisense RNA strand and a sense RNA strand, wherein (a) the antisense RNA strand is 19-29 nucleotides in length and is complementary to contiguous nucleotides of SEQ ID NO: 17, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In other embodiments, the API is an antisense oligonucleotide, the antisense oligonucleotide being 15-25 nucleotides in length and complementary to at least 15 contiguous nucleotides of SEQ ID NO:17.

One embodiment provides a method of treating diabetes in a mammalian subject in need thereof, the method comprising administering a therapeutically effective amount of the therapeutic compound to the mammalian subject. The mammalian subject may be human.

In accordance with another embodiment, a pharmaceutical composition comprising the therapeutic compound and a pharmaceutically acceptable carrier.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing features of embodiments will be more readily understood by reference to the following detailed description, taken with reference to the accompanying drawings, in which:

FIG. 1 shows an SDS-PAGE gel and an agarose gel demonstrating the production of a PD-L1 binding peptide-siRNA conjugate of the correct size, in accordance with embodiments of the invention.

FIG. 2 shows microscopy images showing the binding of a Cy5.5-labeled PD-L1 binding peptide-siRNA conjugate to PD-L1 on the cell surface of CT26.CL25 cells, as demonstrated by significant Cy5.5 fluorescence on cells incubated with the Cy5.5-labeled PD-L1 binding peptide-siRNA conjugate (second from left), in accordance with embodiments of the invention. Untreated CT26.CL25 cells (left), CT26.CL25 cells pre-treated with an anti-PD-L1 antibody prior to incubation with the Cy5.5-labeled PD-L1 binding peptide-siRNA (second from right), and CT26.CL25 cells incubated only with the unconjugated Cy5.5-labeled siRNA (right), demonstrate little to no Cy5.5 fluorescence, indicating that the Cy5.5-labeled PD-L1 binding peptide-siRNA conjugate specifically binds PD-L1 on the cell surface via its PD-L1 binding peptide. Nuclei stained with Hoechst 33342 (blue), Cy5.5 fluorescence (red).

FIG. 3 shows microscopy images showing that a Cy5.5-labeled PD-L1 binding peptide-siRNA conjugate is internalized by CT26.CL25 cells to a significantly greater degree than the unconjugated Cy5.5-labeled siRNA at 9 hours, 18 hours, and 24 hours after treatment with the Cy5.5-labeled PD-L1 binding peptide-siRNA or the unconjugated Cy5.5-labeled siRNA, in accordance with embodiments of the invention.

FIG. 4 shows microscopy images showing that a Cy5.5-labeled PD-L1 binding peptide-siRNA conjugate is internalized by B16F10 cells to a significantly greater degree than the unconjugated Cy5.5-labeled siRNA at 18 hours and 24 hours after treatment with the Cy5.5-labeled PD-L1 binding peptide-siRNA or the unconjugated Cy5.5-labeled siRNA, in accordance with embodiments of the invention. Nuclei stained with Hoechst 33342 (blue), Cy5.5 fluorescence (red).

FIG. 5 shows a bar chart of qRT-PCR results demonstrating a significant reduction in relative PD-L1 expression in Cy5.5-labeled PD-L1 binding peptide-siRNA conjugate-treated CT26.CL25 cells versus untreated CT26.CL25 cells, in accordance with embodiments of the invention.

FIG. 6 shows a bar chart demonstrating that pre-treatment of 4T1 cells with Cy5.5-labeled PD-L1 binding peptide-siRNA conjugate reduces the subsequent binding of an APC-labeled anti-PD-L1 antibody to a greater degree than pre-treatment of 4T1 cells with an unconjugated PD-L1 binding protein, in accordance with embodiments of the invention.

FIG. 7 shows a bar chart of Western blot results demonstrating a significant reduction in relative PD-L1 protein expression in Cy5.5-labeled PD-L1 binding peptide-siRNA conjugate-treated CT26.CL25 cells versus siRNA conjugate-only treated and untreated CT26.CL25 cells, in accordance with embodiments of the invention.

FIG. 8 shows microscopy images showing the binding of an EGFR binding peptide-siRNA conjugate to EGFR on the cell surface of A549 cells, as demonstrated by significant YOYO-1 staining on cells incubated with the YOYO-1-stained EGFR binding peptide-siRNA conjugate, in accordance with embodiments of the invention. Untreated cells and cells treated with unconjugated YOYO-1-stained EGFR siRNA display no YOYO-1 staining.

FIG. 9 shows a bar chart of qRT-PCR results demonstrating a significant reduction in relative EGFR expression in EGFR binding peptide-siRNA conjugate-treated A549 cells versus untreated cells and cells treated with unconjugated EGFR siRNA, in accordance with embodiments of the invention.

FIG. 10 shows a bar chart of Western blot results demonstrating a significant reduction in relative EGFR expression in EGFR binding peptide-siRNA conjugate-treated A549 cells versus untreated cells and cells treated with unconjugated EGFR siRNA, in accordance with embodiments of the invention.

### DETAILED DESCRIPTION OF SPECIFIC EMBODIMENTS

Definitions. As used in this description and the accompanying claims, the following terms shall have the meanings indicated, unless the context otherwise requires.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

A “set” includes at least one member.

The therapeutic compounds described herein are conjugates comprising a “warhead” coupled to an active pharmaceutical ingredient (“API”). The warhead may be a protein or a small molecule that binds to a particular protein expressed on the surface of a cell. For example, PD-1 binding peptide (SEQ ID NO:40) may be the warhead in a conjugate comprising PD-1 binding peptide (SEQ ID NO:40) coupled to an API. PD-1 binding peptide (SEQ ID NO:40) binds PD-1 expressed on the surface of a cell. Similarly, glucose is the warhead in a conjugate comprising glucose coupled to an API. Glucose binds to sodium-dependent glucose cotransporters expressed on the surface of a cell.

A “target cell” is a cell expressing a protein on its cell surface that a therapeutic compound binds, the expression of the protein on the cell surface of the target cell being reduced after said binding and subsequent internalization of the therapeutic compound by the target cell.

“Active pharmaceutical ingredient,” “API,” and the like, means the non-warhead portion and the non-linker portion of a therapeutic compound that is biologically active, in accordance with embodiments of the invention. Suitable active pharmaceutical ingredients (“APIs”) include nucleic acid molecules such as siRNA, miRNA, antisense oligonucleotides, and derivatives thereof, including, but not limited to, nucleic acid molecules comprising a modified nucleotide, backbone, sugar, and/or base.

“Complementarity,” as used herein regarding nucleic acid sequences, refers to the ability of a nucleic acid to forms hydrogen bonds with another nucleic acid sequence by Watson-Crick base pairing or wobble base pairing. A percent complementarity indicates the percentage of nucleotides in a nucleic acid molecule that can form hydrogen bonds (e.g., Watson-Crick base pairing) with the nucleotides of a second nucleic acid sequence (e.g., 5, 6, 7, 8, 9, 10 out of 10 being 50%, 60%, 70%, 80%, 90%, and 100% complementarity). “Perfectly complementary,” and the like, means that all the contiguous nucleotides of a nucleic acid sequence will hydrogen bond with the same number of contiguous nucleotides in a second nucleic acid sequence (i.e., the nucleic acid sequence has 100% complementarity). “Complementary,” as used herein without further qualification, means that contiguous nucleotides of a nucleic acid sequence has a percent complementarity with contiguous nucleotides of a second nucleic acid sequence that is selected from the group consisting of 92%, 93%, 94%, 95%, 96%, 97%, 98%, 99%, and 100%. For example, a nucleic acid sequence that is 19 nucleotides in length and complementary to 14 nucleotides of a second nucleic acid sequence means that 14 contiguous nucleotides of the nucleic acid sequence has a percent complementarity with contiguous nucleotides of the second nucleic acid sequence that is selected from the group consisting of 92%, 93%, 94%, 95%, 96%, 97%, 98%, 99%, and 100% complementarity. A nucleic acid sequence that is 19 nucleotides in length and complementary to at least 15 contiguous nucleotides in a second nucleic acid sequence means that contiguous nucleotides of the nucleic acid sequence has a percent complementarity with at least 15 contiguous nucleotides of the second nucleic acid sequence that is selected from the group consisting of 92%, 93%, 94%, 95%, 96%, 97%, 98%, 99%, and 100% complementarity.

The term “antibody” refers to an immunoglobulin molecule that is typically composed of two identical pairs of polypeptide chains, each pair having one “heavy” (H) chain and one “light” (L) chain. Human light chains are classified as kappa (κ) and lambda (λ). Heavy chains are classified as mu, delta, gamma, alpha, or epsilon, and define the antibody's isotype as IgM, IgD, IgG, IgA, and IgE, respectively. Each heavy chain is comprised of a heavy chain variable region (VH) and a heavy chain constant region. The heavy chain constant regions of IgD, IgG, and IgA are comprised of three domains, CH1, CH2 and CH3, and the heavy chain constant regions of IgM and IgE are comprised of four domains, CH1, CH2, CH3, and CH4. Each light chain is comprised of a light chain variable region (VL) and a light chain constant region. The light chain constant region is comprised of one domain, CL. The constant regions of the antibodies may mediate the binding of the immunoglobulin to host tissues or factors, including various cells of the immune system (e.g., effector cells). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from the amino-terminus to carboxy-terminus in the following order: FR1, CDR1, FR2, CDR2, FR3, CDR3, FR4. The variable regions of each heavy/light chain pair (VH/VL) typically form an antibody's antigen-binding site. The term “antibody” is not limited by any particular method of producing the antibody. For example, it includes monoclonal antibodies, recombinant antibodies, and polyclonal antibodies.

The term “human antibody” refers to an antibody consisting of amino acid sequences of human immunoglobulin sequences only. A human antibody may contain murine carbohydrate chains if produced in a mouse, in a mouse cell or in a hybridoma derived from a mouse cell. Human antibodies may be prepared in a variety of ways known in the art.

The term “humanized antibody” refers to an antibody that contains some or all of the CDRs from a non-human animal antibody, while the framework and constant regions of the antibody contain amino acid residues derived from human antibody sequences. Humanized antibodies are typically produced by grafting CDRs from a mouse antibody into human framework sequences followed by back substitution of certain human framework residues for the corresponding mouse residues from the source antibody. The term “humanized antibody” also refers to an antibody of non-human origin in which, typically in one or more variable regions, one or more epitopes have been removed that have a high propensity of constituting a human T-cell and/or B-cell epitope, for purposes of reducing immunogenicity. The amino acid sequence of the epitope can be removed in full or in part. However, typically the amino acid sequence is altered by substituting one or more of the amino acids constituting the epitope for one or more other amino acids, thereby changing the amino acid sequence into a sequence that does not constitute a human T-cell and/or B-cell epitope. The amino acids are substituted by amino acids that are present at the corresponding position(s) in a corresponding human variable heavy or variable light chain as the case may be.

An “antigen-binding fragment” of an antibody refers to a fragment of an antibody that binds to an antigen, comprising a constant and a variable domain of each of the heavy and the light chain of an antibody. Examples of antigen-binding fragments include Fab fragments and F(ab′)₂ fragments.

“Therapeutically effective amount” means an amount of a therapeutic compound or composition sufficient to provide a desired biological result. That result can be reduction and/or alleviation of the signs, symptoms, or causes of a disease, or any other desired alteration of a biological system. An appropriate “therapeutically effective amount” in any individual case may be determined by one of ordinary skill in the art using routine experimentation.

The term “pharmaceutically acceptable carrier” means solvents, carrier agents, diluting agents and the like which are usually used in the administration of pharmaceutical compounds.

The term “identical” as used in reference to two sequences means that the monomeric units of one of the sequences matches exactly the monomeric units of the other sequence when the two sequences are aligned. Two sequences that are exactly the same share 100% identity and are referred to as being 100% identical. The monomeric unit of a protein/peptide sequence is an amino acid residue and the monomeric unit of a nucleic acid sequence is a nucleotide. Similarly, a sequence that is 90% identical to a second sequence means that 90% of the monomeric units of the sequence exactly match the monomeric units of the second sequence when the two sequences are aligned. For example, a protein having a first amino acid sequence that is 90% identical to second amino acid sequence of 20 amino acid residues means that, when the first sequence is aligned with the second sequence, the first sequence differs from the second sequence by two amino acid residues. Sequence identity and sequence alignment are well-known to those of skill in the art.

### One-Two Punch

Certain cell surface proteins are known to play a role in the proliferation of cancers of various forms. These cell surface proteins include, e.g., programmed cell death protein 1 (“PD-1”), programmed cell death ligand 1 (“PD-L1”), cluster of differentiation 38 (“CD38”), and human epidermal growth factor receptor 2 (“HER2”). By binding and inhibiting one or more of these proteins on the surface of a cancer cell, cancer cell proliferation can be suppressed or eliminated.

Other cell surface proteins, such as interleukin 4 receptor (“IL4R”), interleukin 6 receptor (“IL6R”), tumor necrosis factor receptor 1 (“TNFR1”), and tumor necrosis factor receptor 2 (“TNFR2”) play a role in inflammation. By binding and inhibiting one or more of IL4R, IL6R, TNFR1, and TNFR2, inflammation in a subject can be reduced or eliminated.

Finally, cell surface proteins such as sodium-dependent glucose cotransporter protein 1 (“SGLT-1”) and sodium-dependent glucose cotransporter protein 2 (“SGLT-2”), offer promising targets for treating diabetes. By binding and inhibiting one or more of SGLT-1 and SGLT-2, sugar uptake by the intestine (i.e., cells expressing SGLT-1) or the tubular system of the kidney (i.e., cells expressing SGLT-2), which can be useful in treating diabetes in a subject.

Here, we describe novel therapeutic compounds useful for treating various conditions, such as cancer, inflammation, and diabetes, comprising a conjugate having a warhead (for example, a peptide configured to bind a cell surface protein on the surface of a cancer cell) coupled to an API that are configured to: (1) bind a cell surface protein expressed on the surface of a cell, thereby inhibiting the activity of the cell surface protein, and (2) be internalized by the cancer cell, thereby delivering the API into the cancer cell, the API being configured to be released from the conjugate after internalization by the cell and to reduce expression of the cell surface protein. We have termed the dual effect of such conjugates on inhibiting activity of a cell surface protein upon binding the cell surface protein, and reducing the expression of the cell surface protein after internalization of the conjugate, a “one-two punch.” In some embodiments, therapeutic compounds disclosed herein may be administered to a subject so as to produce a one-two punch effect, thereby inhibiting the activity of a cell surface protein selected from the group consisting of PD-1, PD-L1, CD38, HER2, IL4R, IL6R, TNFR1, and TNFR2.

Table 1 provides “warhead” peptides that bind the indicated cell surface protein, in accordance with an embodiment of the invention. These warhead peptides may be coupled to an API so as to form a conjugate configured to deliver a one-two punch to a target cell.

TABLE 1
Warhead Peptides and the Cell Surface Proteins they Bind

| Warhead Peptide | Cell Surface Protein |
|---|---|
| PD-1 Binding Peptide | PD-1 (SEQ ID NO: 18) |
| (SEQ ID NO: 40 and 41) | |
| CD38 Binding Peptide | CD38 (SEQ ID NO: 19) |
| (SEQ ID NO: 45) | |
| HER 2 Binding Peptide | HER2 isoform A (SEQ ID NO: 20) |
| (SEQ ID NO: 46-59) | |
| PD-L1 Binding Peptide | PD-L1 isoform A (SEQ ID NO: 21) |
| (SEQ ID NO: 60-65) | PD-L1 isoform C (SEQ ID NO: 22) |
| IL4R Binding Peptide | IL4R isoform A (SEQ ID NO: 23) |
| (SEQ ID NO: 66) | IL4R isoform C (SEQ ID NO: 24) |
| IL6R Binding Peptide | IL6R isoform 1 (SEQ ID NO: 25) |
| (SEQ ID NO: 67) | IL6R isoform 2 (SEQ ID NO: 26) |
| | IL6R isoform 3 (SEQ ID NO: 27) |
| | IL6R isoform 4 (SEQ ID NO: 29) |
| | IL6R isoform 5 (SEQ ID NO: 31) |
| TNFR1 Binding Peptide | TNFR1 (SEQ ID NO: 32) |
| (SEQ ID NO: 68) | |
| TNFR2 Binding Peptide | TNFR2 (SEQ ID NO: 35) |
| (SEQ ID NO: 69-72) | |

### PD-1

Programmed cell death protein 1 (PD-1) is a type of immune checkpoint protein that regulates the activity of the immune system. It is expressed on the surface of immune cells, including T cells and B cells, and plays a crucial role in maintaining immune tolerance by inhibiting the activation and proliferation of immune cells.

However, PD-1 has also been found to be upregulated in cancer cells, leading to immune evasion and decreased immune response to the cancer cells. This allows the cancer cells to evade detection and attack by the immune system, leading to the growth and progression of the cancer. Studies have shown that targeting PD-1 with monoclonal antibodies, such as pembrolizumab and nivolumab, can enhance the immune response to cancer cells and improve outcomes in certain cancer types, including melanoma, non-small cell lung cancer, and renal cell carcinoma. Chen, L., & Mellman, I. (2014). Nature, 541 (7637), 321-330 and Balar, A V & Weber, J S. (2017) PD-1 and PD-L1 antibodies in cancer: current status and future directions, Cancer Immunol Immunother., 66(5): 551-564.

### CD38

CD38 is a transmembrane protein that is expressed on the surface of various immune cells, including T cells, B cells, and myeloid cells, as well as on certain cancer cells. It is involved in various immune and signaling pathways, including the regulation of calcium levels, the activation of enzymes, and the modulation of immune responses.

CD38 has been identified as a potential therapeutic target in cancer, as it is often overexpressed on the surface of cancer cells, particularly in lymphoma, multiple myeloma, and acute myeloid leukemia. Overexpression of CD38 on cancer cells has been associated with a poorer prognosis and resistance to chemotherapy. In addition to its expression on cancer cells, CD38 is also involved in the immune response to cancer. CD38-expressing immune cells, such as T cells, play a role in the recognition and destruction of cancer cells. Several studies have demonstrated the potential of targeting CD38 in cancer therapy. For example, a monoclonal antibody targeting CD38 has shown promising results in the treatment of multiple myeloma, and small molecule inhibitors of CD38 have shown potential in the treatment of lymphoma and leukemia. Wang, X & Li, G. (2016). CD38: A multifunctional protein in cancer and immunity. Frontiers in Oncology, 6, 114 and Chen, W. & Chen, J. (2017). CD38: A potential target for cancer immunotherapy. Cancer Letters, 390, 191-199.

### HER2

HER2, also known as human epidermal growth factor receptor 2, is a protein that is expressed on the surface of cells in the human body. It is a member of the epidermal growth factor receptor (EGFR) family, which is involved in the regulation of cell proliferation and survival. The terms “HER2” and “EGFR” are used interchangeably and synonymously herein, e.g., an EGFR-binding peptide is a HER2-binding peptide.

HER2 is often overexpressed in certain types of cancer, including breast, ovarian, gastric, and pancreatic cancer. Overexpression of HER2 leads to an uncontrolled proliferation of cancer cells and can result in the development of aggressive and therapy-resistant tumors. The overexpression of HER2 in cancer cells has been identified as a key driver of tumor progression and is associated with a poor prognosis. In breast cancer, for example, HER2 overexpression is found in approximately 20% of cases and is associated with a more aggressive form of the disease and a higher risk of relapse.

Therapies targeting HER2 have been developed and have shown to be effective in the treatment of HER2-positive cancers. These therapies include monoclonal antibodies such as trastuzumab and pertuzumab, which bind to HER2 and inhibit its signaling, and small molecule tyrosine kinase inhibitors such as lapatinib and neratinib, which block the activity of HER2. Tan, M., Yu, D. (2007). “Molecular mechanisms of erbB2-mediated breast cancer chemoresistance”. Breast Cancer Chemosensitivity. Advances in Experimental Medicine and Biology. Vol. 608. pp. 119-29 and Vranid, S. et al. (2021). “Targeting HER2 expression in cancer: New drugs and new indications”. Bosnian Journal of Basic Medical Sciences. 21 (1): 1-4.

### PD-L1

Programmed death-ligand 1(PD-L1) is a protein that is expressed on the surface of certain cells, including cancer cells. It belongs to the immune checkpoint family of proteins, which regulate the activity of the immune system. PD-L1 is able to bind to a receptor called PD-1, which is expressed on the surface of immune cells called T cells. When PD-L1 and PD-1 bind together, they inhibit the activity of T cells, which can help to prevent the immune system from attacking normal cells. However, cancer cells can exploit this inhibitory pathway by expressing high levels of PD-L1, which helps them evade the immune system and continue to grow and spread.

In recent years, PD-L1 has emerged as a target for cancer immunotherapy. PD-L1 inhibitors are a class of drugs that block the interaction between PD-L1 and PD-1, allowing the immune system to attack cancer cells. These drugs have shown promising results in a number of cancer types, including lung, bladder, and kidney cancer. Balar, A V & Weber, J S. (2017) PD-1 and PD-L1 antibodies in cancer: current status and future directions, Cancer Immunol Immunother., 66(5): 551-564.

### IL-4R

Interleukin-4 receptor (IL-4R) is a transmembrane protein that plays a key role in the immune system. It is expressed on various immune cells, including T-helper cells, B cells, and macrophages, and is activated by the cytokine interleukin-4 (IL-4). IL-4 is a key cytokine involved in the immune response to infections and inflammation. It is primarily produced by T-helper 2(Th2) cells and promotes the differentiation of immune cells into Th2 cells, which are involved in the production of antibodies and the activation of eosinophils and mast cells.

IL-4R activation leads to downstream signaling pathways that regulate immune cell activation and function. It has been shown to promote the production of anti-inflammatory cytokines, such as IL-10, and inhibit the production of pro-inflammatory cytokines, such as IL-1 and TNF-alpha. In addition, IL-4R activation has been shown to play a role in the development and maintenance of allergic inflammation resulting in atopic dermatitis and asthma. Allergic inflammation is characterized by the activation of Th2 cells and the production of IL-4 and IL-13, which lead to the activation of eosinophils and the release of histamine from mast cells. Nelms, K. et al. (1999) Annu. Rev. Immunol., 17: 701-38.

### IL-6R

The IL-6 receptor (IL-6R) is a protein that is expressed on the surface of cells and is responsible for binding and activating Interleukin 6 (IL-6). When IL-6 binds to IL-6R, it activates signaling pathways within the cell that lead to the production of various proteins, including those involved in inflammation. IL-6 is a cytokine that plays a key role in the immune system, particularly in the inflammatory response. It is produced by various immune cells, including T cells, B cells, and monocytes, and is involved in a wide range of immune processes, including inflammation, hematopoiesis, and immune cell activation.

One of the main functions of IL-6 in inflammation is the activation of T cells and B cells. It also activates neutrophils and monocytes, which are important I the initial stages of inflammation, as well as macrophages, which play a key role in the resolution of inflammation. Kaur, S. et al. (2020) Bioorg Med Chem 28(5):115327.

### TNFR1/TNFR2

Two receptors have been identified to mediate interactions with TNF, tumor necrosis factor receptor 1 (TNFR1), also called CD120a and p55 (its molecular weight is 55 kDa), and tumor necrosis factor receptor 2 (TNFR2), also called CD120b and p75 (its molecular weight is 75 kDa) [29]. TNFR1 and TNFR2 are not specific to TNF, they interact also with lymphotoxin alpha (LTα, previously known as TNFβ). LTα is a cytokine closely related to TNF, activated by similar stimuli than those activating TNF, produced mainly by lymphoid cells in a soluble form and can combine with LTβ interacting with another different receptor, LTβR [30].

TNFR1 and TNFR2 are on the cellular membrane or in a soluble form following TACE activation; their cytoplasmic domains are unrelated, and intracellular signaling pathways are independent. TNFR1 is involved in cytotoxicity, whereas TNFR2 plays a role in cytotoxicity and proliferation [31,32]. The exact mechanism involving TACE in the shedding of TNFR1 and TNFR2 is still unclear.

TNFR1 and TNFR2 are single-spanning type I transmembrane proteins characterized by having several cysteine-rich domains (CRDs) in their extracellular domain. Soluble forms of TNFR1 and TNFR2 have also been described and result from alternative splicing or shedding. The soluble TNF receptor variants inhibit TNF by competing with the cellular receptor species for TNF binding but possibly also by acting as dominant-negative molecules. Indeed, the N-terminal CRDs of TNFR1 and TNFR2 are not directly involved in ligand binding but mediate inactive self-association in the absence of ligand. This part of the TNF receptors has therefore been named pre-ligand binding assembly domain (PLAD) and seems to be a prerequisite for ligand binding and subsequent formation of active receptor complexes. Thus, soluble TNF receptor molecules might also act as TNF inhibitors by formation of inactive complexes with cellular TNF receptors by PLAD-PLAD interaction, but this issue has not been clarified yet. Front. Cell Dev. Biol., 29 May 2019 (doi.org/10.3389/fcell.2019.00091) and Int J Mol Sci. 2021 June; 22(11): 5461. (doi: 10.3390/ijms22115461).

In addition, we describe novel therapeutic compounds useful for treating diabetes comprising a conjugate having glucose as a warhead coupled to an API. The conjugate is configured to bind to a sodium-dependent glucose cotransporter expressed on the surface of a cell so as to be transported across the membrane of the target cell and into the target cell. Once transported across the membrane, the API reduces the expression of a sodium-dependent glucose cotransporter.

### SGLT1/SGLT2

Sodium-dependent glucose cotransporters include sodium-glucose cotransporter 1 (“SGLT1”) and sodium-glucose cotransporter 2 (“SGLT2”). SGLT1 and SGLT2 are expressed on the surface of certain cells in the small intestine and in the proximal tubule of the nephron.

Glucose can bind to, and be transported across a cell membrane by, a sodium-dependent glucose cotransporter expressed on the surface of a cell, the sodium-dependent glucose cotransporter being selected from the group consisting of SGLT1 isoform 1 (SEQ ID NO:36), SGLT1 isoform 2 (SEQ ID NO:37), and SGLT2 (SEQ ID NO:38). Glucose may be utilized as a “warhead” when coupled to an API so as to form a conjugate configured to be transported across the membrane of a cell expressing a sodium-dependent glucose cotransporter selected from the group consisting of SGLT1 isoform 1 (SEQ ID NO:36), SGLT1 isoform 2 (SEQ ID NO:37), and SGLT2 (SEQ ID NO:38).

### Linkers

In some embodiments, the therapeutic compounds described herein include a warhead coupled to an API by a linker. Suitable linkers include, but are not limited to, cleavable linkers such as hydrazone linkers, imine linkers, oxime linkers, carbonate linkers, acetal linkers, orthoester linkers, silyl ether linkers, disulfide linkers, trioxolane linkers, beta-glucuronide linkers, beta-galactoside linkers, pyrophosphate linkers, phosphoramidate linkers, arylsulfate linkers, heptamethine cyanine linkers, nitrobenzyl linkers, aryl boronic acid linkers, boronate linkers, thioether linkers, maleimidocaproyl-containing linkers, enzyme-cleavable peptide linkers, and para-amino benzyl carbamate-containing linkers, as well as non-cleavable linkers such as polyethylene glycol.

The conjugates described herein may be made by via linker using coupling reactions such as bis(vinylsulfonyl)piperazine-disulfide coupling, N-methyl-N-phenylvinylsulfonamide-cysteine coupling, platinum (II) compound-histidine coupling, and tetrazine-trans-cyclooctene coupling. Suitable linkers and coupling reactions are known to those of skill in art. See, e.g., Su et al. Acta Pharmaceutica Sinica B (2021), ISSN 2211-3835; Pan et al. Med Res Rev. 40:2682-2713 (2020); Khongorzul et al. Mol Cancer Res 18:3-19 (2020); Bargh et al. Chem Soc Rev 48:4361-4374 (2019); and Smith et al. Pharm Res 32:3526-3540 (2015), each of which is hereby incorporated by reference herein in its entirety.

Various enzyme-cleavable peptide linkers, such as those described below, can be used to make the conjugates described herein, in accordance with embodiments of the invention. These linkers comprise amino acid residues and are cleaved by specific enzymes within a cell, such as lysosomal degradative enzymes. See, e.g., Kong et al. J Biol Chem 290:7160-7168 (2015); Poreba FEBS J 287:1936-1969 (2020); and Singh et al., Current Medicinal Chemistry 15(18) (2008), each of which is hereby incorporated by reference herein in its entirety. Exemplary peptide linkers suitable for use in accordance with embodiments of the invention are described below.

For example, di-peptide linkers are comprised of two amino acid residues that serve as a recognition motif for cleavage by the enzyme cathepsin B, which cleaves the amide bond after the second amino acid residue between the carbonyl and amine. Di-peptide linkers cleaved by cathepsin B include Phe-Arg, Phe-Cit, Phe-Lys, Ala-Arg, Ala-Cit, Val-Ala, Val-Arg, Val-Lys, Val-Cit, and Arg-Arg. Cathepsin B similarly recognizes and cleaves the tetra-peptide linkers Gly-Phe-Leu-Gly (SEQ ID NO: 105) and Ala-Leu-Ala-Leu (SEQ ID NO: 106) after the fourth amino acid residue.

In addition, the tri-peptide linker Ala-Ala-Asn is cleaved by the enzyme legumain after the last amino acid residue. The tetra-peptide linkers Lys-Ala-Gly-Gly (SEQ ID NO: 107), Leu-Arg-Gly-Gly (SEQ ID NO: 108), and Arg-Lys-Arg-Arg (SEQ ID NO: 109) are cleaved by the papain-like protease enzyme.

Peptide linkers Arg-Arg-X, Ala-Leu-X, Gly-Leu-Phe-Gly-X (SEQ ID NO: 110), Gly-Phe-Leu-Gly-X (SEQ ID NO: 111), and Ala-Leu-Ala-Leu-X (SEQ ID NO: 112), where X is any amino acid, are cleaved by the enzymes cathepsin B, H, and L. Cathepsin B, H, and L are responsible for lysosomal degradation of proteins.

Peptide linkers Phe-Ala-Ala-Phe(NO₂)-Phe-Val-Leu-OM4P-X (SEQ ID NO: 113) and Bz-Arg-Gly-Phe-Phe-Pro-4moNA (SEQ ID NO: 114), where X is any amino acid, are cleaved by the enzyme cathepsin D.

Serum plasminogen activator is produced in many tumor cells. Plasminogen is converted to plasmin, thus producing a high-level of plasmin in the tumor cells. This plasmin is degraded rapidly in the plasma and hence tissues remote to the tumor are not exposed to plasmin. Plasmin is responsible for the fibrinolysis and degradation of blood plasma proteins and cleaves the peptide linkers D-Val-Leu-Lys-X, D-Ala-Phe-Lys-X, and D-Ala-Trp-Lys-X, where X is any amino acid.

Tissue plasminogen activator (tPA) and urokinase (uPA) are responsible for activation of plasmin formation and can each cleave the peptide linker Gly-Gly-Gly-Arg-Arg-Arg-Val-X (SEQ ID NO: 115), where X is any amino acid.

Prostate-specific antigen is responsible for liquefaction of semen and cleaves the peptide linker morpholinocarbonyl-His-Ser-Ser-Lys-Leu-Gln-Leu-X (SEQ ID NO: 116), where X is any amino acid.

Matrix metalloproteases (MMP-2 and MM-9) are responsible for degradation of extracellular matrix and collagens and cleave the peptide linkers Ac-Pro-Leu-Gln-Leu-X (SEQ ID NO: 117) and Gly-Pro-Leu-Gly-Ile-Ala-Gly-Gln-X (SEQ ID NO: 118), where X is any amino acid.

### APIs and Therapeutic Compounds Coupled Thereto

siRNA

In accordance with some embodiments, an API may be small-interfering RNA (“siRNA”). siRNA is a double-stranded RNA molecule that can reduce the expression of a specific gene by causing the degradation of the gene's mRNA transcript(s), which shares partial complementarity with a strand of the double-stranded siRNA molecule. The process of reducing the expression of a gene using siRNA is referred to as RNA interference (“RNAi”). See U.S. Pat. Nos. 7,056,704, 7,078,196, 8,372,968 each of which is hereby incorporated by reference herein its entirety.

The transcripts encoding the cell surface proteins shown in Table 2 are targets for the use of siRNA as an API to reduce expression of the indicated cell surface protein via RNAi.

TABLE 2
Cell Surface Protein Transcripts

| Cell Surface Protein Transcript | Cell Surface Protein |
|---|---|
| PD-1 transcript (SEQ ID NO: 1) | PD-1 |
| CD38 transcript (SEQ ID NO: 2) | CD38 |
| HER2 transcript variant 1 (SEQ ID NO: 3) | HER2 isoform A |
| PD-L1 transcript variant 1(SEQ ID NO: 4) | PD-L1 isoform A |
| PD-L1 transcript variant 4 (SEQ ID NO: 5) | PD-L1 isoform C |
| IL4R transcript variant 1 (SEQ ID NO: 6) | IL4R isoform A |
| IL4R transcript variant 4 (SEQ ID NO: 7) | IL4R isoform C |
| IL6R transcript variant 1 (SEQ ID NO: 8) | IL6R isoform 1 |
| IL6R transcript variant 2 (SEQ ID NO: 9) | IL6R isoform 2 |
| IL6R transcript variant 3 (SEQ ID NO: 10) | IL6R isoform 3 |
| IL6R transcript variant 4 (SEQ ID NO: 28) | IL6R isoform 4 |
| IL6R transcript variant 5 (SEQ ID NO: 30) | IL6R isoform 5 |
| TNFR1 transcript (SEQ ID NO: 11) | TNFR1 |
| TNFR2 transcript (SEQ ID NO: 14) | TNFR2 |
| SGLT1 transcript variant 1 (SEQ ID NO: 15) | SGLT1 isoform 1 |
| SGLT1 transcript variant 2 (SEQ ID NO: 16) | SGLT1 isoform 2 |
| SGLT2 transcript (SEQ ID NO: 17) | SGLT2 |

In accordance with some embodiments, a therapeutic compound of the invention comprises a first protein, the first protein being selected from the group consisting of (i) a PD-1 binding peptide consisting of an amino acid sequence that is at least 90% identical to a sequence selected from the group consisting of SEQ ID NO:40 and SEQ ID NO:41 and (ii) an anti-PD-1 antibody or antigen binding fragment thereof, coupled to an API, the API being an siRNA, the siRNA being a double-stranded RNA molecule including an antisense RNA strand and a sense RNA strand, wherein: (a) the antisense RNA strand is 19-29 nucleotides in length and is complementary to contiguous nucleotides of SEQ ID NO: 1, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In some embodiments, the PD-1 binding peptide consists of an amino acid sequence selected from the group consisting of SEQ ID NO:40 and SEQ ID NO:41. The anti-PD-1 antibody and antigen binding fragment thereof may be humanized. A method of treating cancer in a mammalian subject in need thereof is also provided, the method comprising administering to the mammalian subject a therapeutically effective amount of the therapeutic compound. The mammalian subject may be a human. In some embodiments, the cancer is selected from the group consisting of melanoma, non-small cell lung cancer, and renal cell carcinoma.

In accordance with other embodiments, a therapeutic compound of the invention comprises a first protein, the first protein being selected from the group consisting of (i) a CD38 binding peptide consisting of an amino acid sequence that is at least 90% identical to SEQ ID NO:45 and (ii) an anti-CD38 antibody or antigen binding fragment thereof, coupled to an API, the API being an siRNA, the siRNA being a double-stranded RNA molecule including an antisense RNA strand and a sense RNA strand, wherein: (a) the antisense RNA strand is 19-29 nucleotides in length and is complementary to contiguous nucleotides of SEQ ID NO: 2, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In some embodiments, the CD38 binding peptide consists of SEQ ID NO:45. The anti-CD38 antibody and antigen binding fragment thereof may be humanized. A method of treating cancer in a mammalian subject in need thereof is also provided, the method comprising administering to the mammalian subject a therapeutically effective amount of the therapeutic compound. The mammalian subject may be a human. In some embodiments, the cancer is selected from the group consisting of lymphoma, multiple myeloma, and acute myeloid leukemia.

In accordance with some embodiments, a therapeutic compound of the invention comprises a first protein, the first protein being selected from the group consisting of (i) a HER2 binding peptide consisting of an amino acid sequence that is at least 90% identical to a sequence selected from the group consisting of SEQ ID NO: 46-59 and (ii) an anti-HER2 antibody or antigen binding fragment thereof, coupled to an API, the API being an siRNA, the siRNA being a double-stranded RNA molecule including an antisense RNA strand and a sense RNA strand, wherein: (a) the antisense RNA strand is 19-29 nucleotides in length and is complementary to contiguous nucleotides of SEQ ID NO: 3, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In some embodiments, the HER2 binding peptide consists of an amino acid sequence selected from the group consisting of SEQ ID NO: 46-59. The anti-HER2 antibody and antigen binding fragment thereof may be humanized. A method of treating cancer in a mammalian subject in need thereof is also provided, the method comprising administering to the mammalian subject a therapeutically effective amount of the therapeutic compound. The mammalian subject may be a human. In some embodiments, the cancer is selected from the group consisting of breast cancer, ovarian cancer, gastric cancer, and pancreatic cancer.

In accordance with some embodiments, a therapeutic compound of the invention comprises a first protein, the first protein being selected from the group consisting of (i) a PD-L1 binding peptide consisting of an amino acid sequence that is at least 90% identical to a sequence selected from the group consisting of SEQ ID NO: 60-65, (ii) an anti-PD-L1 isoform A antibody or antigen binding fragment thereof, and (iii) an anti-PD-L1 isoform C antibody or antigen binding fragment thereof, coupled to an API, the API being an siRNA, the siRNA being a double-stranded RNA molecule including an antisense RNA strand and a sense RNA strand, wherein: (a) the antisense RNA strand is 19-29 nucleotides in length and is complementary to contiguous nucleotides of SEQ ID NO: 4, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In some embodiments, the PD-L1 binding peptide consists of an amino acid sequence selected from the group consisting of SEQ ID NO: 60-65. The anti-PD-L1 isoform A antibody and antigen binding fragment thereof may be humanized. The anti-PD-L1 isoform C antibody and antigen binding fragment thereof may be humanized. A method of treating cancer in a mammalian subject in need thereof is also provided, the method comprising administering to the mammalian subject a therapeutically effective amount of the therapeutic compound. The mammalian subject may be a human. In some embodiments, the cancer is selected from the group consisting of lung cancer, bladder cancer, and kidney cancer.

In accordance with other embodiments, a therapeutic compound of the invention comprises a first protein, the first protein being selected from the group consisting of (i) an IL4R binding peptide consisting of an amino acid sequence that is at least 90% identical to SEQ ID NO: 66, (ii) an anti-IL4R isoform A antibody or antigen binding fragment thereof, and (iii) an anti-IL4R isoform C antibody or antigen binding fragment thereof, coupled to an API, the API being an siRNA, the siRNA being a double-stranded RNA molecule including an antisense RNA strand and a sense RNA strand, wherein: (a) the antisense RNA strand is 19-29 nucleotides in length and is complementary to contiguous nucleotides of SEQ ID NO: 7, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In some embodiments, the IL4R binding peptide consists of SEQ ID NO: 66. The anti-IL4R isoform A antibody and antigen binding fragment thereof may be humanized. The anti-IL4R isoform C antibody and antigen binding fragment thereof may be humanized. A method of reducing inflammation in a mammalian subject in need thereof is also provided, the method comprising administering to the mammalian subject a therapeutically effective amount of the therapeutic compound. The mammalian subject may be a human.

In accordance with some embodiments, a therapeutic compound of the invention comprises a first protein, the first protein being selected from the group consisting of (i) an IL6R binding peptide consisting of an amino acid sequence that is at least 90% identical to SEQ ID NO: 67, (ii) an anti-IL6R isoform 1 antibody or antigen binding fragment thereof, (iii) an anti-IL6R isoform 2 antibody or antigen binding fragment thereof, (iv) an anti-IL6R isoform 3 antibody or antigen binding fragment thereof, (v) an anti-IL6R isoform 4 antibody or antigen binding fragment thereof, and (vi) an anti-IL6R isoform 5 antibody or antigen binding fragment thereof, coupled to an API, the API being an siRNA, the siRNA being a double-stranded RNA molecule including an antisense RNA strand and a sense RNA strand, wherein: (a) the antisense RNA strand is 19-29 nucleotides in length and is complementary to contiguous nucleotides of SEQ ID NO: 8, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In some embodiments, the IL6R binding peptide consists of SEQ ID NO: 67. The anti-IL6R isoform 1 antibody and antigen binding fragment thereof, the anti-IL6R isoform 2 antibody and antigen binding fragment thereof, the anti-IL6R isoform 3 antibody and antigen binding fragment thereof, the anti-IL6R isoform 4 antibody and antigen binding fragment thereof, and the anti-IL6R isoform 5 antibody and antigen binding fragment thereof, may be humanized. A method of reducing inflammation in a mammalian subject in need thereof is also provided, the method comprising administering to the mammalian subject a therapeutically effective amount of the therapeutic compound. The mammalian subject may be a human.

In accordance with some embodiments, a therapeutic compound of the invention comprises a first protein, the first protein being selected from the group consisting of (i) a TNFR1 binding peptide consisting of an amino acid sequence that is at least 90% identical to SEQ ID NO: 68 and (ii) an anti-TNFR1 antibody or antigen binding fragment thereof, coupled to an API, the API being an siRNA, the siRNA being a double-stranded RNA molecule including an antisense RNA strand and a sense RNA strand, wherein: (a) the antisense RNA strand is 19-29 nucleotides in length and is complementary to contiguous nucleotides of SEQ ID NO: 11, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In some embodiments, the TNFR1 binding peptide consists of SEQ ID NO: 68. The anti-TNFR1 antibody and antigen binding fragment thereof may be humanized. A method of reducing inflammation in a mammalian subject in need thereof is also provided, the method comprising administering to the mammalian subject a therapeutically effective amount of the therapeutic compound. The mammalian subject may be a human.

In accordance with other embodiments, a therapeutic compound of the invention comprises a first protein, the first protein being selected from the group consisting of (i) a TNFR2 binding peptide consisting of an amino acid sequence that is at least 90% identical to a sequence selected from the group consisting of SEQ ID NO: 69-72 and (ii) an anti-TNFR2 antibody or antigen binding fragment thereof, coupled to an API, the API being an siRNA, the siRNA being a double-stranded RNA molecule including an antisense RNA strand and a sense RNA strand, wherein: (a) the antisense RNA strand is 19-29 nucleotides in length and is complementary to contiguous nucleotides of SEQ ID NO: 14, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. In some embodiments, the TNFR2 binding peptide consists of an amino acid sequence selected from the group consisting of SEQ ID NO: 69-72. The anti-TNFR2 antibody and antigen binding fragment thereof may be humanized. A method of reducing inflammation in a mammalian subject in need thereof is also provided, the method comprising administering to the mammalian subject a therapeutically effective amount of the therapeutic compound. The mammalian subject may be a human.

In accordance with some embodiments, a therapeutic compound of the invention comprises glucose coupled to an API, the API being an siRNA, the siRNA being a double-stranded RNA molecule including an antisense RNA strand and a sense RNA strand, wherein: (a) the antisense RNA strand is 19-29 nucleotides in length and is complementary to contiguous nucleotides of a nucleic acid sequence selected from the group consisting of SEQ ID NO: 15, SEQ ID NO: 16, and SEQ ID NO:17, (b) the sense RNA strand is 19-29 nucleotides in length and is complementary to 14-29 nucleotides from the antisense RNA strand, and (c) the double stranded RNA molecule has a double stranded region of 14-29 nucleotides in length and a 3′ overhang region of 0-5 nucleotides in length. A method of treating diabetes in a mammalian subject in need thereof is also provided, the method comprising administering to the mammalian subject a therapeutically effective amount of the therapeutic compound. The mammalian subject may be a human.

### Antisense Oligonucleotides

In accordance with some embodiments, an API may be an antisense oligonucleotide (“ASO”). An antisense oligonucleotide is a single stranded nucleic acid that is complementary to a protein coding messenger mRNA with which it hybridizes, thereby blocking its translation into protein.

In accordance with some embodiments, a therapeutic compound of the invention comprises a first protein, the first protein being selected from the group consisting of (i) a PD-1 binding peptide consisting of an amino acid sequence that is at least 90% identical to a sequence selected from the group consisting of SEQ ID NO:40 and SEQ ID NO:41 and (ii) an anti-PD-1 antibody or antigen binding fragment thereof, coupled to an API, the API being an antisense oligonucleotide of 15-25 nucleotides in length and complementary to at least 15 contiguous nucleotides of SEQ ID NO:1. In some embodiments, the PD-1 binding peptide consists of an amino acid sequence selected from the group consisting of SEQ ID NO:40 and SEQ ID NO:41. The anti-PD-1 antibody and antigen binding fragment thereof may be humanized. A method of treating cancer in a mammalian subject in need thereof is also provided, the method comprising administering to the mammalian subject a therapeutically effective amount of the therapeutic compound. In some embodiments, the cancer is selected from the group consisting of melanoma, non-small cell lung cancer, and renal cell carcinoma.

In accordance with other embodiments, a therapeutic compound of the invention comprises a first protein, the first protein being selected from the group consisting of (i) a CD38 binding peptide consisting of an amino acid sequence that is at least 90% identical to SEQ ID NO:45 and (ii) an anti-CD38 antibody or antigen binding fragment thereof, coupled to an API, the API being an antisense oligonucleotide of 15-25 nucleotides in length and complementary to at least 15 contiguous nucleotides of SEQ ID NO:2. In some embodiments, the CD38 binding peptide consists of SEQ ID NO:45. The anti-CD38 antibody and antigen binding fragment thereof may be humanized. A method of treating cancer in a mammalian subject in need thereof is also provided, the method comprising administering to the subject a therapeutically effective amount of the therapeutic compound. In some embodiments, the cancer is selected from the group consisting of lymphoma, multiple myeloma, and acute myeloid leukemia.

In accordance with some embodiments, a therapeutic compound of the invention comprises a first protein, the first protein being selected from the group consisting of (i) a HER2 binding peptide consisting of an amino acid sequence that is at least 90% identical to a sequence selected from the group consisting of SEQ ID NO: 46-59 and (ii) an anti-HER2 antibody or antigen binding fragment thereof, coupled to an API, the API being an antisense oligonucleotide of 15-25 nucleotides in length and complementary to at least 15 contiguous nucleotides of SEQ ID NO:3. In some embodiments, the HER2 binding peptide consists of an amino acid sequence selected from the group consisting of SEQ ID NO: 46-59. The anti-HER2 antibody and antigen binding fragment thereof may be humanized. A method of treating cancer in a mammalian subject in need thereof is also provided, the method comprising administering to the subject a therapeutically effective amount of the therapeutic compound. In some embodiments, the cancer is selected from the group consisting of breast cancer, ovarian cancer, gastric cancer, and pancreatic cancer.

In accordance with other embodiments, a therapeutic compound of the invention comprises a first protein, the first protein being selected from the group consisting of (i) a PD-L1 binding peptide consisting of an amino acid sequence that is at least 90% identical to a sequence selected from the group consisting of SEQ ID NO: 60-65, (ii) an anti-PD-L1 isoform A antibody or antigen binding fragment thereof, and (iii) an anti-PD-L1 isoform C antibody or antigen binding fragment thereof, coupled to an API, the API being an antisense oligonucleotide of 15-25 nucleotides in length and complementary to at least 15 contiguous nucleotides of SEQ ID NO:4. In some embodiments, the PD-L1 binding peptide consists of an amino acid sequence selected from the group consisting of SEQ ID NO: 60-65. The anti-PD-L1 isoform A antibody and antigen binding fragment thereof may be humanized. The anti-PD-L1 isoform C antibody and antigen binding fragment thereof may be humanized. A method of treating cancer in a mammalian subject in need thereof is also provided, the method comprising administering to the subject a therapeutically effective amount of the therapeutic compound. In some embodiments, the cancer is selected from the group consisting of lung cancer, bladder cancer, and kidney cancer.

In accordance with some embodiments, a therapeutic compound of the invention comprises a first protein, the first protein being selected from the group consisting of (i) an IL4R binding peptide consisting of an amino acid sequence that is at least 90% identical to SEQ ID NO: 66, (ii) an anti-IL4R isoform A antibody or antigen binding fragment thereof, and (iii) an anti-IL4R isoform C antibody or antigen binding fragment thereof, coupled to an API, the API being an antisense oligonucleotide of 15-25 nucleotides in length and complementary to at least 15 contiguous nucleotides of SEQ ID NO:7. In some embodiments, the IL4R binding peptide consists of SEQ ID NO: 66. The anti-IL4R isoform A antibody and antigen binding fragment thereof may be humanized. The anti-IL4R isoform C antibody and antigen binding fragment thereof may be humanized. A method of reducing inflammation in a mammalian subject in need thereof is also provided, the method comprising administering to the subject a therapeutically effective amount of the therapeutic compound.

In accordance with other embodiments, a therapeutic compound of the invention comprises a first protein, the first protein being selected from the group consisting of (i) an IL6R binding peptide consisting of an amino acid sequence that is at least 90% identical to SEQ ID NO: 67, (ii) an anti-IL6R isoform 1 antibody or antigen binding fragment thereof, (iii) an anti-IL6R isoform 2 antibody or antigen binding fragment thereof, (iv) an anti-IL6R isoform 3 antibody or antigen binding fragment thereof, (v) an anti-IL6R isoform 4 antibody or antigen binding fragment thereof, and (vi) an anti-IL6R isoform 5 antibody or antigen binding fragment thereof, coupled to an API, the API being an antisense oligonucleotide of 15-25 nucleotides in length and complementary to at least 15 contiguous nucleotides of SEQ ID NO:8. In some embodiments, the IL6R binding peptide consists of SEQ ID NO: 67. The anti-IL6R isoform 1 antibody and antigen binding fragment thereof, the anti-IL6R isoform 2 antibody and antigen binding fragment thereof, the anti-IL6R isoform 3 antibody and antigen binding fragment thereof, the anti-IL6R isoform 4 antibody and antigen binding fragment thereof, and the anti-IL6R isoform 5 antibody and antigen binding fragment thereof, may be humanized. A method of reducing inflammation in a mammalian subject in need thereof is also provided, the method comprising administering to the subject a therapeutically effective amount of the therapeutic compound.

In accordance with some embodiments, a therapeutic compound of the invention comprises a first protein, the first protein being selected from the group consisting of (i) a TNFR1 binding peptide consisting of an amino acid sequence that is at least 90% identical to SEQ ID NO: 68 and (ii) an anti-TNFR1 antibody or antigen binding fragment thereof, coupled to an API, the API being an antisense oligonucleotide of 15-25 nucleotides in length and complementary to at least 15 contiguous nucleotides of SEQ ID NO:11. In some embodiments, the TNFR1 binding peptide consists of SEQ ID NO: 68. The anti-TNFR1 antibody and antigen binding fragment thereof may be humanized. A method of reducing inflammation in a mammalian subject in need thereof is also provided, the method comprising administering to the subject a therapeutically effective amount of the therapeutic compound.

In accordance with other embodiments, a therapeutic compound of the invention comprises a first protein, the first protein being selected from the group consisting of (i) a TNFR2 binding peptide consisting of an amino acid sequence that is at least 90% identical to a sequence selected from the group consisting of SEQ ID NO: 69-72 and (ii) an anti-TNFR2 antibody or antigen binding fragment thereof, coupled to an API, the API being an antisense oligonucleotide of 15-25 nucleotides in length and complementary to at least 15 contiguous nucleotides of SEQ ID NO:14. In some embodiments, the TNFR2 binding peptide consists of an amino acid sequence selected from the group consisting of SEQ ID NO: 69-72. The anti-TNFR2 antibody and antigen binding fragment thereof may be humanized. A method of reducing inflammation in a mammalian subject in need thereof is also provided, the method comprising administering to the subject a therapeutically effective amount of the therapeutic compound.

In accordance with some embodiments, a therapeutic compound of the invention comprises glucose coupled to an API, the API being an antisense oligonucleotide of 15-25 nucleotides in length and complementary to at least 15 contiguous nucleotides of a nucleic acid sequence selected from the group consisting of SEQ ID NO: 15, SEQ ID NO: 16, and SEQ ID NO:17. A method of treating diabetes in a mammalian subject in need thereof is also provided, the method comprising administering to the subject a therapeutically effective amount of the therapeutic compound.

MicroRNA (“miRNA”)

In accordance with some embodiments, an API may be miRNA. miRNAs are transcribed as single stranded RNA precursors having a stem-loop structure and are subsequently processed as pre-miRNA in the cytosol by the Dicer enzyme, producing one or more mature miRNAs. Typically, two mature miRNA products are produced from an miRNA molecule: a 5p RNA molecule (so named because it is processed from the 5′ arm of the duplex formed as the stem of an miRNA), and a 3p RNA molecule (so named because it is processed from the 3′ arm of the duplex formed as the stem of an miRNA). The 5p and 3p molecules may base pair with each other to form a duplex, and each molecule may be functional—and indeed, may serve a separate function—within a cell through their complementarity to mRNA. Mature miRNA is thought to have regulatory roles, including RNA silencing and post-transcriptional regulation of gene expression.

Table 3, below, provides microRNAs that reduce the expression of the indicated cell surface protein.

TABLE 3
microRNAs for Reducing Expression of Cell Surface Proteins

| | microRNA | Cell Surface Protein |
|---|---|---|
| microRNA | SEQ ID NO | Expression Reduced |
| microRNA 33a | 39 | PD-1, PD-L1 |
| microRNA 708 | 42 | CD38 |
| microRNA 26a-1 | 43 | CD38 |
| microRNA 26a-2 | 44 | CD38 |
| microRNA 552 | 73 | HER2 |
| microRNA 541 | 74 | HER2 |
| microRNA 193a-5p | 75 | HER2 |
| microRNA 134 | 76 | HER2 |
| microRNA 498 | 77 | HER2 |
| microRNA 331-3p | 78 | HER2 |
| microRNA 513 | 79 | PD-L1 |
| microRNA 338-5p | 80 | PD-L1 |
| microRNA 324-5p | 81 | PD-L1 |
| microRNA 152 | 82 | PD-L1 |
| microRNA 200b | 83 | PD-L1 |
| microRNA 138-5p | 84 | PD-L1 |
| microRNA 195 | 85 | PD-L1 |
| microRNA 16 | 86 | PD-L1 |
| microRNA 15a | 87 | PD-L1 |
| microRNA 193a-3p | 88 | PD-L1 |
| microRNA 34a | 89 | PD-L1 |
| microRNA 142 | 90 | PD-L1 |
| microRNA 424 | 91 | PD-L1 |
| microRNA 497-5p | 92 | PD-L1 |
| microRNA 140 | 93 | PD-L1 |
| microRNA 340 | 94 | PD-L1, IL-4R |
| microRNA 383 | 95 | PD-L1 |
| microRNA 17-5p | 96 | PD-L1 |
| microRNA 155 | 97 | PD-L1 |
| microRNA 429 | 98 | IL-4R |
| microRNA 511 | 99 | TNFR 1 |
| microRNA 29a-3p | 100 | TNFR1 |
| microRNA 181c | 101 | TNFR1 |
| microRNA 187 | 102 | TNFR1 |

In accordance with some embodiments, a therapeutic compound of the invention comprises a first protein, the first protein being selected from the group consisting of (i) a PD-1 binding peptide consisting of an amino acid sequence that is at least 90% identical to a sequence selected from the group consisting of SEQ ID NO:40 and SEQ ID NO:41 and (ii) an anti-PD-1 antibody or antigen binding fragment thereof, coupled to an API, the API being a microRNA consisting of a nucleic acid sequence at least 95% identical to SEQ ID NO: 39. In some embodiments, the PD-1 binding peptide consists of an amino acid sequence selected from the group consisting of SEQ ID NO:40 and SEQ ID NO:41. The anti-PD-1 antibody and antigen binding fragment thereof may be humanized. In some embodiments, the microRNA consists of SEQ ID NO: 39. A method of treating cancer in a mammalian subject in need thereof is also provided, the method comprising administering to the subject a therapeutically effective amount of the therapeutic compound. In some embodiments, the cancer is selected from the group consisting of melanoma, non-small cell lung cancer, and renal cell carcinoma.

In accordance with other embodiments, a therapeutic compound of the invention comprises a first protein, the first protein being selected from the group consisting of (i) a CD38 binding peptide consisting of an amino acid sequence that is at least 90% identical to SEQ ID NO:45 and (ii) an anti-CD38 antibody or antigen binding fragment thereof, coupled to an API, the API being a microRNA consisting of a nucleic acid sequence at least 95% identical to a microRNA sequence selected from the group consisting of SEQ ID NO: 42-44. In some embodiments, the CD38 binding peptide consists of SEQ ID NO:45. The anti-CD38 antibody and antigen binding fragment thereof may be humanized. In some embodiments, the microRNA consists of a nucleic acid sequence selected from the group consisting of SEQ ID NO: 42-44. A method of treating cancer in a mammalian subject in need thereof is also provided, the method comprising administering to the subject a therapeutically effective amount of the therapeutic compound. In some embodiments, the cancer is selected from the group consisting of lymphoma, multiple myeloma, and acute myeloid leukemia.

In accordance with other embodiments, a therapeutic compound of the invention comprises a first protein, the first protein being selected from the group consisting of (i) a HER2 binding peptide consisting of an amino acid sequence that is at least 90% identical to a sequence selected from the group consisting of SEQ ID NO:46-59 and (ii) an anti-HER2 antibody or antigen binding fragment thereof, coupled to an API, the API being a microRNA consisting of a nucleic acid sequence at least 95% identical to a microRNA sequence selected from the group consisting of SEQ ID NO: 73-78. In some embodiments, the HER2 binding peptide consists an amino acid sequence selected from the group consisting of SEQ ID NO:46-59. The anti-HER2 antibody and antigen binding fragment thereof may be humanized. In some embodiments, the microRNA consists of a nucleic acid sequence selected from the group consisting of SEQ ID NO: 73-78. A method of treating cancer in a mammalian subject in need thereof is also provided, the method comprising administering to the subject a therapeutically effective amount of the therapeutic compound. In some embodiments, the cancer is selected from the group consisting of breast cancer, ovarian cancer, gastric cancer, and pancreatic cancer.

In accordance with other embodiments, a therapeutic compound of the invention comprises a first protein, the first protein being selected from the group consisting of (i) a PD-L1 binding peptide consisting of an amino acid sequence that is at least 90% identical to a sequence selected from the group consisting of SEQ ID NO:60-65, (ii) an anti-PD-L1 isoform A antibody or antigen binding fragment thereof, and (iii) an anti-PD-L1 isoform C antibody or antigen binding fragment thereof, coupled to an API, the API being a microRNA consisting of a nucleic acid sequence at least 95% identical to a microRNA sequence selected from the group consisting of SEQ ID NO: 39, 79-96, and 97. In some embodiments, the PD-L1 binding peptide consists an amino acid sequence selected from the group consisting of SEQ ID NO:60-65. The anti-PD-L1 isoform A antibody and antigen binding fragment thereof may be humanized. The anti-PD-L1 isoform C antibody and antigen binding fragment thereof may be humanized. In some embodiments, the microRNA consists of a nucleic acid sequence selected from the group consisting of SEQ ID NO: 39, 79-96, and 97. A method of treating cancer in a mammalian subject in need thereof is also provided, the method comprising administering to the subject a therapeutically effective amount of the therapeutic compound. In some embodiments, the cancer is selected from the group consisting of lung cancer, bladder cancer, and kidney cancer.

In accordance with other embodiments, a therapeutic compound of the invention comprises a first protein, the first protein being selected from the group consisting of (i) an IL4R binding peptide consisting of an amino acid sequence that is at least 90% identical to SEQ ID NO:66, (ii) an anti-IL4R isoform A antibody or antigen binding fragment thereof, and (iii) an anti-IL4R isoform A antibody or antigen binding fragment thereof, coupled to an API, the API being a microRNA consisting of a nucleic acid sequence at least 95% identical to a microRNA sequence selected from the group consisting of SEQ ID NO: 94 and SEQ ID NO: 98. In some embodiments, the IL4R binding peptide consists of SEQ ID NO:66. The anti-IL4R isoform A antibody and antigen binding fragment thereof may be humanized. The anti-IL4R isoform C antibody and antigen binding fragment thereof may be humanized. In some embodiments, the microRNA consists of a nucleic acid sequence selected from the group consisting of SEQ ID NO: 94 and SEQ ID NO: 98. A method of reducing inflammation in a mammalian subject in need thereof is also provided, the method comprising administering to the subject a therapeutically effective amount of the therapeutic compound.

In accordance with other embodiments, a therapeutic compound of the invention comprises a first protein, the first protein being selected from the group consisting of (i) a TNFR1 binding peptide consisting of an amino acid sequence that is at least 90% identical SEQ ID NO:68 and (ii) an anti-TNFR1 antibody or antigen binding fragment thereof, coupled to an API, the API being a microRNA consisting of a nucleic acid sequence at least 95% identical to a microRNA sequence selected from the group consisting of SEQ ID NO: 99-102. In some embodiments, the TNFR1 binding peptide consists of SEQ ID NO:68. The anti-TNFR1 antibody and antigen binding fragment thereof may be humanized. In some embodiments, the microRNA consists of a nucleic acid sequence selected from the group consisting of SEQ ID NO: 99-102. A method of reducing inflammation in a mammalian subject in need thereof is also provided, the method comprising administering to the subject a therapeutically effective amount of the therapeutic compound.

### Pharmaceutical Compositions and Methods of Administration

Therapeutic compounds as described herein can be formulated into pharmaceutical compositions using methods available in the art and those disclosed herein. Any of the therapeutic compounds disclosed herein can be provided in the appropriate pharmaceutical composition and be administered by a suitable route of administration.

The methods provided herein encompass administering pharmaceutical compositions containing at least one therapeutic compound as described herein, either used alone or in the form of a combination with one or more compatible and pharmaceutically acceptable carriers, such as diluents or adjuvants.

In clinical practice, therapeutic compounds provided herein may be administered by any conventional route, in particular orally, parenterally, rectally, or by inhalation (e.g. in the form of aerosols).

Use may be made, as liquid compositions for oral administration, of solutions which are pharmaceutically acceptable, suspensions, emulsions, syrups and elixirs containing inert diluents, such as water or liquid paraffin. These compositions can also comprise substances other than diluents, for example wetting, sweetening or flavoring products.

The compositions for parenteral administration can be emulsions or sterile solutions. Use may be made, as solvent or vehicle, of propylene glycol, a polyethylene glycol, vegetable oils, in particular olive oil, or injectable organic esters, for example ethyl oleate. These compositions can also contain adjuvants, in particular wetting, isotonizing, emulsifying, dispersing, and stabilizing agents. Sterilization can be carried out in several ways, for example using a bacteriological filter or by radiation.

In certain embodiments, a composition provided herein is a pharmaceutical composition or a single unit dosage form. Pharmaceutical compositions and single unit dosage forms provided herein comprise a prophylactically or therapeutically effective amount of a therapeutic compound provided herein and, typically, one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific active ingredients in the dosage form. The composition or single unit dosage form, if desired, can also contain minor amounts of wetting or emulsifying agents, or pH buffering agents.

Further provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, and the like. The formulation should suit the mode of administration. In a certain embodiment, the pharmaceutical compositions or single unit dosage forms are sterile and in suitable form for administration to a mammalian subject.

A pharmaceutical composition is formulated to be compatible with its intended route of administration. Examples of routes of administration include, but are not limited to, parenteral, e.g., intravenous, intradermal, subcutaneous, intramuscular, subcutaneous, oral, buccal, sublingual, inhalation, intranasal, transdermal, topical, transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to human beings. In an embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol.

Compounds that increase the solubility of one or more of the active ingredients disclosed herein can also be incorporated into the parenteral dosage forms.

### Example 1: Construction of PD-L1 Binding Peptide-siRNA Conjugate

A PD-L1 binding peptide-siRNA conjugate was prepared using azide and DBCO click chemistry. An azide functionalized PD-L1 binding peptide (SEQ ID NO:60) ((Azidoacetic acid)-NYSKPTDRQYHF) and an anti-PD-L1 siRNA (having a dibenzocyclooctyne (DBCO) functionalized sense strand (SEQ ID NO:12) (GACUCAAGAUGGAACCUGAdTdT-[DBCO]) and a Cy5.5-labeled antisense strand (SEQ ID NO:13) (dTdTCUGAGUUCUACCUUGGACU-Cy5.5)) were mixed in a 2:1 molar ratio, respectively. 10 μl of Cy5.5-labeled siRNA at a concentration of 100 of μM was mixed with 2 nmol of the azide functionalized PD-L1 binding peptide in a total volume of 10.33 μ1 RNAse-free water and incubated at 37° C. in a thermomixer for 2 hours at 1100 rpm.

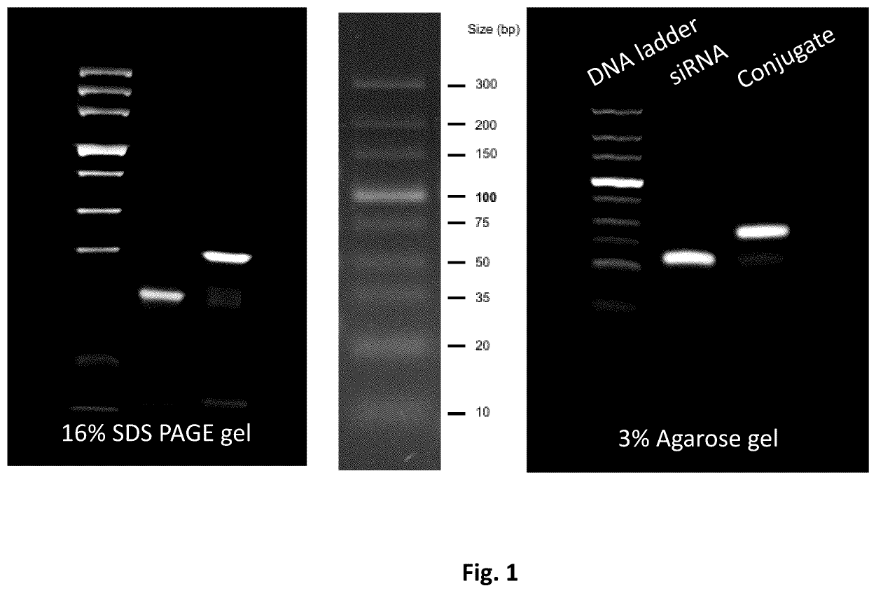

After incubation, the conjugation was verified via a 3% TBE agarose gel (visualized using SYBR-safe) and a 16% SDS PAGE gel (stained with ethidium bromide). As shown in FIG. 1, the reaction successfully produced the desired PD-L1 binding peptide-siRNA conjugate.

### Example 2: PD-L1 Binding Peptide-siRNA Conjugate Binds Specifically to PD-L1

We tested whether the PD-L1 binding peptide-siRNA conjugate of Example 1 was able to specifically bind PD-L1 expressed by CT26.CL25 cells by incubating the PD-L1 binding peptide-siRNA conjugate of Example 1 with CT26.CL25 cells that either had, or had not, been pre-treated with an anti-PD-L1 antibody. As a control, CT26.CL25 cells were incubated with the unconjugated anti-PD-L1 siRNA of Example 1.

Here, 2 ml of CT26.CL25 cells (at 5×10⁶ cells/ml) were seeded into each of four dishes. 24 hours after seeding, and one hour before treatment with the conjugate of Example 1, one dish was pre-treated with 1 ml of anti-PD-L1 antibody (InVivoMAb anti-mouse PD-L1, BioXCell Cat. #BE0101) at 1 mg/ml. After incubation with the antibody, the pre-treated cells were incubated with 1 ml (200 nM) of the conjugate of Example 1 for 30 minutes at 4° C. At the same time, a dish that had not been pre-treated was similarly incubated with the conjugate of Example 1, and another dish that had not been pre-treated was similarly incubated with the unconjugated anti-PD-L1 siRNA of Example 1.

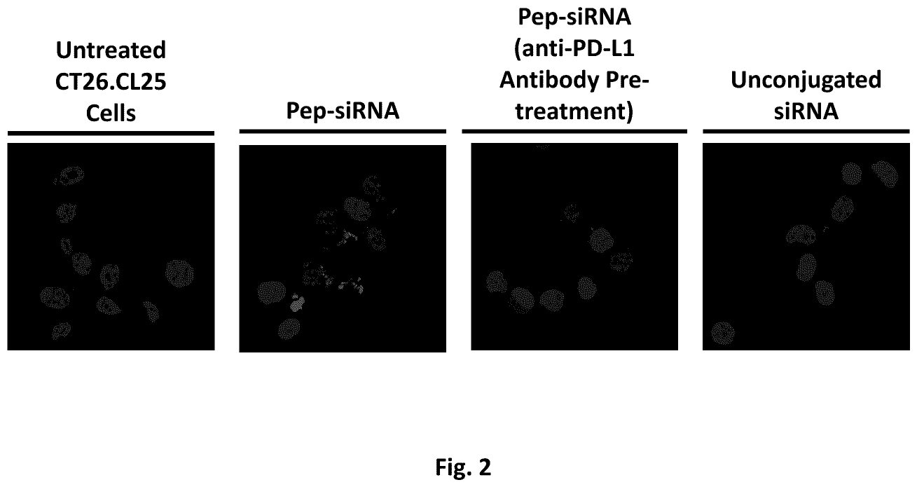

Cells were then fixated with 4% paraformaldehyde (PFA) for 10 min, then washed twice with DPBS. Nuclei were then stained using Hoechst 33342 (blue of FIG. 2) and the cells were examined for Cy5.5 fluorescence (red of FIG. 2).

As shown in FIG. 2, untreated cells demonstrate no Cy5.5 fluorescence, whereas cells treated only with the conjugate of Example 1 (“Pep-siRNA”) show significant Cy5.5 fluorescence. However, cells that were pre-treated with the anti-PD-L1 antibody followed by treatment with the conjugate of Example 1, and cells that were treated only with the unconjugated Cy5.5-labeled siRNA of Example 1, show very low levels of Cy5.5 fluorescence.

These results demonstrate that the conjugate of Example 1 specifically binds to PD-L1 on CT26.CL25 cells and that this binding can be blocked by an anti-PD-L1 antibody. Furthermore, these results suggest that binding is due to the PD-L1 binding peptide of the conjugate and not the siRNA.

### Example 3: PD-L1 Binding Peptide-siRNA Conjugate is Internalized after Binding to PD-L1

CT26.CL25 cells were incubated, as described in Example 2, with either the PD-L1 binding peptide-siRNA conjugate of Example 1 or the unconjugated Cy5.5-labeled siRNA of Example 1. After 9 hours, 18 hours, and 24 hours of incubation, cells were then fixated with 4% paraformaldehyde (PFA) for 10 min and washed twice with DPBS.

B16F10 cells were also incubated with either the PD-L1 binding peptide-siRNA conjugate of Example 1 or the unconjugated Cy5.5-labeled siRNA of Example 1 and similarly fixated after 18 hours and 24 hours of incubation.

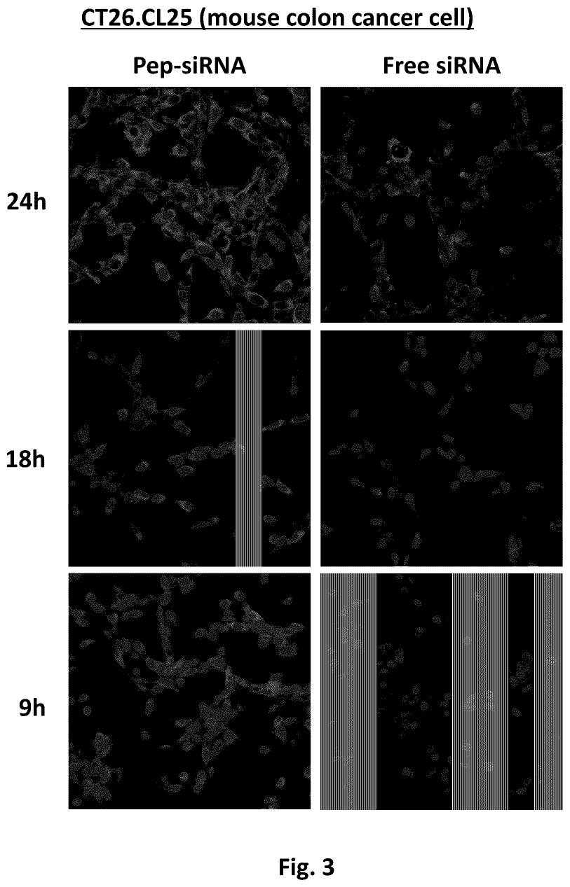

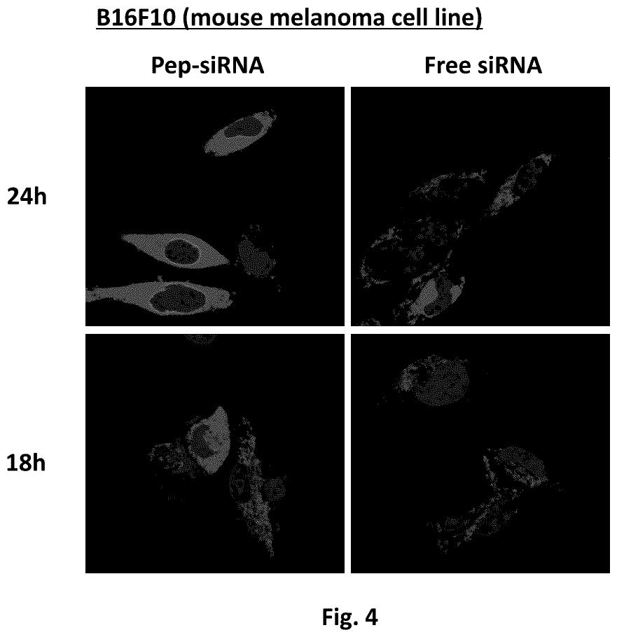

Nuclei were stained using Hoechst 33342 (blue of FIGS. 3 and 4) and the cells were examined for Cy5.5 fluorescence (red of FIGS. 3 and 4).

As shown in FIGS. 3 and 4, CT26.CL25 and B16F10 cells treated with the conjugate of Example 1 internalized the conjugate, the amount of internalization increasing with increased incubation time. In contrast, CT26.CL25 and B16F10 cells treated with unconjugated Cy5.5-labeled siRNA show significantly less Cy5.5 fluorescence, indicating a much lower amount of siRNA internalization.

### Example 4: PD-L1 Binding Peptide-siRNA Conjugate Silences PD-L1 mRNA and PD-L1 Protein Expression

CT26.CL25 cells were incubated, as described in Example 2, with the PD-L1 binding peptide-siRNA conjugate of Example 1. After 24 hours, RNA was extracted from the cells using a Qiagen RNeasy Plus kit according to the manufacturer's protocol. RNA from untreated CT26.CL25 cells was also extracted.

After the RNA extraction, cDNA was made using oligodT(20mer) (SEQ ID NO: 127) and Bioneer RT-PCR kit.

qRT-PCR was also performed on the extracted RNA using SYBR-green from Enzynomics. Six qRT-PCR reactions were run on each sample.

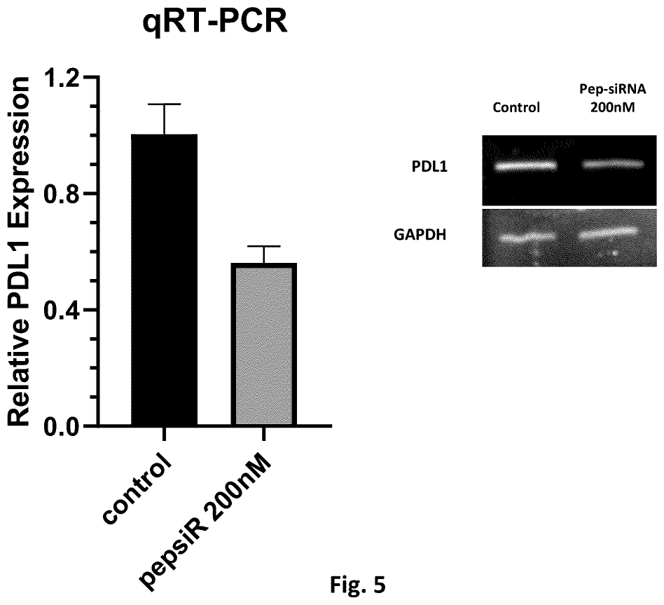

GAPDH expression was used as a control. The following primers were used for both qRT-PCR and RT-PCR (shown in the gel of FIG. 5) of GAPDH and PD-L1:

a. GAPDH:
(SEQ ID NO: 33)
CCACCCAGAAGACTGTGGAT
and
(SEQ ID NO: 34)
CACATTGGGGGTAGGAACAC.
b. PD-L1:
(SEQ ID NO: 103)
GCTCCAAAGGACTTGTACGTG
and
(SEQ ID NO: 104)
TGATCTGAAGGGCAGCATTTC.

For the gel analyzing the RT-PCR products, 1% TBE gel was used to visualize the RT-PCR bands. The RT-PCR was as follows: initial denaturation 95° C. 5 min, denaturation 95° C. 10 sec, annealing 60° C. 15 sec, elongation 72° C. 15 sec, final elongation 72° C. 30 sec (20 cycles for GAPDH, 30 cycles for PD-L1).

As can be seen in the gel of FIG. 5, cells treated with the conjugate of Example 1 expressed less PD-L1 mRNA, as evidenced by the fainter RT-PCR cDNA band compared to the untreated control cells. No difference in band intensity is observed for GAPDH RT-PCR products.

Moreover, the qRT-PCR results shown in FIG. 5 demonstrate a significant reduction of PD-L1 expression in conjugate-treated cells versus untreated cells, indicating that the conjugate of Example 1 is capable of silencing PD-L1 RNA expression in CT26.CL25 cells after being internalized.

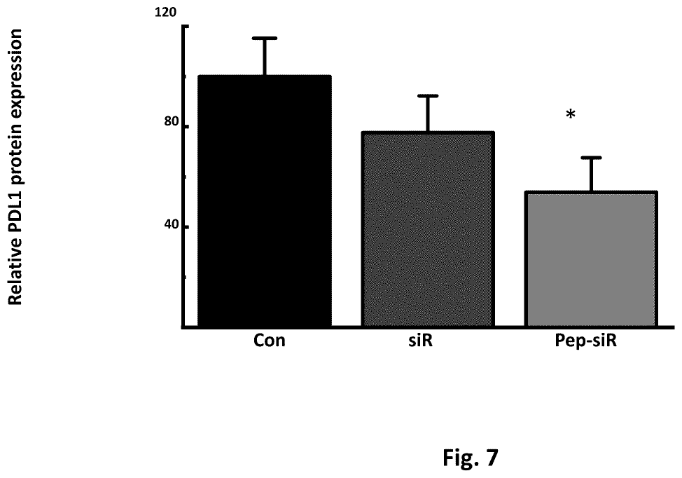

Finally, as shown in FIG. 7, as determined via Western blot, CT6.CL25 cells treated with the conjugate of Example 1 (Pep-siR) express less PD-L1 protein than untreated CT6.CL25 cells and CT6.CL25 cells treated with only the siRNA (siR) of the conjugate of Example 1.

### Example 5: Treatment with PD-L1 Binding Peptide-siRNA Conjugate Reduces Anti-PD-L1 Antibody Binding of 4T1 Cells More than Treatment with PD-L1 Binding Peptide Alone

2 ml of 4T1 cells (a breast cancer cell line derived from the mammary gland tissue of a mouse BALB/c strain) were seeded into each well of a 6-well plate at a concentration of 2.5×10⁶ cells/ml. After 24 hours, the cells were treated with either 1 ml (500 pmol/ml) of the PD-L1 binding peptide-siRNA conjugate of Example 1, or with 1 ml (500 pmol/ml) of unconjugated PD-L1 binding protein (SEQ ID NO:60) ((Azidoacetic acid)-NYSKPTDRQYHF), and incubated at 37° C. for 48 hours. An untreated group of cells was used as a control.

After the incubation, cells were detached from the plate using TE. The cells were then washed and resuspended at a concentration of 10⁵ cells/ml in DPBS for each treatment group and the control.

Each group of cells was then treated with an allophycocyanin (APC)-labeled anti-PD-L1 mouse monoclonal antibody (BioLegend Cat. #124312). For each treatment group, 10 μ1 of a 1:500 dilution of the APC-labeled anti-PD-L1 antibody was added to 100 μ1 of the resuspended cells and the cells were incubated in 110 μ1 for 30 minutes at 4° C.

After incubation, the cells were centrifuged and resuspended in [what media] twice in order to wash off any unbound antibody.

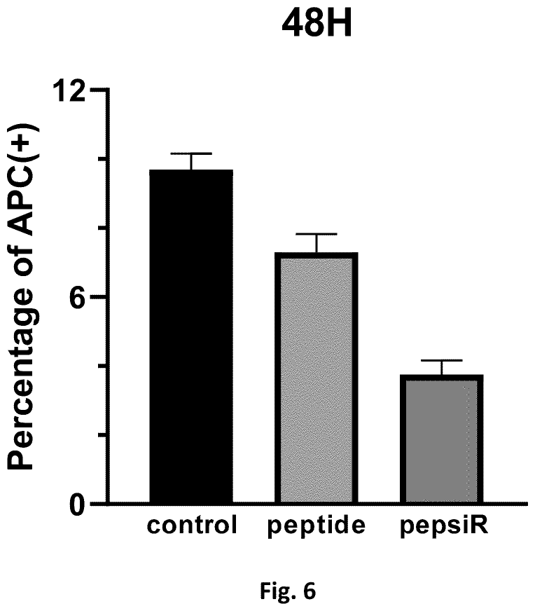

Each group of cells, as well as the control, was then analyzed using fluorescence-activated cell sorting (638 nm laser, channel of 660/10 nm), the results of which are shown in FIG. 6. As can be seen, treatment of 4T1 cells with PD-L1 binding peptide reduces subsequent binding of the APC-labeled antibody compared to the untreated control, indicating that treatment with PD-L1 binding protein blocks PD-L1 on the cell surface and/or causes a reduction of the amount of PD-L1 on the cell surface through receptor-mediated endocytosis.

Meanwhile, the treatment with the PD-L1 binding peptide-siRNA conjugate reduces the subsequent binding of the APC-labeled antibody to a greater degree compared to both the untreated control and the PD-L1 binding protein treatment group. The difference in percentage of APC-labeled antibody binding strongly suggests that, apart from blocking PD-L1 on the cell surface and/or causing a reduction of the amount of PD-L1 on the cell surface through receptor-mediated endocytosis, the conjugate also silences expression PD-L1 expression through RNA interference.

### Example 6: Construction of EGFR Binding Peptide-siRNA Conjugate

An EGFR binding peptide-siRNA conjugate was prepared in a method similar to the conjugate described in Example 1. An azide functionalized EGFR binding peptide ((Azidoacetic acid)-YHWYGYTPQNVI (SEQ ID NO: 119)) and an anti-EGFR siRNA (having a dibenzocyclooctyne (DBCO) functionalized sense strand (SEQ ID NO:120) (AUAGGCAUUGGUGAAUUUAAAGAdCdA-[DBCO]) and an antisense strand (SEQ ID NO:121) (UGUCUUUAAAUUCACCAAUGCCUAUGC)) were mixed in a 2:1 molar ratio, respectively. 10 μl of siRNA at a concentration of 100 μM was mixed with 2 nmol of the azide functionalized EGFR binding peptide in a total volume of 10.33 μ1 RNAse-free water and incubated at 37° C. in a thermomixer for 2 hours at 1100 rpm.

After incubation, the conjugation was verified via a 3% TBE agarose gel (visualized using SYBR-safe) and a 16% SDS PAGE gel (stained with ethidium bromide).

### Example 7: EGFR Binding Peptide-siRNA Conjugate Binds Specifically to EGFR

We tested whether the EGFR binding peptide-siRNA conjugate of Example 6 was able to specifically bind EGFR expressed by A549 cells by incubating the EGFR binding peptide-siRNA conjugate of Example 6 with A549 cells. As a control, A549 cells were incubated with the unconjugated anti-EGFR siRNA of Example 6.

A549 cells were treated with 200 nM of either the EGFR-binding peptide-conjugated siRNA of Example 6 or the unconjugated EGFR siRNA of Example 6, each stained with YOYO-1, a nucleic acid staining dye.

After 30 minutes, cells were then fixated with 4% paraformaldehyde (PFA) for 10 minutes, then washed twice with DPBS. Nuclei were then stained using Hoechst 33342 and the cells were examined for YOYO-1 staining.

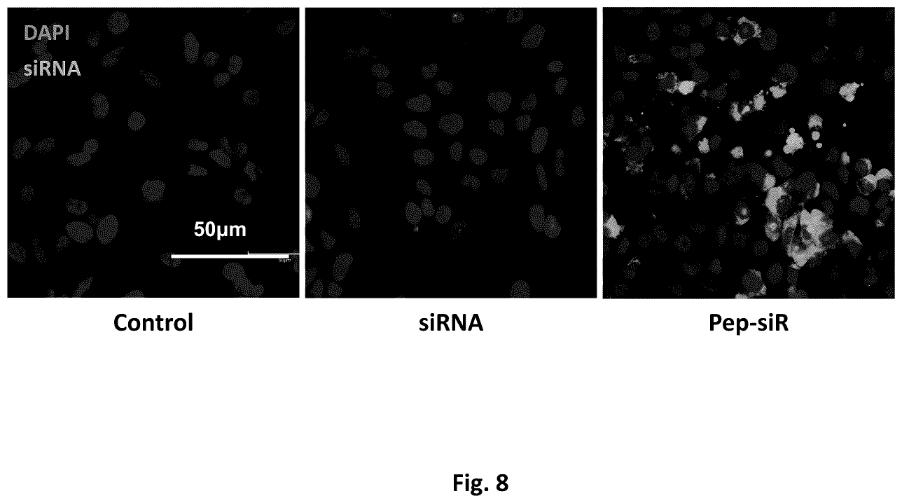

As shown in FIG. 8, untreated cells (control), and cells treated with siRNA alone (siRNA), demonstrate no YOYO-1 staining, whereas cells treated with the conjugate of Example 6 (Pep-siR) show significant YOYO-1 staining.

These results demonstrate that the conjugate of Example 6 specifically binds to EGFR of A549 cells.

### Example 8: EGFR Binding Peptide-siRNA Conjugate Silences EGFR mRNA and EGFR Protein Expression

A549 cells were incubated, similar to that described in Example 2 for the PD-L1 binding peptide-siRNA conjugate, with the EGFR binding peptide-siRNA conjugate of Example 6. After 24 hours, RNA was extracted from the cells using a Qiagen RNeasy Plus kit according to the manufacturer's protocol. RNA from untreated A549 cells was also extracted.

After the RNA extraction, cDNA was made using oligodT(20mer) (SEQ ID NO: 127) and Bioneer RT-PCR kit.

qRT-PCR was also performed on the extracted RNA using SYBR-green from Enzynomics. Six qRT-PCR reactions were run on each sample.

GAPDH expression was used as a control. The following primers were used for both qRT-PCR and RT-PCR of GAPDH and EGFR:

a. GAPDH:
(SEQ ID NO: 33)
CCACCCAGAAGACTGTGGAT
and
(SEQ ID NO: 34)
CACATTGGGGGTAGGAACAC.
b. EGFR:
(SEQ ID NO: 122)
TCCCTCAGCCACCCATATGTAC
and
(SEQ ID NO: 123)
GTCTCGGGCCATTTTGGAGAATCC.

Relative expression was determined by gel electrophoresis. For the gel analyzing the RT-PCR products, 1% TBE gel was used to visualize the RT-PCR bands. The RT-PCR was as follows: initial denaturation 95° C. 5 min, denaturation 95° C. 10 sec, annealing 60° C. 15 sec, elongation 72° C. 15 sec, final elongation 72° C. 30 sec (20 cycles for GAPDH, 30 cycles for PD-L1).

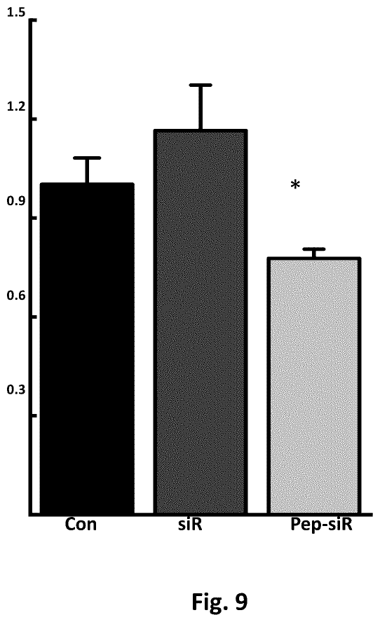

As shown in the qRT-PCR bar chart results of FIG. 9, cells treated with the conjugate of Example 6 expressed less EGFR mRNA than untreated cells and cells treated with unconjugated EGFR siRNA, indicating that the conjugate of Example 6 silences EGFR RNA expression in A549 cells.

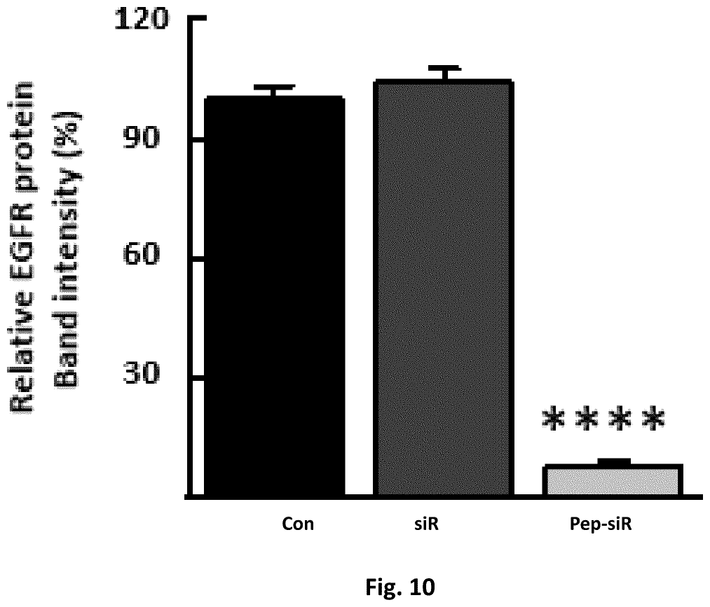

Moreover, as shown in FIG. 10, as determined via Western blot, A549 cells treated with the conjugate of Example 6 (Pep-siR) express less EGFR protein than untreated A549 cells and A549 cells treated with only the siRNA (siR) of the conjugate of Example 6.

Various embodiments of the present invention may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

Without limitation, potential subject matter that may be claimed (prefaced with the letter “P” so as to avoid confusion with the actual claims presented below) includes:
P1. A therapeutic compound comprising:
   a conjugate configured to bind to a second protein expressed on a surface of a target cell, the conjugate comprising a first protein coupled to an API,
   wherein:
the conjugate is configured to bind to the second protein expressed on the surface of the target cell in a manner so as to inhibit an activity of the second protein,
the conjugate is further configured to be internalized by the target cell upon binding to the second protein expressed on the surface of the target cell,
the API is configured to be released from the conjugate after the conjugate has been internalized by the target cell, and
the API is further configured to reduce the expression of the second protein expressed on the surface of the target cell after the API has been released from the conjugate, thereby further inhibiting the activity of the second protein,
so that the conjugate and the API synergistically inhibit the activity of the second protein.
P2. The therapeutic compound of potential claim P1, wherein the API is coupled to the first protein by a linker.
P3. The therapeutic compound of potential claim P2, wherein the linker is a cleavable linker.
P4. The therapeutic compound according to any one of the preceding potential claims, wherein the second protein consists of a sequence selected from the group consisting of SEQ ID NOs: 18, 19, 20, 21, 22, 23, 24, 25, 26, 27, 29, 31, 32, 35, 36, 37, and 38.
P5. The therapeutic compound according to any one of potential claims P1-P3, wherein the second protein consists of a second protein amino acid sequence at least 90% identical to a sequence selected from the group consisting of SEQ ID NOs: 18, 19, 20, 21, 22, 23, 24, 25, 26, 27, 29, 31, 32, 35, 36, 37, and 38.
P6. The therapeutic compound according to any one of the preceding potential claims, wherein the API is selected from the group consisting of a siRNA, an antisense oligonucleotide, and a microRNA.
P7. The therapeutic compound according to any one of potential claims P1-P3, wherein the second protein is PD-1 (SEQ ID NO:18) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 1,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P8. The therapeutic compound according to any one of potential claims P1-P3, wherein the second protein consists of a second protein amino acid sequence at least 90% identical to PD-1 (SEQ ID NO:18) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 1,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P9. The therapeutic compound according to any one of potential claims P1-3, wherein the second protein is PD-1 (SEQ ID NO:18) and wherein the API is a microRNA, the microRNA consisting of a nucleic acid sequence, the nucleic acid sequence being at least 95% identical to SEQ ID NO: 39.
P10. The therapeutic compound according to any one of potential claims P1-3, wherein the second protein is PD-1 (SEQ ID NO:18) and wherein the API is a microRNA, the microRNA consisting of SEQ ID NO: 39.
P11. The therapeutic compound according to any one of potential claims P1-3, wherein the second protein consists of a second protein amino acid sequence at least 90% identical to PD-1 (SEQ ID NO:18) and wherein the API is a microRNA, the microRNA consisting of a nucleic acid sequence, the nucleic acid sequence being at least 95% identical to SEQ ID NO: 39.
P12. The therapeutic compound according to any one of potential claims P1-3, wherein the second protein consists of a second protein amino acid sequence at least 90% identical to PD-1 (SEQ ID NO:18) and wherein the API is a microRNA, the microRNA consisting of SEQ ID NO: 39.
P13. The therapeutic compound according to any one of potential claims P1-3, wherein the second protein is PD-1 (SEQ ID NO:18) and wherein the API is an antisense oligonucleotide, the antisense oligonucleotide being 15-25 nucleotides in length and complementary to at least 15 contiguous nucleotides of SEQ ID NO:1.
P14. The therapeutic compound according to any one of potential claims P1-3 and P7-13, wherein the first protein is selected from the group consisting of an anti-PD-1 antibody and an antigen-binding fragment thereof.
P15. The therapeutic compound according to any one of potential claims P1-3 and P7-13, wherein the first protein is a PD-1 binding peptide consisting of an amino acid sequence, the amino acid sequence being at least 90% identical to a peptide sequence selected from the group consisting of SEQ ID NO:40 and SEQ ID NO:41.
P16. The therapeutic compound according to any one of potential claims P1-3 and P7-13, wherein the first protein is a PD-1 binding peptide consisting of an amino acid sequence selected from the group consisting of SEQ ID NO:40 and SEQ ID NO:41.
P17. The therapeutic compound according to any one of potential claims P1-3, wherein the second protein is CD38 (SEQ ID NO:19) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 2,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P18. The therapeutic compound according to any one of potential claims P1-3, wherein the second protein consists of a second protein amino acid sequence at least 90% identical to CD38 (SEQ ID NO:19) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 2,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P19. The therapeutic compound according to any one of potential claims P1-3, wherein the second protein is CD38 (SEQ ID NO:19) and wherein the API is a microRNA, the microRNA consisting of a nucleic acid sequence, the nucleic acid sequence being at least 95% identical to a microRNA sequence selected from the group consisting of SEQ ID NO:42-44.
P20. The therapeutic compound according to any one of potential claims P1-3, wherein the second protein consists of a second protein amino acid sequence at least 90% identical to CD38 (SEQ ID NO:19) and wherein the API is a microRNA, the microRNA consisting of a nucleic acid sequence, the nucleic acid sequence being at least 95% identical to a microRNA sequence selected from the group consisting of SEQ ID NO:42-44.
P21. The therapeutic compound according to any one of potential claims P1-3, wherein the second protein is CD38 (SEQ ID NO:19) and wherein the API is a microRNA consisting of a nucleic acid sequence selected from the group consisting of SEQ ID NO:42-44.
P22. The therapeutic compound according to any one of potential claims P1-3, wherein the second protein consists of a second protein amino acid sequence at least 90% identical to CD38 (SEQ ID NO:19) and wherein the API is a microRNA consisting of a nucleic acid sequence selected from the group consisting of SEQ ID NO:42-44.
P23. The therapeutic compound according to any one of potential claims P1-3, wherein the second protein is CD38 (SEQ ID NO:19) and wherein the API is an antisense oligonucleotide, the antisense oligonucleotide being 15-25 nucleotides in length and complementary to at least 15 contiguous nucleotides of SEQ ID NO:2.
P24. The therapeutic compound according to any one of potential claims P1-3, wherein the second protein consists of a second protein amino acid sequence at least 90% identical to CD38 (SEQ ID NO:19) and wherein the API is an antisense oligonucleotide, the antisense oligonucleotide being 15-25 nucleotides in length and complementary to at least 15 contiguous nucleotides of SEQ ID NO:2.
P25. The therapeutic compound according to any one of potential claims P1-3 and P17-24, wherein the first protein is selected from the group consisting of an anti-CD38 antibody and an antigen-binding fragment thereof.
P26. The therapeutic compound according to any one of potential claims P1-3 and P17-24, wherein the first protein is a CD38 binding peptide consisting of an amino acid sequence, the amino acid sequence being at least 90% identical to SEQ ID NO:45.
P27. The therapeutic compound according to any one of potential claims P1-3 and P17-24, wherein the first protein is a CD38 binding peptide consisting of SEQ ID NO:45.
P28. The therapeutic compound according to any one of potential claims P1-3, wherein the second protein is HER2 (SEQ ID NO:20) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 3,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P29. The therapeutic compound according to any one of potential claims P1-3, wherein the second protein consists of a second protein amino acid sequence at least 90% identical to HER2 (SEQ ID NO:20) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 3,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P30. The therapeutic compound according to any one of potential claims P1-3, wherein the second protein is HER2 (SEQ ID NO:20) and wherein the API is a microRNA, the microRNA consisting of a nucleic acid sequence, the nucleic acid sequence being at least 95% identical to a microRNA sequence selected from the group consisting of SEQ ID NO:73-78.
P31. The therapeutic compound according to any one of potential claims P1-3, wherein the second protein consists of a second protein amino acid sequence at least 90% identical to HER2 (SEQ ID NO:20) and wherein the API is a microRNA, the microRNA consisting of a nucleic acid sequence, the nucleic acid sequence being at least 95% identical to a microRNA sequence selected from the group consisting of SEQ ID NO:73-78.
P32. The therapeutic compound according to any one of potential claims P1-3, wherein the second protein is HER2 (SEQ ID NO:20) and wherein the API is a microRNA consisting of a nucleic acid sequence selected from the group consisting of SEQ ID NO:73-78.
P33. The therapeutic compound according to any one of potential claims P1-3, wherein the second protein consists of a second protein amino acid sequence at least 90% identical to HER2 (SEQ ID NO:20) and wherein the API is a microRNA consisting of a nucleic acid sequence selected from the group consisting of SEQ ID NO:73-78.
P34. The therapeutic compound according to any one of potential claims P1-3, wherein the second protein is HER2 (SEQ ID NO:20) and wherein the API is an antisense oligonucleotide, the antisense oligonucleotide being 15-25 nucleotides in length and complementary to at least 15 contiguous nucleotides of SEQ ID NO:3.
P35. The therapeutic compound according to any one of potential claims P1-3, wherein the second protein consists of a second protein amino acid sequence at least 90% identical to HER2 (SEQ ID NO:20) and wherein the API is an antisense oligonucleotide, the antisense oligonucleotide being 15-25 nucleotides in length and complementary to at least 15 contiguous nucleotides of SEQ ID NO:3.
P36. The therapeutic compound according to any one of potential claims P1-3 and P28-35, wherein the first protein is selected from the group consisting of an anti-HER2 antibody and an antigen-binding fragment thereof.
P37. The therapeutic compound according to any one of potential claims P1-3 and P28-35, wherein the first protein is a HER2 binding peptide consisting of an amino acid sequence, the amino acid sequence being at least 90% identical to a peptide sequence selected from the group consisting of SEQ ID NO:46-59.
P38. The therapeutic compound according to any one of potential claims P1-3 and P28-35, wherein the first protein is a HER2 binding peptide consisting of an amino acid sequence selected from the group consisting of SEQ ID NO:46-59.
P39. The therapeutic compound according to any one of potential claims P1-3, wherein the second protein is PD-L1 isoform A (SEQ ID NO:21) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 4,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P40. The therapeutic compound according to any one of potential claims P1-3, wherein the second protein consists of a second protein amino acid sequence at least 90% identical to PD-L1 isoform A (SEQ ID NO:21) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 4,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P41. The therapeutic compound according to any one of potential claims P1-3, wherein the second protein is PD-L1 isoform C (SEQ ID NO:22) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 5,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P42. The therapeutic compound according to any one of potential claims P1-3, wherein the second protein consists of a second protein amino acid sequence at least 90% identical to PD-L1 isoform C (SEQ ID NO:22) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 5,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P43. The therapeutic compound according to any one of potential claims P1-3, wherein the second protein is PD-L1 isoform A (SEQ ID NO:21) and wherein the API is a microRNA, the microRNA consisting of a nucleic acid sequence, the nucleic acid sequence being at least 95% identical to a microRNA sequence selected from the group consisting of SEQ ID NO:39, 79-96, and 97.
P44. The therapeutic compound according to any one of potential claims P1-3, wherein the second protein consists of a second protein amino acid sequence at least 90% identical to PD-L1 isoform A (SEQ ID NO:21) and wherein the API is a microRNA, the microRNA consisting of a nucleic acid sequence, the nucleic acid sequence being at least 95% identical to a microRNA sequence selected from the group consisting of SEQ ID NO:39, 79-96, and 97.
P45. The therapeutic compound according to any one of potential claims P1-3, wherein the second protein is PD-L1 isoform A (SEQ ID NO:21) and wherein the API is a microRNA consisting of a nucleic acid sequence selected from the group consisting of SEQ ID NO: 39, 79-96, and 97.
P46. The therapeutic compound according to any one of potential claims P1-3, wherein the second protein consists of a second protein amino acid sequence at least 90% identical to PD-L1 isoform A (SEQ ID NO:21) and wherein the API is a microRNA consisting of a nucleic acid sequence selected from the group consisting of SEQ ID NO: 39, 79-96, and 97
P47. The therapeutic compound according to any one of potential claims P1-3, wherein the second protein is PD-L1 isoform C (SEQ ID NO:22) and wherein the API is a microRNA, the microRNA consisting of a nucleic acid sequence, the nucleic acid sequence being at least 95% identical to a microRNA sequence selected from the group consisting of SEQ ID NO: 39, 79-96, and 97.
P48. The therapeutic compound according to any one of potential claims P1-3, wherein the second protein consists of a second protein amino acid sequence at least 90% identical to PD-L1 isoform C (SEQ ID NO:22) and wherein the API is a microRNA, the microRNA consisting of a nucleic acid sequence, the nucleic acid sequence being at least 95% identical to a microRNA sequence selected from the group consisting of SEQ ID NO: 39, 79-96, and 97.
P49. The therapeutic compound according to any one of potential claims P1-3, wherein the second protein is PD-L1 isoform C (SEQ ID NO:22) and wherein the API is a microRNA consisting of a nucleic acid sequence selected from the group consisting of SEQ ID NO: 39, 79-96, and 97.
P50. The therapeutic compound according to any one of potential claims P1-3, wherein the second protein consists of a second protein amino acid sequence at least 90% identical to PD-L1 isoform C (SEQ ID NO:22) and wherein the API is a microRNA consisting of a nucleic acid sequence selected from the group consisting of SEQ ID NO: 39, 79-96, and 97.
P51. The therapeutic compound according to any one of potential claims P1-3, wherein the second protein is PD-L1 isoform A (SEQ ID NO:21) and wherein the API is an antisense oligonucleotide, the antisense oligonucleotide being 15-25 nucleotides in length and complementary to at least 15 contiguous nucleotides of SEQ ID NO:4.
P52. The therapeutic compound according to any one of potential claims P1-3, wherein the second protein consists of a second protein amino acid sequence at least 90% identical to PD-L1 isoform A (SEQ ID NO:21) and wherein the API is an antisense oligonucleotide, the antisense oligonucleotide being 15-25 nucleotides in length and complementary to at least 15 contiguous nucleotides of SEQ ID NO:4.
P53. The therapeutic compound according to any one of potential claims P1-3, wherein the second protein is PD-L1 isoform C (SEQ ID NO:22) and wherein the API is an antisense oligonucleotide, the antisense oligonucleotide being 15-25 nucleotides in length and complementary to at least 15 contiguous nucleotides of SEQ ID NO:5.
P54. The therapeutic compound according to any one of potential claims P1-3, wherein the second protein consists of a second protein amino acid sequence at least 90% identical to PD-L1 isoform C (SEQ ID NO:22) and wherein the API is an antisense oligonucleotide, the antisense oligonucleotide being 15-25 nucleotides in length and complementary to at least 15 contiguous nucleotides of SEQ ID NO:5.
P55. The therapeutic compound according to any one of potential claims P1-3, P39, P40,
P43, P44, P45, P46, P51, and P52, wherein the first protein is selected from the group consisting of an anti-PD-L1 isoform A antibody and an antigen-binding fragment thereof.
P56. The therapeutic compound according to any one of potential claims P1-3, P41, P42,
P47, P48, P49, P50, P53, and P54, wherein the first protein is selected from the group consisting of an anti-PD-L1 isoform C antibody and an antigen-binding fragment thereof.
P57. The therapeutic compound according to any one of potential claims P1-3 and P39-53, wherein the first protein is a PD-L1 binding peptide consisting of an amino acid sequence, the amino acid sequence being at least 90% identical to a peptide sequence selected from the group consisting of SEQ ID NO:60-65.
P58. The therapeutic compound according to any one of potential claims P1-3 and P39-53, wherein the first protein is a PD-L1 binding peptide consisting of an amino acid sequence selected from the group consisting of SEQ ID NO:60-65.
P59. The therapeutic compound according to any one of potential claims P1-3, wherein the second protein is IL4R isoform A (SEQ ID NO:23) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 6,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P60. The therapeutic compound according to any one of potential claims P1-3, wherein the second protein consists of a second protein amino acid sequence at least 90% identical to IL4R isoform A (SEQ ID NO:23) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 6,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P61. The therapeutic compound according to any one of potential claims P1-3, wherein the second protein is IL4R isoform C (SEQ ID NO:24) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 7,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P62. The therapeutic compound according to any one of potential claims P1-3, wherein the second protein consists of a second protein amino acid sequence at least 90% identical to IL4R isoform C (SEQ ID NO:24) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 7,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P63. The therapeutic compound according to any one of potential claims P1-3, wherein the second protein is IL4R isoform A (SEQ ID NO:23) and wherein the API is a microRNA, the microRNA consisting of a nucleic acid sequence, the nucleic acid sequence being at least 95% identical to a microRNA sequence selected from the group consisting of SEQ ID NO:94 and 98.
P64. The therapeutic compound according to any one of potential claims P1-3, wherein the second protein consists of a second protein amino acid sequence at least 90% identical to IL4R isoform A (SEQ ID NO:23) and wherein the API is a microRNA, the microRNA consisting of a nucleic acid sequence, the nucleic acid sequence being at least 95% identical to a microRNA sequence selected from the group consisting of SEQ ID NO:94 and 98.
P65. The therapeutic compound according to any one of potential claims P1-3, wherein the second protein is IL4R isoform A (SEQ ID NO:23) and wherein the API is a microRNA consisting of a nucleic acid sequence selected from the group consisting of SEQ ID NO: 94 and 98.
P66. The therapeutic compound according to any one of potential claims P1-3, wherein the second protein consists of a second protein amino acid sequence at least 90% identical to IL4R isoform A (SEQ ID NO:23) and wherein the API is a microRNA consisting of a nucleic acid sequence selected from the group consisting of SEQ ID NO: 94 and 98.
P67. The therapeutic compound according to any one of potential claims P1-3, wherein the second protein is IL4R isoform C (SEQ ID NO:24) and wherein the API is a microRNA, the microRNA consisting of a nucleic acid sequence, the nucleic acid sequence being at least 95% identical to a microRNA sequence selected from the group consisting of SEQ ID NO: 94 and 98.
P68. The therapeutic compound according to any one of potential claims P1-3, wherein the second protein consists of a second protein amino acid sequence at least 90% identical to IL4R isoform C (SEQ ID NO:24) and wherein the API is a microRNA, the microRNA consisting of a nucleic acid sequence, the nucleic acid sequence being at least 95% identical to a microRNA sequence selected from the group consisting of SEQ ID NO: 94 and 98.
P69. The therapeutic compound according to any one of potential claims P1-3, wherein the second protein is IL4R isoform C (SEQ ID NO:24) and wherein the API is a microRNA consisting of a nucleic acid sequence selected from the group consisting of SEQ ID NO: 94 and 98.
P70. The therapeutic compound according to any one of potential claims P1-3, wherein the second protein consists of a second protein amino acid sequence at least 90% identical to IL4R isoform C (SEQ ID NO:24) and wherein the API is a microRNA consisting of a nucleic acid sequence selected from the group consisting of SEQ ID NO: 94 and 98.
P71. The therapeutic compound according to any one of potential claims P1-3, wherein the second protein is IL4R isoform A (SEQ ID NO:23) and wherein the API is an antisense oligonucleotide, the antisense oligonucleotide being 15-25 nucleotides in length and complementary to at least 15 contiguous nucleotides of SEQ ID NO:6.
P72. The therapeutic compound according to any one of potential claims P1-3, wherein the second protein consists of a second protein amino acid sequence at least 90% identical to IL4R isoform A (SEQ ID NO:23) and wherein the API is an antisense oligonucleotide, the antisense oligonucleotide being 15-25 nucleotides in length and complementary to at least 15 contiguous nucleotides of SEQ ID NO:6.
P73. The therapeutic compound according to any one of potential claims P1-3, wherein the second protein is IL4R isoform C (SEQ ID NO:24) and wherein the API is an antisense oligonucleotide, the antisense oligonucleotide being 15-25 nucleotides in length and complementary to at least 15 contiguous nucleotides of SEQ ID NO:7.
P74. The therapeutic compound according to any one of potential claims P1-3, wherein the second protein consists of a second protein amino acid sequence at least 90% identical to IL4R isoform C (SEQ ID NO:24) and wherein the API is an antisense oligonucleotide, the antisense oligonucleotide being 15-25 nucleotides in length and complementary to at least 15 contiguous nucleotides of SEQ ID NO:7.
P75. The therapeutic compound according to any one of potential claims P1-3, P59, P60, P63, P64, P65, P66, P71, and P72, wherein the first protein is selected from the group consisting of an anti-IL4R isoform A antibody and an antigen-binding fragment thereof.
P76. The therapeutic compound according to any one of potential claims P1-3, P61, P62, P67, P68, P69, P70, P73, and P74, wherein the first protein is selected from the group consisting of an anti-IL4R isoform C antibody and an antigen-binding fragment thereof.
P77. The therapeutic compound according to any one of potential claims P1-3 and P59-74, wherein the first protein is a IL4R binding peptide consisting of an amino acid sequence at least 90% identical to SEQ ID NO:66.
P78. The therapeutic compound according to any one of potential claims P1-3 and P59-74, wherein the first protein is a IL4R binding peptide consisting of SEQ ID NO:66.
P79. The therapeutic compound according to any one of potential claims P1-3, wherein the second protein is IL6R isoform 1 (SEQ ID NO:25) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 8,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P80. The therapeutic compound according to any one of potential claims P1-3, wherein the second protein consists of a second protein amino acid sequence at least 90% identical to IL6R isoform 1 (SEQ ID NO:25) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 8,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P81. The therapeutic compound according to any one of potential claims P1-3, wherein the second protein is IL6R isoform 2 (SEQ ID NO:26) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 9,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P82. The therapeutic compound according to any one of potential claims P1-3, wherein the second protein consists of a second protein amino acid sequence at least 90% identical to IL6R isoform 2 (SEQ ID NO:26) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 9,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P83. The therapeutic compound according to any one of potential claims P1-3, wherein the second protein is IL6R isoform 3 (SEQ ID NO:27) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 10,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P84. The therapeutic compound according to any one of potential claims P1-3, wherein the second protein consists of a second protein amino acid sequence at least 90% identical to IL6R isoform 3 (SEQ ID NO:27) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 10,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P85. The therapeutic compound according to any one of potential claims P1-3, wherein the second protein is IL6R isoform 4 (SEQ ID NO:29) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 28,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P86. The therapeutic compound according to any one of potential claims P1-3, wherein the second protein consists of a second protein amino acid sequence at least 90% identical to IL6R isoform 4 (SEQ ID NO:29) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 28,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P87. The therapeutic compound according to any one of potential claims P1-3, wherein the second protein is IL6R isoform 5 (SEQ ID NO:31) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 30,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P88. The therapeutic compound according to any one of potential claims P1-3, wherein the second protein consists of a second protein amino acid sequence at least 90% identical to IL6R isoform 5 (SEQ ID NO:31) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 30,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P89. The therapeutic compound according to any one of potential claims P1-3, wherein the second protein is IL6R isoform 1 (SEQ ID NO:25) and wherein the API is an antisense oligonucleotide, the antisense oligonucleotide being 15-25 nucleotides in length and complementary to at least 15 contiguous nucleotides of SEQ ID NO:8.
P90. The therapeutic compound according to any one of potential claims P1-3, wherein the second protein consists of a second protein amino acid sequence at least 90% identical to IL6R isoform 1 (SEQ ID NO:25) and wherein the API is an antisense oligonucleotide, the antisense oligonucleotide being 15-25 nucleotides in length and complementary to at least 15 contiguous nucleotides of SEQ ID NO:8.
P91. The therapeutic compound according to any one of potential claims P1-3, wherein the second protein is IL6R isoform 2 (SEQ ID NO:26) and wherein the API is an antisense oligonucleotide, the antisense oligonucleotide being 15-25 nucleotides in length and complementary to at least 15 contiguous nucleotides of SEQ ID NO:9.
P92. The therapeutic compound according to any one of potential claims P1-3, wherein the second protein consists of a second protein amino acid sequence at least 90% identical to IL6R isoform 2 (SEQ ID NO:26) and wherein the API is an antisense oligonucleotide, the antisense oligonucleotide being 15-25 nucleotides in length and complementary to at least 15 contiguous nucleotides of SEQ ID NO:9.
P93. The therapeutic compound according to any one of potential claims P1-3, wherein the second protein is IL6R isoform 3 (SEQ ID NO:27) and wherein the API is an antisense oligonucleotide, the antisense oligonucleotide being 15-25 nucleotides in length and complementary to at least 15 contiguous nucleotides of SEQ ID NO:10.
P94. The therapeutic compound according to any one of potential claims P1-3, wherein the second protein consists of a second protein amino acid sequence at least 90% identical to IL6R isoform 3 (SEQ ID NO:27) and wherein the API is an antisense oligonucleotide, the antisense oligonucleotide being 15-25 nucleotides in length and complementary to at least 15 contiguous nucleotides of SEQ ID NO:10.
P95. The therapeutic compound according to any one of potential claims P1-3, wherein the second protein is IL6R isoform 4 (SEQ ID NO:29) and wherein the API is an antisense oligonucleotide, the antisense oligonucleotide being 15-25 nucleotides in length and complementary to at least 15 contiguous nucleotides of SEQ ID NO:28.
P96. The therapeutic compound according to any one of potential claims P1-3, wherein the second protein consists of a second protein amino acid sequence at least 90% identical to IL6R isoform 4 (SEQ ID NO:29) and wherein the API is an antisense oligonucleotide, the antisense oligonucleotide being 15-25 nucleotides in length and complementary to at least 15 contiguous nucleotides of SEQ ID NO:28.
P97. The therapeutic compound according to any one of potential claims P1-3, wherein the second protein is IL6R isoform 5 (SEQ ID NO:31) and wherein the API is an antisense oligonucleotide, the antisense oligonucleotide being 15-25 nucleotides in length and complementary to at least 15 contiguous nucleotides of SEQ ID NO:30.
P98. The therapeutic compound according to any one of potential claims P1-3, wherein the second protein consists of a second protein amino acid sequence at least 90% identical to IL6R isoform 5 (SEQ ID NO:31) and wherein the API is an antisense oligonucleotide, the antisense oligonucleotide being 15-25 nucleotides in length and complementary to at least 15 contiguous nucleotides of SEQ ID NO:30.
P99. The therapeutic compound according to any one of potential claims P1-3, P79-80, and P89-90, wherein the first protein is selected from the group consisting of an anti-IL6R isoform 1 antibody and an antigen-binding fragment thereof.
P100. The therapeutic compound according to any one of potential claims P1-3, P81-82, and P91-92, wherein the first protein is selected from the group consisting of an anti-IL6R isoform 2 antibody and an antigen-binding fragment thereof.
P101. The therapeutic compound according to any one of potential claims P1-3, P83-84, and P93-94, wherein the first protein is selected from the group consisting of an anti-IL6R isoform 3 antibody and an antigen-binding fragment thereof.
P102. The therapeutic compound according to any one of potential claims P1-3, P85-86, and P95-96, wherein the first protein is selected from the group consisting of an anti-IL6R isoform 4 antibody and an antigen-binding fragment thereof.
P103. The therapeutic compound according to any one of potential claims P1-3, P87-88, and P97-98, wherein the first protein is selected from the group consisting of an anti-IL6R isoform 5 antibody and an antigen-binding fragment thereof.
P104. The therapeutic compound according to any one of potential claims P1-3 and P79-98, wherein the first protein is a IL6R binding peptide consisting of an amino acid sequence at least 90% identical to SEQ ID NO:67.
P105. The therapeutic compound according to any one of potential claims P1-3 and P79-98, wherein the first protein is a IL6R binding peptide consisting of SEQ ID NO:67.
P106. The therapeutic compound according to any one of potential claims P1-3, wherein the second protein is TNFR1 (SEQ ID NO:32) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 11,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P107. The therapeutic compound according to any one of potential claims P1-3, wherein the second protein consists of a second protein amino acid sequence at least 90% identical to TNFR1 (SEQ ID NO:32) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 11,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P108. The therapeutic compound according to any one of potential claims P1-3, wherein the second protein is TNFR1 (SEQ ID NO:32) and wherein the API is a microRNA, the microRNA consisting of a nucleic acid sequence, the nucleic acid sequence being at least 95% identical to a microRNA sequence selected from the group consisting of SEQ ID NO:99-102.
P109. The therapeutic compound according to any one of potential claims P1-3, wherein the second protein consists of a second protein amino acid sequence at least 90% identical to TNFR1 (SEQ ID NO:32) and wherein the API is a microRNA, the microRNA consisting of a nucleic acid sequence, the nucleic acid sequence being at least 95% identical to a microRNA sequence selected from the group consisting of SEQ ID NO:99-102.
P110. The therapeutic compound according to any one of potential claims P1-3, wherein the second protein is TNFR1 (SEQ ID NO:32) and wherein the API is a microRNA consisting of a nucleic acid sequence selected from the group consisting of SEQ ID NO:99-102.
P111. The therapeutic compound according to any one of potential claims P1-3, wherein the second protein consists of a second protein amino acid sequence at least 90% identical to TNFR1 (SEQ ID NO:32) and wherein the API is a microRNA consisting of a nucleic acid sequence selected from the group consisting of SEQ ID NO:99-102.
P112. The therapeutic compound according to any one of potential claims P1-3, wherein the second protein is TNFR1 (SEQ ID NO:32) and wherein the API is an antisense oligonucleotide, the antisense oligonucleotide being 15-25 nucleotides in length and complementary to at least 15 contiguous nucleotides of SEQ ID NO:11.
P113. The therapeutic compound according to any one of potential claims P1-3, wherein the second protein consists of a second protein amino acid sequence at least 90% identical to TNFR1 (SEQ ID NO:32) and wherein the API is an antisense oligonucleotide, the antisense oligonucleotide being 15-25 nucleotides in length and complementary to at least 15 contiguous nucleotides of SEQ ID NO:11.
P114. The therapeutic compound according to any one of potential claims P1-3 and P68-71, wherein the first protein is selected from the group consisting of an anti-TNFR1 antibody and an antigen-binding fragment thereof.
P115. The therapeutic compound according to any one of potential claims P1-3 and P106-113, wherein the first protein is a TNFR1 binding peptide consisting of an amino acid sequence, the amino acid sequence being at least 90% identical to SEQ ID NO:68.
P116. The therapeutic compound according to any one of potential claims P1-3 and P106-113, wherein the first protein is a TNFR1 binding peptide consisting of SEQ ID NO:68.
P117. The therapeutic compound according to any one of potential claims P1-3, wherein the second protein is TNFR2 (SEQ ID NO:35) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 14,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P118. The therapeutic compound according to any one of potential claims P1-3, wherein the second protein consists of a second protein amino acid sequence at least 90% identical to TNFR2 (SEQ ID NO:35) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 14,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P119. The therapeutic compound according to any one of potential claims P1-3, wherein the second protein is TNFR2 (SEQ ID NO:35) and wherein the API is an antisense oligonucleotide, the antisense oligonucleotide being 15-25 nucleotides in length and complementary to at least 15 contiguous nucleotides of SEQ ID NO:14.
P120. The therapeutic compound according to any one of potential claims P1-3, wherein the second protein consists of a second protein amino acid sequence at least 90% identical to TNFR2 (SEQ ID NO:35) and wherein the API is an antisense oligonucleotide, the antisense oligonucleotide being 15-25 nucleotides in length and complementary to at least 15 contiguous nucleotides of SEQ ID NO:14.
P121. The therapeutic compound according to any one of potential claims P1-3 and P117-120, wherein the first protein is selected from the group consisting of an anti-TNFR2 antibody and an antigen-binding fragment thereof.
P122. The therapeutic compound according to any one of potential claims P1-3 and P117-120, wherein the first protein is a TNFR2 binding peptide consisting of an amino acid sequence, the amino acid sequence being at least 90% identical to a peptide sequence selected from the group consisting of SEQ ID NO:69-72.
P123. The therapeutic compound according to any one of potential claims P1-3 and P117-120, wherein the first protein is a TNFR2 binding peptide consisting of an amino acid sequence selected from the group consisting of SEQ ID NO:69-72.
P124. A therapeutic compound comprising:
   a conjugate configured to bind to a sodium-dependent glucose cotransporter expressed on a surface of a target cell, the conjugate comprising a glucose coupled to an API,
   wherein:
the conjugate is configured to bind to the sodium-dependent glucose cotransporter so as to be transported across a membrane of the target cell and into the target cell,
the API is configured to be released from the conjugate after the conjugate has been transported across the membrane of the target cell and into the target cell, and
the API is further configured to reduce the expression of the sodium-dependent glucose cotransporter expressed on the surface of the target cell after the API has been released from the conjugate.
P125. The therapeutic compound of potential claim P124, wherein the API is coupled to the glucose by a linker.
P126. The therapeutic compound of potential claim P125, wherein the linker is a cleavable linker.
P127. The therapeutic compound according to any one potential claims P124-126, wherein the API is selected from the group consisting of a siRNA and an antisense oligonucleotide.
P128. The therapeutic compound according to any one of potential claims P124-126, wherein the sodium-dependent glucose cotransporter is SGLT1 isoform 1 (SEQ ID NO:36) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 15,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P129. The therapeutic compound according to any one of potential claims P124-126, wherein the sodium-dependent glucose cotransporter consists of an amino acid sequence at least 90% identical to SGLT1 isoform 1 (SEQ ID NO:36) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 15,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P130. The therapeutic compound according to any one of potential claims P124-126, wherein the sodium-dependent glucose cotransporter is SGLT1 isoform 1 (SEQ ID NO:36) and wherein the API is an antisense oligonucleotide, the antisense oligonucleotide being 15-25 nucleotides in length and complementary to at least 15 contiguous nucleotides of SEQ ID NO:15.
P131. The therapeutic compound according to any one of potential claims P124-126, wherein the sodium-dependent glucose cotransporter consists of an amino acid sequence at least 90% identical to SGLT1 isoform 1 (SEQ ID NO:36) and wherein the API is an antisense oligonucleotide, the antisense oligonucleotide being 15-25 nucleotides in length and complementary to at least 15 contiguous nucleotides of SEQ ID NO:15.
P132. The therapeutic compound according to any one of potential claims P124-126, wherein the sodium-dependent glucose cotransporter is SGLT1 isoform 2 (SEQ ID NO:37) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 16,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P133. The therapeutic compound according to any one of potential claims P124-126, wherein the sodium-dependent glucose cotransporter consists of an amino acid sequence at least 90% identical to SGLT1 isoform 2 (SEQ ID NO:37) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 16,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P134. The therapeutic compound according to any one of potential claims P124-126, wherein the sodium-dependent glucose cotransporter is SGLT1 isoform 2 (SEQ ID NO:37) and wherein the API is an antisense oligonucleotide, the antisense oligonucleotide being 15-25 nucleotides in length and complementary to at least 15 contiguous nucleotides of SEQ ID NO:16.
P135. The therapeutic compound according to any one of potential claims P124-126, wherein the sodium-dependent glucose cotransporter consists of an amino acid sequence at least 90% identical to SGLT1 isoform 2 (SEQ ID NO:37) and wherein the API is an antisense oligonucleotide, the antisense oligonucleotide being 15-25 nucleotides in length and complementary to at least 15 contiguous nucleotides of SEQ ID NO:16.
P136. The therapeutic compound according to any one of potential claims P124-126, wherein the sodium-dependent glucose cotransporter is SGLT2 (SEQ ID NO:38) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 17,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P137. The therapeutic compound according to any one of potential claims P124-126, wherein the sodium-dependent glucose cotransporter consists of an amino acid sequence at least 90% identical to SGLT2 (SEQ ID NO:38) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 17,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.
P138. The therapeutic compound according to any one of potential claims P124-126, wherein the sodium-dependent glucose cotransporter is SGLT2 (SEQ ID NO:38) and wherein the API is an antisense oligonucleotide, the antisense oligonucleotide being 15-25 nucleotides in length and complementary to at least 15 contiguous nucleotides of SEQ ID NO:17.
P139. The therapeutic compound according to any one of potential claims P124-126, wherein the sodium-dependent glucose cotransporter consists of an amino acid sequence at least 90% identical to SGLT2 (SEQ ID NO:38) and wherein the API is an antisense oligonucleotide, the antisense oligonucleotide being 15-25 nucleotides in length and complementary to at least 15 contiguous nucleotides of SEQ ID NO:17.
P140. A method of treating cancer in a mammalian subject in need thereof, the method comprising administering to the mammalian subject a therapeutically effective amount of the therapeutic compound according to any one of potential claims P1-58.
P141. A method of reducing inflammation in a mammalian subject in need thereof, the method comprising administering to the mammalian subject a therapeutically effective amount of the therapeutic compound according to any one of potential claims P59-123.
P142. A method of treating diabetes in a mammalian subject in need thereof, the method comprising administering to the mammalian subject a therapeutically effective amount of the therapeutic compound according to any one of potential claims P124-139.
P143. The method according to any one of potential claims P140-142, wherein the mammalian subject is a human.
P144. A pharmaceutical composition comprising the therapeutic compound according to any one of potential claims P1-139 and a pharmaceutically acceptable carrier.

The publications (including patent publications), web sites, company names, books, manuals, treatise, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A therapeutic compound comprising:
a conjugate configured to bind to a second protein expressed on a surface of a target cell, the conjugate comprising a first protein coupled to an API,
wherein:
the conjugate is configured to bind to the second protein expressed on the surface of the target cell in a manner so as to inhibit an activity of the second protein,
the conjugate is further configured to be internalized by the target cell upon binding to the second protein expressed on the surface of the target cell,
the API is configured to be released from the conjugate after the conjugate has been internalized by the target cell, and
the API is further configured to reduce the expression of the second protein expressed on the surface of the target cell after the API has been released from the conjugate, thereby further inhibiting the activity of the second protein,
so that the conjugate and the API synergistically inhibit the activity of the second protein.

2. The therapeutic compound according to claim 1, wherein the second protein is PD-1 (SEQ ID NO:18) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 1,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.

3. The therapeutic compound according to claim 1, wherein the second protein is PD-1 (SEQ ID NO:18) and wherein the API is a microRNA, the microRNA consisting of a nucleic acid sequence, the nucleic acid sequence being at least 95% identical to SEQ ID NO: 39.

4. The therapeutic compound of claim 1, wherein the second protein is PD-1 (SEQ ID NO:18) and wherein the API is an antisense oligonucleotide, the antisense oligonucleotide being 15-25 nucleotides in length and complementary to at least 15 contiguous nucleotides of SEQ ID NO:1.

5. The therapeutic compound of claim 1, wherein the first protein is a PD-1 binding peptide consisting of an amino acid sequence, the amino acid sequence being at least 90% identical to a peptide sequence selected from the group consisting of SEQ ID NO:40 and SEQ ID NO:41.

6. The therapeutic compound of claim 1, wherein the second protein is HER2 (SEQ ID NO:20) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 3,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.

7. The therapeutic compound of claim 1, wherein the second protein is HER2 (SEQ ID NO:20) and wherein the API is a microRNA, the microRNA consisting of a nucleic acid sequence, the nucleic acid sequence being at least 95% identical to a microRNA sequence selected from the group consisting of SEQ ID NO:73-78.

8. The therapeutic compound of claim 1, wherein the second protein is HER2 (SEQ ID NO:20) and wherein the API is a microRNA consisting of a nucleic acid sequence selected from the group consisting of SEQ ID NO:73-78.

9. The therapeutic compound of claim 1, wherein the second protein is HER2 (SEQ ID NO:20) and wherein the API is an antisense oligonucleotide, the antisense oligonucleotide being 15-25 nucleotides in length and complementary to at least 15 contiguous nucleotides of SEQ ID NO:3.

10. The therapeutic compound of claim 1, wherein the first protein is a HER2 binding peptide consisting of an amino acid sequence, the amino acid sequence being at least 90% identical to a peptide sequence selected from the group consisting of SEQ ID NO:46-59.

11. The therapeutic compound according to claim 1, wherein the second protein is PD-L1 isoform A (SEQ ID NO:21) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 4,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.

12. The therapeutic compound of claim 1, wherein the second protein is PD-L1 isoform C (SEQ ID NO:22) and wherein the API is an siRNA, the siRNA being a double-stranded RNA molecule including an antisense RNA strand and a sense RNA strand,
wherein:
(a) the antisense RNA strand is 19-29 nucleotides in length and is complementary to contiguous nucleotides of SEQ ID NO: 5,
(b) the sense RNA strand is 19-29 nucleotides in length and is complementary to 14-29 nucleotides from the antisense RNA strand, and
(c) the double stranded RNA molecule has a double stranded region of 14-29 nucleotides in length and a 3′ overhang region of 0-5 nucleotides in length.

13. The therapeutic compound of claim 1, wherein the second protein is PD-L1 isoform A (SEQ ID NO:21) and wherein the API is a microRNA, the microRNA consisting of a nucleic acid sequence, the nucleic acid sequence being at least 95% identical to a microRNA sequence selected from the group consisting of SEQ ID NO:39, 79-96, and 97.

14. The therapeutic compound of claim 1, wherein the second protein is PD-L1 isoform A (SEQ ID NO:21) and wherein the API is a microRNA consisting of a nucleic acid sequence selected from the group consisting of SEQ ID NO: 39, 79-96, and 97.

15. The therapeutic compound of claim 1, wherein the second protein is PD-L1 isoform C (SEQ ID NO:22) and wherein the API is a microRNA, the microRNA consisting of a nucleic acid sequence, the nucleic acid sequence being at least 95% identical to a microRNA sequence selected from the group consisting of SEQ ID NO: 39, 79-96, and 97.

16. The therapeutic compound of claim 1, wherein the second protein is PD-L1 isoform C (SEQ ID NO:22) and wherein the API is a microRNA consisting of a nucleic acid sequence selected from the group consisting of SEQ ID NO: 39, 79-96, and 97.

17. The therapeutic compound of claim 1, wherein the second protein is PD-L1 isoform A (SEQ ID NO:21) and wherein the API is an antisense oligonucleotide, the antisense oligonucleotide being 15-25 nucleotides in length and complementary to at least 15 contiguous nucleotides of SEQ ID NO:4.

18. The therapeutic compound of claim 1, wherein the second protein is PD-L1 isoform C (SEQ ID NO:22) and wherein the API is an antisense oligonucleotide, the antisense oligonucleotide being 15-25 nucleotides in length and complementary to at least 15 contiguous nucleotides of SEQ ID NO:5.

19. The therapeutic compound of claim 1, wherein the first protein is a PD-L1 binding peptide consisting of an amino acid sequence, the amino acid sequence being at least 90% identical to a peptide sequence selected from the group consisting of SEQ ID NO:60-65.

20. The therapeutic compound a of claim 1, wherein the first protein is a PD-L1 binding peptide consisting of an amino acid sequence selected from the group consisting of SEQ ID NO:60-65.
